(19) United States
(12) Patent Application Publication
(10) Pub. No.: US 2011/0130347 A1
(43) Pub. Date: Jun. 2, 2011

(54) IDENTIFICATION OF NEW SPLICE-VARIANTS OF G-PROTEIN COUPLED RECEPTOR EP3 AND USES THEREOF

(75) Inventors: Stefan Golz, Mulheim an der Ruhr (DE); Andreas Geerts, Wuppertal (DE)

(73) Assignee: Bayer Schering Pharma Aktiengesellschaft, Berlin (DE)

(21) Appl. No.: 12/602,767

(22) PCT Filed: May 20, 2008
(86) PCT No.: PCT/EP08/04007

§ 371 Date: Jun. 1, 2010

(30) Foreign Application Priority Data
Jun. 2, 2007 (EP) 07010926.9

(51) Int. Cl. A61K 38/17; C12Q 1/68; C12N 5/10; C12N 15/63; G01N 33/68; C07K 14/47; C07H 21/04; G01N 33/53

(52) U.S. Cl. 514/21.2; 435/6.17; 435/325; 435/320.1; 436/501; 436/86; 530/350; 536/23.5

(57) ABSTRACT

The present invention is directed to a polynucleotide sequence of the novel G-Protein Coupled Receptors EP3-11 OR EP3-12. The present invention provides polynucleotide sequences comprising the nucleic acid sequence SEQ ID NO: 20 or SEQ ID NO: 21 or nucleic acid sequences that hybridize to SEQ ID NO: 20 or SEQ ID NO: 21 or its complimentary strand having at least 40% sequence identity. The invention also provides the human EP3-11 or EP3-12 as targets for the identification of compounds useful for the treatment and prevention of cardiovascular diseases, inflammation, reproduction disorders and cancer as a result of relative quantification of the mRNA distribution in different human tissues by expression profiling. The invention also provides assays for the identification of compounds modulating EP3-11 or EP3-12.

### TECHNICAL FIELD OF THE INVENTION

The present invention is in the field of molecular biology; more particularly, the present invention describes the nucleic acid sequences and an amino acid sequences of two novel human EP3 splice variants (EP3-11 and EP3-12) and its regulation for therapeutic and diagnostic purposes. Two new and expressed splice variants of EP3 were identified and diseases associated

### BACKGROUND OF THE INVENTION

### G-Protein Coupled Receptors

The EP3 receptor is a seven transmembrane G protein coupled receptor (GPCR). Many medically significant biological processes are mediated by signal transduction pathways that involve G-proteins. [Lefkowitz et al. 1991]. The family of G-protein coupled receptors (GPCR) includes receptors for hormones, neurotransmitters, growth factors, and viruses. Specific examples of GPCRs include receptors for such diverse agents as dopamine, calcitonine, adrenergic hormones, endotheline, cAMP, adenosine, acetylcholine, serotonine, histamine, thrombin, kinine, follicle stimulating hormone, opsins, endothelial differentiation gene-1, rhodopsins, odorants, cytome-galovirus, G-proteins themselves, effector proteins such as phospholipase C, adenyl cyclase, and phosphodiesterase, and actuator proteins such as protein kinase A and protein kinase C.

GPCRs possess seven conserved membrane-spanning domains connecting at least eight divergent hydrophilic loops. GPCRs, also known as seven trans-membrane, 7™, receptors, have been characterized as including these seven conserved hydrophobic stretches of about 20 to 30 amino acids, connecting at least eight divergent hydrophilic loops. Most GPCRs have single conserved cysteine residues in each of the first two extracellular loops, which form disulfide bonds that are believed to stabilize functional protein structure. The seven transmembrane regions are designated as TM1, TM2, TM3, TM4, TM5, TM6, and TM7. TM3 is being implicated with signal transduction. Phosphorylation and lipidation (palmitylation or farnesylation) of cysteine residues can influence signal transduction of some GPCRs. Most GPCRs contain potential phosphorylation sites within the third cytoplasmic loop and/or the carboxy terminus. For several GPCRs, such as the beta-adrenergic receptor, phosphorylation by protein kinase A and/or specific receptor kinases mediates receptor desensitization.

For some receptors, the ligand binding sites of GPCRs are believed to comprise hydrophilic sockets formed by several GPCR transmembrane domains. The hydrophilic sockets are surrounded by hydrophobic residues of the GPCRs. The hydrophilic side of each GPCR transmembrane helix is postulated to face inward and form a polar ligand binding site. TM3 is being implicated with several GPCRs as having a ligand binding site, such as the TM3 aspartate residue. TM5 serines, a TM6 asparagine, and TM6 or TM7 phenylalanines or tyrosines also are implicated in ligand binding.

GPCRs are coupled inside the cell by heterotrimeric G-proteins to various intracellular enzymes, ion channels, and transporters. Different G-protein alpha-subunits preferentially stimulate particular effectors to modulate various biological functions in a cell. Phosphorylation of cytoplasmic residues of GPCRs is an important mechanism for the regulation of some GPCRs. For example, in one form of signal transduction, the effect of hormone binding is the activation of the enzyme, adenylate cyclase, inside the cell. Enzyme activation by hormones is dependent on the presence of the nucleotide GTP. GTP also influences hormone binding. A G-protein connects the hormone receptor to adenylate cyclase. G-protein exchanges GTP for bound GDP when activated by a hormone receptor. The GTP-carrying form then binds to activated adenylate cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

Over the past 15 years, nearly 350 therapeutic agents targeting 7™ receptors have been successfully introduced into the market. This indicates that these receptors have an established, proven history as therapeutic targets. Clearly, there is a need for identification and characterization of further receptors which can play a role in preventing, ameliorating, or correcting dysfunctions or diseases including, but not limited to, infections such as bacterial, fungal, protozoan, and viral infections, particularly those caused by HIV viruses, cancers, allergies including asthma, cardiovascular diseases including acute heart failure, hypotension, hypertension, angina pectoris, myocardial infarction, hematological diseases, genito-urinary diseases including urinary incontinence and benign prostate hyperplasia, osteoporosis, and peripheral and central nervous system disorders including pain, Alzheimer's disease and Parkinson's disease.

### Taqman-Technology/Human Tissue Localisation

TaqMan is a recently developed technique, in which the release of a fluorescent reporter dye from a hybridisation probe in real-time during a polymerase chain reaction (PCR) is proportional to the accumulation of the PCR product. Quantification is based on the early, linear part of the reaction, and by determining the threshold cycle (CT), at which fluorescence above background is first detected.

Gene expression technologies may be useful in several areas of drug discovery and development, such as target identification, lead optimization, and identification of mechanisms of action. The TaqMan technology can be used to compare differences between expression profiles of normal tissue and diseased tissue. Expression profiling has been used in identifying genes, which are up- or downregulated in a variety of diseases. An interesting application of expression profiling is temporal monitoring of changes in gene expression during disease progression and drug treatment or in patients versus healthy individuals. The premise in this approach is that changes in pattern of gene expression in response to physiological or environmental stimuli (e.g. drugs) may serve as indirect clues about disease-causing genes or drug targets. Moreover, the effects of drugs with established efficacy on global gene expression patterns may provide a guidepost, or a genetic signature, against which a new drug candidate can be compared.

### EP3 Receptor

### Synonyms: PROSTAGLANDIN E RECEPTOR 3, EP3 SUBTYPE; PTGER3

By screening both a human kidney cDNA library with mouse Ptger3 cDNAs and a human uterus cDNA library with a degenerate oligonucleotide based on a conserved region of prostanoid receptors, Adam et al. [Adam et al. 1994] cloned cDNAs encoding 3 isoforms of PTGER3. The predicted 365-, 388-, and 390-amino acid proteins are identical through the first 359 amino acids, which include the 7 transmembrane domains. Adam et al. [Adam et al. 1994] stated that mouse and bovine also have multiple PTGER3 isoforms that differ primarily in their C-terminal regions. The human PTGER3 proteins share approximately 85% amino acid identity with mouse, rat, and bovine PTGER3 proteins. Binding assays performed on human PTGER3 proteins expressed in mammalian cells showed that the 3 isoforms have comparable ligand-binding properties.

Using transfection experiments, Kotani et al. [Kotani et al. 1995] demonstrated that the different PTGER3 isoforms have divergent downstream signaling pathways.

Prostaglandin E2 (PGE2) induces uterine contraction by increasing intracellular calcium. To investigate other functions of PGE2 in human uterus, Kotani et al. [Kotani et al. 2000] isolated 2 prostaglandin E receptor EP3 isoforms by RT-PCR using human uterus poly (A)+ RNA. These EP3 isoforms, named EP3-V and EP3-VI, are composed of 402 and 393 amino acid residues, respectively, which are unique compared with EP3 isoforms of other species. Their N-terminal 359 amino acid residues are identical to those of previously reported human EP3 isoforms, whereas the respective C termini of the 2 isoforms contain a novel amino acid sequence. EP3-V and EP3-VI mRNAs were detected abundantly in human uterus, whereas weak but substantial bands were detected in the lung and kidney in RT-PCR specific for each mRNA. In situ hybridization revealed EP3-V and EP3-VI mRNAs in the human myometrium, but not in the endometrium. Over a decade of intensive research several EP3 splice variants have been reported showing the vast interest in EP3 receptor. In 2003 two new receptor splice variants, EP3-13 and EP3-14, were postulated based on genetic linkage analysis (WO2003064471). Nevertheless, expression analysis revealed that these two new splice variants are not expressed in human tissues and therefore not suitable as screening targets (see FIGS. 36 and 37, respectively).

Fever, a hallmark of disease, is elicited by exogenous pyrogens, i.e., cellular components such as lipopolysaccharide (LPS) of infectious organisms, as well as by noninfectious inflammatory insults. Both stimulate the production of cytokines, such as interleukin-1-beta, that act on the brain as endogenous pyrogens. Fever can be suppressed by aspirin-like antiinflammatory drugs. As these drugs share the ability to inhibit prostaglandin biosynthesis, it appeared that a prostaglandin is important in fever generation. Whether prostaglandin E2 (PGE2) is a neural mediator of fever has been debated. PGE2 acts by interacting with 4 subtypes of PGE receptors: EP1, EP2, EP3, and EP4. Ushikubi et al. [Ushikubi et al. 1998] generated mice lacking each of these receptors by homologous recombination. Only mice lacking the EP3 receptor failed to show a febrile response to PGE2 and to either IL1B or LPS. The results established that PGE2 mediates fever generation in response to both endogenous and exogenous pyrogens by acting at the EP3 receptor. As described in the OMIM database (Online Mendelian Inheritance in Man; Johns Hopkins University).

### SUMMARY OF THE INVENTION

The invention relates to nucleotide sequences which encode two novel human EP3 splice variants (EP3-11 and EP3-12). In the following EP3-11 and EP3-12 designate polypeptides having the sequence of or being homologous to SEQ ID NO:22 or SEQ ID NO:23, and having EP3 activity. EP3-11 and EP3-12 further contemplates various polypeptides arising from post-translational modifications of the polypeptide including but not limited to acetylation, carboxylation, glycosylation, phosphorylation, lipidation and acylation. The invention relates to nucleic acid molecules encoding EP3-11 or EP3-12 and polypeptides having EP3-activity, and to their use in the diagnosis or treatment of diseases associated with expression of EP3-11 or EP3-12.

Although the prior art teaches away from the present invention it is shown that the newly identified EP3 splice variants EP3-11 and EP3-12 are selectively expressed in human tissue (see FIGS. 34 and 35, respectively), in contrast to other postulated splice variants whose expression was not detectable in the examined human tissues (see FIGS. 36 and 37, respectively).

The invention relates to nucleotide sequences which encode EP3 splice variants comprising sequences with adjacently spliced exon 7 and exon 12.

The invention relates to nucleotide sequences which encode EP3 splice variants comprising sequences with adjacently spliced exon 9 and exon 12.

It is an object of the invention to provide reagents and methods for regulating the expression and activity of human EP3-11 and EP3-12 for the treatment of cardiovascular diseases, inflammation, reproduction disorders and cancer. This and other objects of the invention are provided by one or more of the embodiments described below.

Another object of the invention is a method of screening for agents which can regulate the activity of EP3-11 or EP3-12. A test compound is contacted with a polypeptide comprising the amino acid sequence selected of the group consisting of SEQ ID NO:22 or SEQ ID NO:23 or a polypeptide which exhibits EP3-11 or EP3-12 activity and is encoded by a polynucleotide hybridizing under stringent conditions to polynucleotide shown in SEQ ID NO:20 or SEQ ID NO:21; and binding of the test compound to EP3-11 or EP3-12 is detected, wherein a test compound which binds to the polypeptide is identified as a potential therapeutic agent for decreasing the activity of EP3-11 or EP3-12. Another embodiment of the invention is a method of screening for agents which can regulate the activity of EP3-11 or EP3-12. A test compound contacted with a polypeptide comprising the amino acid sequence selected from a group consisting of SEQ ID NO:22 or SEQ ID NO:23 or a polypeptide which exhibits EP3-11 or EP3-12 activity and is encoded by a polynucleotide hybridizing under stringent conditions to polynucleotide shown in SEQ ID NO:20 or SEQ ID NO:21; and EP3-11 or EP3-12 activity of the polypeptide is detected, wherein a test compound which increases EP3-11 or EP3-12 activity is identified as a potential therapeutic agent for increasing the activity of EP3-11 or EP3-12, and wherein a test compound which decreases EP3-11 or EP3-12 activity of the polypeptide is identified as a potential therapeutic agent for decreasing the activity of EP3-11 or EP3-12.

Another object of the invention is a method of screening for agents which can regulate the activity of EP3-11 or EP3-12. A test compound is contacted with a polynucleotide comprising the sequence selected of the group consisting of SEQ ID NO:20 or SEQ ID NO:21 or a polynucleotide which encodes a polypeptide exhibiting EP3-11 or EP3-12 activity and hybridizes under stringent conditions to the polynucleotide shown in SEQ ID NO:20 or SEQ ID NO:21; and binding of the test compound to the polynucleotide is detected, wherein a test compound which binds to the polynucleotide is identified as a potential therapeutic agent for decreasing the activity of EP3-11 or EP3-12.

Another object of the invention is a method of screening for agents which can regulate the activity of EP3-11 or EP3-12. A test compound is contacted with a product encoded by a polynucleotide which comprises the nucleotide sequence shown in SEQ ID NO:20 or SEQ ID NO:21; and binding of the test compound to the product is detected, wherein a test compound which binds to the product is identified as a potential agent for regulating the activity of EP3-11 or EP3-12.

Another object of the invention is a method of reducing the activity of EP3-11 or EP3-12. A cell is contacted with a reagent which specifically binds to a polynucleotide encoding EP3-11 OR EP3-12 or the EP3-11 OR EP3-12 polypeptide. EP3-11 OR EP3-12 activity is thereby reduced.

Another object of the invention is a method of increasing the activity of EP3-11 OR EP3-12. A cell is contacted with a reagent which specifically binds to a polynucleotide encoding EP3-11 OR EP3-12 or the EP3-11 OR EP3-12 polypeptide. EP3-11 OR EP3-12 activity is thereby increased.

Another object of the invention is the antisense DNA of DNA encoding EP3-11 OR EP3-12; cloning or expression vectors containing nucleic acid encoding EP3-11 OR EP3-12; host cells or organisms transformed with expression vectors containing nucleic acid encoding EP3-11 OR EP3-12; a method for the production and recovery of purified EP3-11 OR EP3-12 from host cells: purified protein, EP3-11 OR EP3-12, which can be used to identify inhibitors or activators of signal transduction involving EP3-11 OR EP3-12; and methods of screening for ligands of EP3-11 OR EP3-12 using transformed cells.

### BRIEF DESCRIPTION OF THE DRAWINGS

FIG. 1 shows the nucleotide sequence of a EXON 1 polynucleotide (SEQ ID NO:1).

FIG. 2 shows the nucleotide sequence of a EXON 2 polynucleotide (SEQ ID NO:2).

FIG. 3 shows the nucleotide sequence of a EXON 3 polynucleotide (SEQ ID NO:3).

FIG. 4 shows the nucleotide sequence of a EXON 4 polynucleotide (SEQ ID NO:4).

FIG. 5 shows the nucleotide sequence of a EXON 5 polynucleotide (SEQ ID NO:5).

FIG. 6 shows the nucleotide sequence of a EXON 6 polynucleotide (SEQ ID NO:6).

FIG. 7 shows the nucleotide sequence of a EXON 7 polynucleotide (SEQ ID NO:7).

FIG. 8 shows the nucleotide sequence of a EXON 8 polynucleotide (SEQ ID NO:8).

FIG. 9 shows the nucleotide sequence of a EXON 9 polynucleotide (SEQ ID NO:9).

FIG. 10 shows the nucleotide sequence of a EXON 10 polynucleotide (SEQ ID NO:10).

FIG. 11 shows the nucleotide sequence of a EXON 11 polynucleotide (SEQ ID NO:11).

FIG. 12 shows the nucleotide sequence of a EXON 12 polynucleotide (SEQ ID NO:12).

FIG. 13 shows the nucleotide sequence of a EXON 2 long polynucleotide (SEQ ID NO:13).

FIG. 14 shows the nucleotide sequence of EXON 2×2 polynucleotide (SEQ ID NO:14)

FIG. 15 shows the nucleotide sequence of EXON 2×3 polynucleotide (SEQ ID NO:15)

FIG. 16 shows the nucleotide sequence of spliced Exon 7 to 12 (SEQ ID NO:16)

FIG. 17 shows the nucleotide sequence of spliced Exon 9 to 12 (SEQ ID NO:17)

FIG. 18 shows the nucleotide sequence of EST BG209275.1 (SEQ ID NO:18)

FIG. 19 shows the nucleotide sequence of EST BG192181.1 (SEQ ID NO:19)

FIG. 20 shows the nucleotide sequence of a EP3-11 polynucleotide (SEQ ID NO:20).

FIG. 21 shows the nucleotide sequence of a EP3-12 polynucleotide (SEQ ID NO:21).

FIG. 22 shows the amino acid sequence of a EP3-11 polypeptide (SEQ ID NO:22).

FIG. 23 shows the amino acid sequence of a EP3-12 polypeptide (SEQ ID NO:23).

FIG. 24 shows the relative expression of EP3-1 in various human tissues

FIG. 25 shows the relative expression of EP3-2 in various human tissues

FIG. 26 shows the relative expression of EP3-3 in various human tissues

FIG. 27 shows the relative expression of EP3-4 in various human tissues

FIG. 28 shows the relative expression of EP3-5 in various human tissues

FIG. 29 shows the relative expression of EP3-6 in various human tissues

FIG. 30 shows the relative expression of EP3-7 in various human tissues

FIG. 31 shows the relative expression of EP3-8 in various human tissues

FIG. 32 shows the relative expression of EP3-9 in various human tissues

FIG. 33 shows the relative expression of EP3-10 in various human tissues

FIG. 34 shows the relative expression of EP3-11 in various human tissues

FIG. 35 shows the relative expression of EP3-12 in various human tissues

FIG. 36 shows the relative expression of EP3-13 in various human tissues

FIG. 37 shows the relative expression of EP3-14 in various human tissues

FIG. 38 overview of EP3 splice variants

FIG. 39 shows nucleotide sequence of a primer useful for the invention—var1 (SEQ ID NO:24).

FIG. 40 shows nucleotide sequence of a primer useful for the invention—var1 (SEQ ID NO:25).

FIG. 41 shows nucleotide sequence of a probe useful for the invention—var1 (SEQ ID NO:26).

FIG. 42 shows nucleotide sequence of a primer useful for the invention—var2 (SEQ ID NO:27).

FIG. 43 shows nucleotide sequence of a primer useful for the invention—var2 (SEQ ID NO:28).

FIG. 44 shows nucleotide sequence of a probe useful for the invention—var2 (SEQ ID NO:29).

FIG. 45 shows nucleotide sequence of a primer useful for the invention—var3 (SEQ ID NO:30).

FIG. 46 shows nucleotide sequence of a primer useful for the invention—var3 (SEQ ID NO:31).

FIG. 47 shows nucleotide sequence of a probe useful for the invention—var3 (SEQ ID NO:32).

FIG. 48 shows nucleotide sequence of a primer useful for the invention—var4 (SEQ ID NO:33).

FIG. 49 shows nucleotide sequence of a primer useful for the invention—var4 (SEQ ID NO:34).

FIG. 50 shows nucleotide sequence of a probe useful for the invention—var4 (SEQ ID NO:35).

FIG. 51 shows nucleotide sequence of a primer useful for the invention—var5 (SEQ ID NO:36).

FIG. 52 shows nucleotide sequence of a primer useful for the invention—var5 (SEQ ID NO:37).

FIG. 53 shows nucleotide sequence of a probe useful for the invention—var5 (SEQ ID NO:38).

FIG. 54 shows nucleotide sequence of a primer useful for the invention—var6 (SEQ ID NO:39).

FIG. 55 shows nucleotide sequence of a primer useful for the invention—var6 (SEQ ID NO:40).

FIG. 56 shows nucleotide sequence of a probe useful for the invention—var6 (SEQ ID NO:41).

FIG. 57 shows nucleotide sequence of a primer useful for the invention—var7 (SEQ ID NO:42).

FIG. 58 shows nucleotide sequence of a primer useful for the invention—var7 (SEQ ID NO:43).

FIG. 59 shows nucleotide sequence of a probe useful for the invention—var7 (SEQ ID NO:44).

FIG. 60 shows nucleotide sequence of a primer useful for the invention—var8 (SEQ ID NO:45).

FIG. 61 shows nucleotide sequence of a primer useful for the invention—var8 (SEQ ID NO:46).

FIG. 62 shows nucleotide sequence of a probe useful for the invention—var8 (SEQ ID NO:47).

FIG. 63 shows nucleotide sequence of a primer useful for the invention—var9 (SEQ ID NO:48).

FIG. 64 shows nucleotide sequence of a primer useful for the invention—var9 (SEQ ID NO:49).

FIG. 65 shows nucleotide sequence of a probe useful for the invention—var9 (SEQ ID NO:50).

FIG. 66 shows nucleotide sequence of a primer useful for the invention—var10 (SEQ ID NO:51).

FIG. 67 shows nucleotide sequence of a primer useful for the invention—var10 (SEQ ID NO:52).

FIG. 68 shows nucleotide sequence of a probe useful for the invention—var10 (SEQ ID NO:53).

FIG. 69 shows nucleotide sequence of a primer useful for the invention—var11 (SEQ ID NO:54).

FIG. 70 shows nucleotide sequence of a primer useful for the invention—var11 (SEQ ID NO:55).

FIG. 71 shows nucleotide sequence of a probe useful for the invention—var11 (SEQ ID NO:56).

FIG. 72 shows nucleotide sequence of a primer useful for the invention—var12 (SEQ ID NO:57).

FIG. 73 shows nucleotide sequence of a primer useful for the invention—var12 (SEQ ID NO:58).

FIG. 74 shows nucleotide sequence of a probe useful for the invention—var12 (SEQ ID NO:59).

FIG. 75 shows nucleotide sequence of a primer useful for the invention—var13 (SEQ ID NO:60).

FIG. 76 shows nucleotide sequence of a primer useful for the invention—var13 (SEQ ID NO:61).

FIG. 77 shows nucleotide sequence of a probe useful for the invention—var13 (SEQ ID NO:62).

FIG. 78 shows nucleotide sequence of a primer useful for the invention—var14 (SEQ ID NO:63).

FIG. 79 shows nucleotide sequence of a primer useful for the invention—var14 (SEQ ID NO:64).

FIG. 80 shows nucleotide sequence of a probe useful for the invention—var14 (SEQ ID NO:65).

FIG. 81 shows the exon structure of the EP3 isoforms and variants. X-axis: exon (number), Y-axis: isoform/variant number

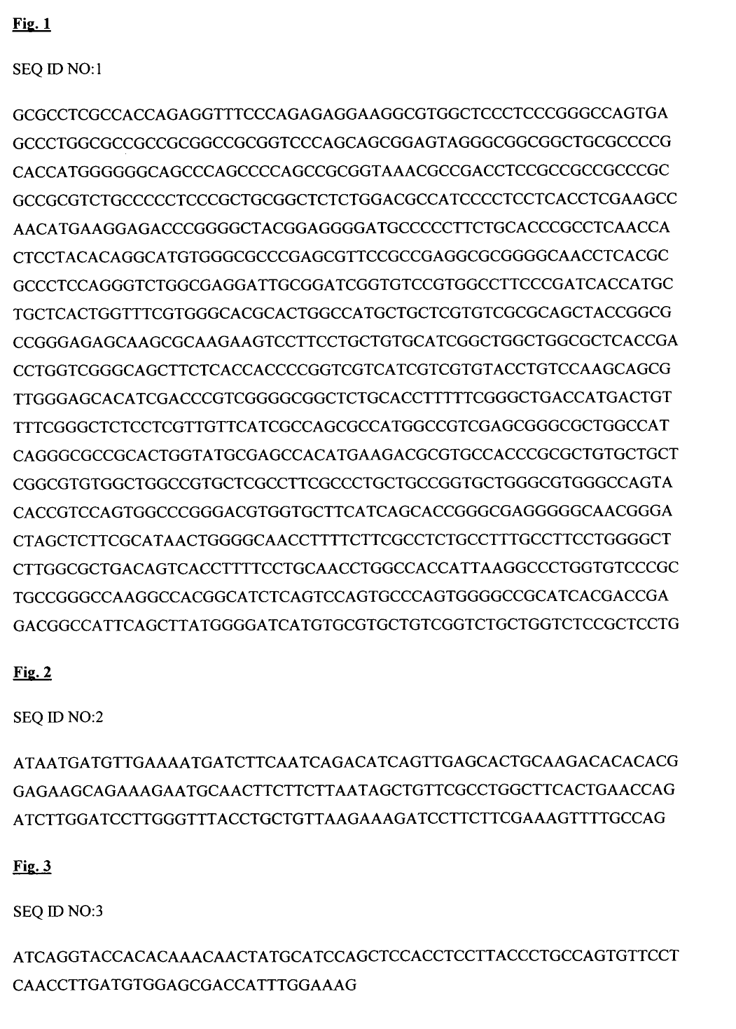

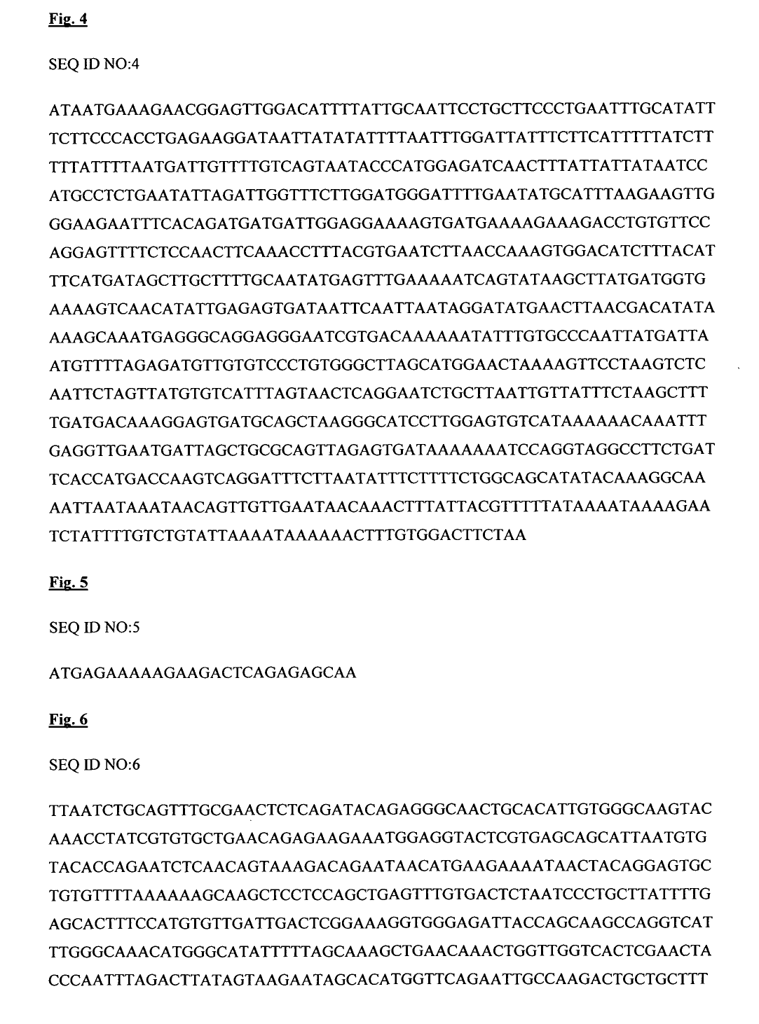

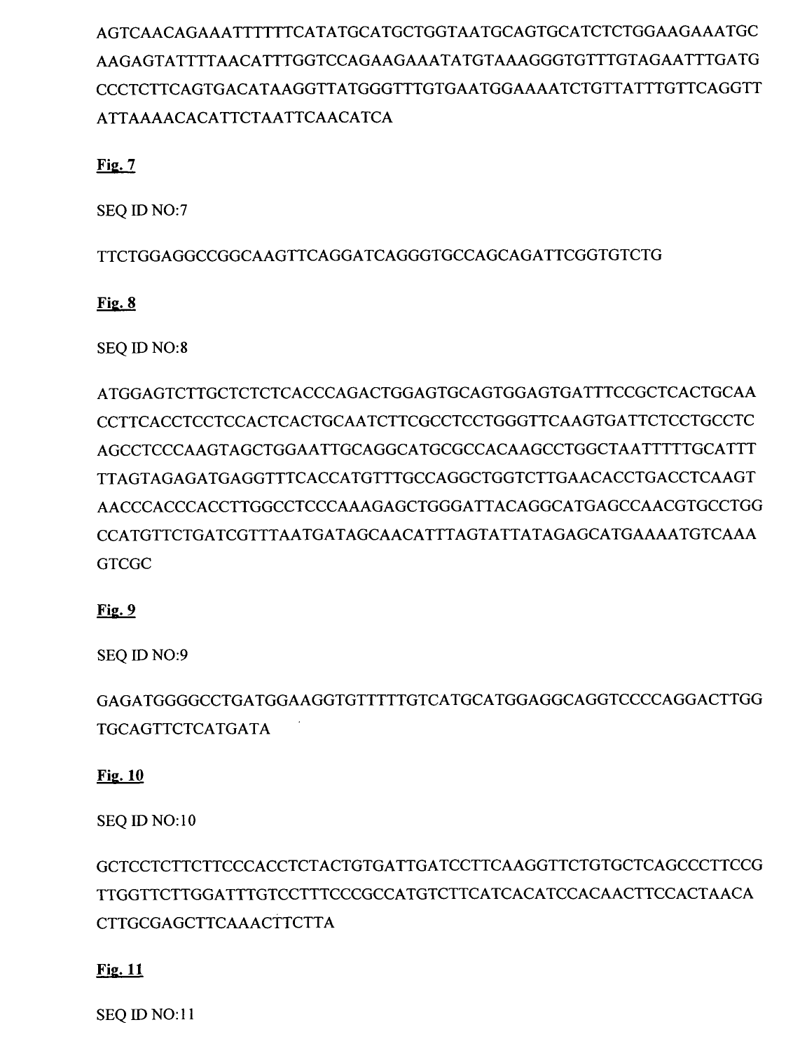

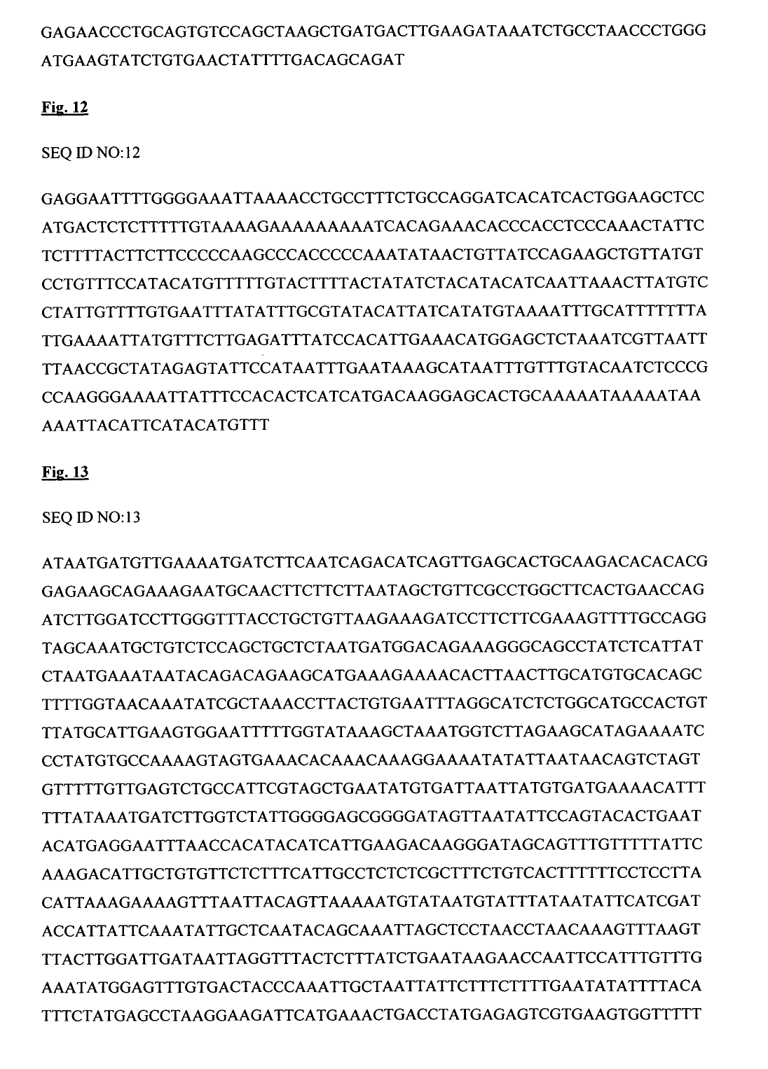

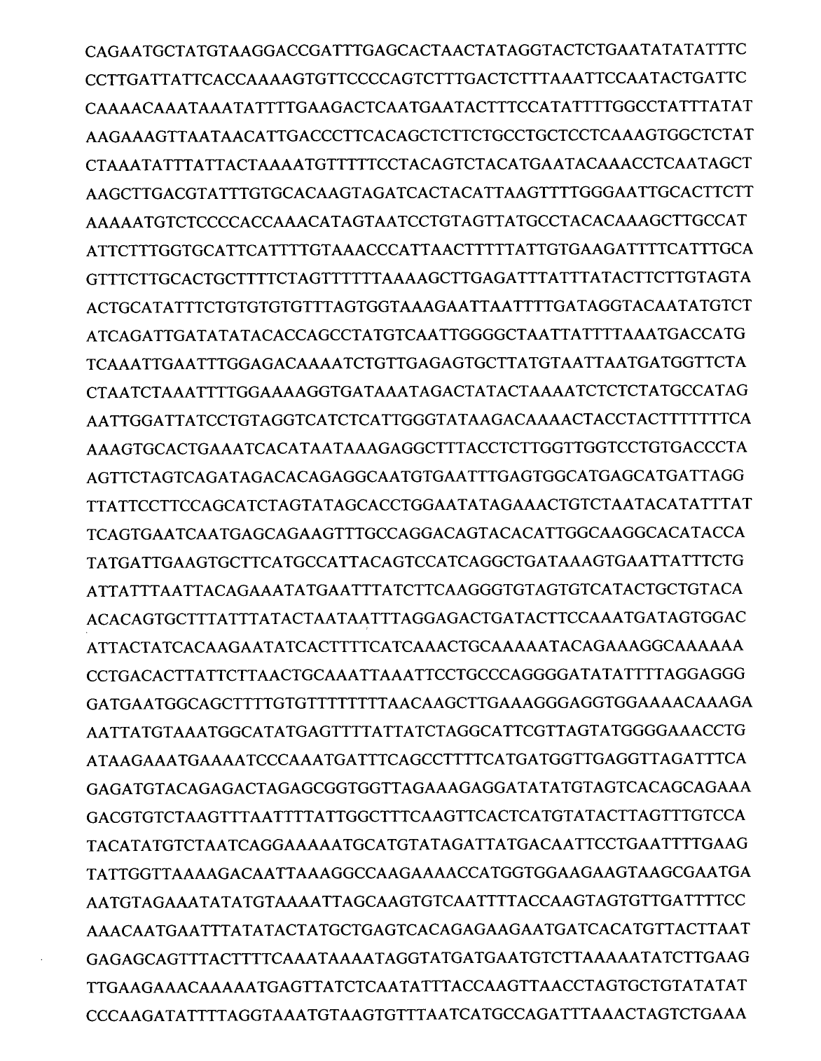

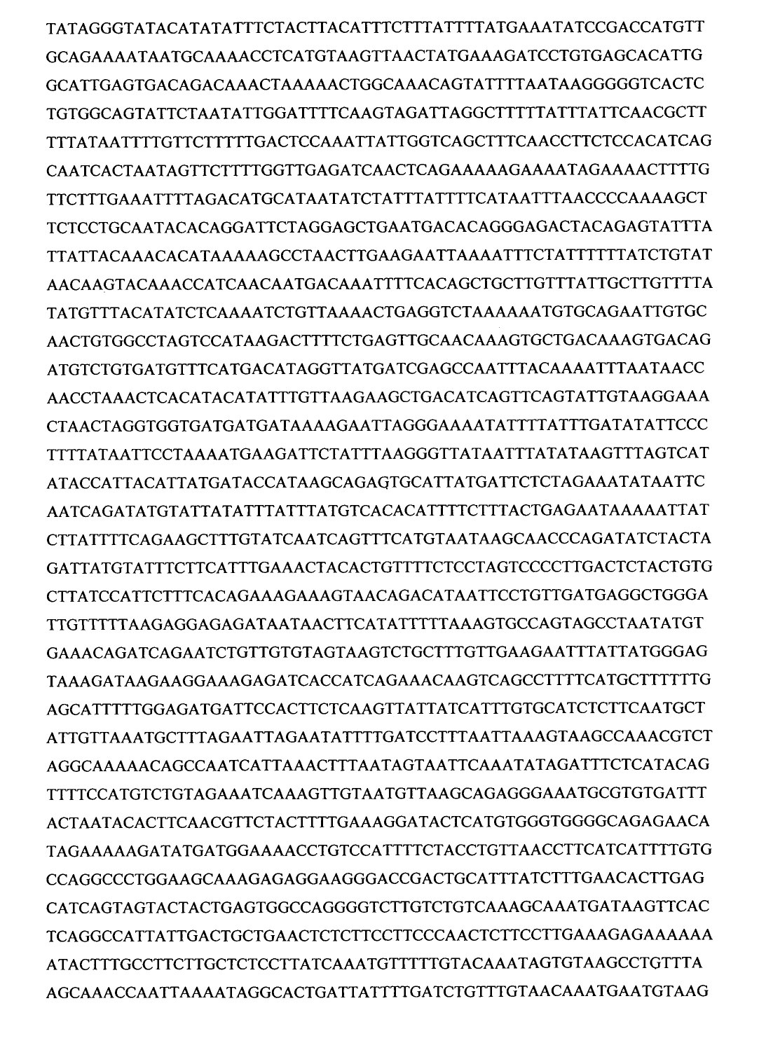

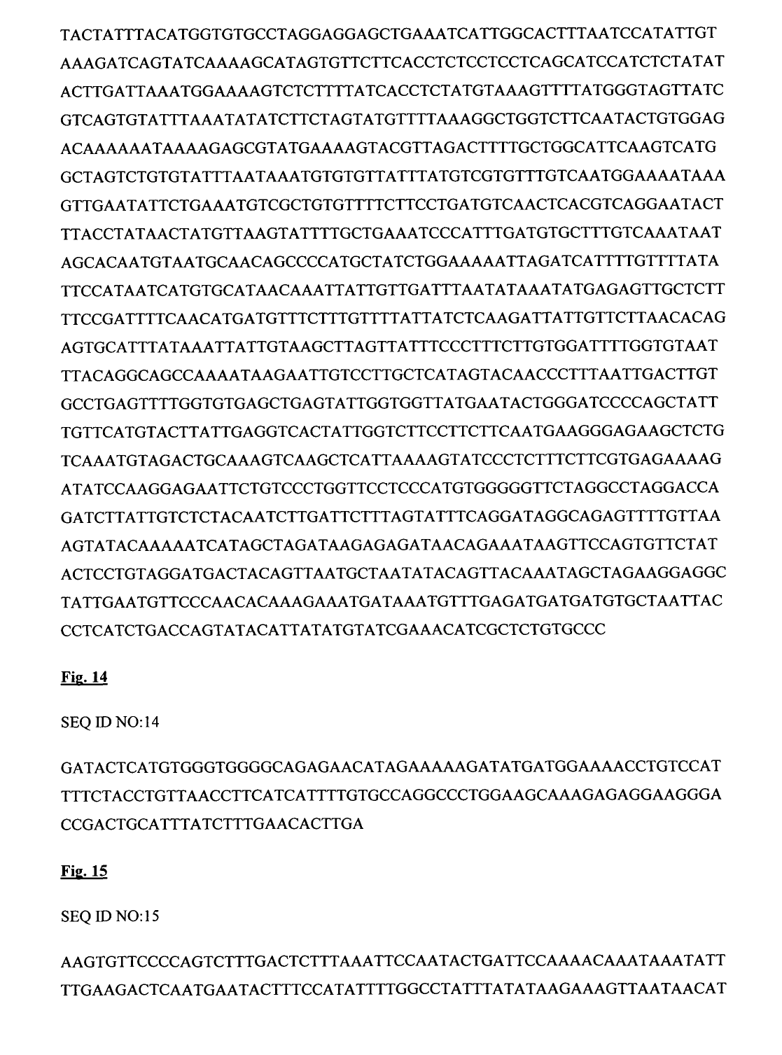

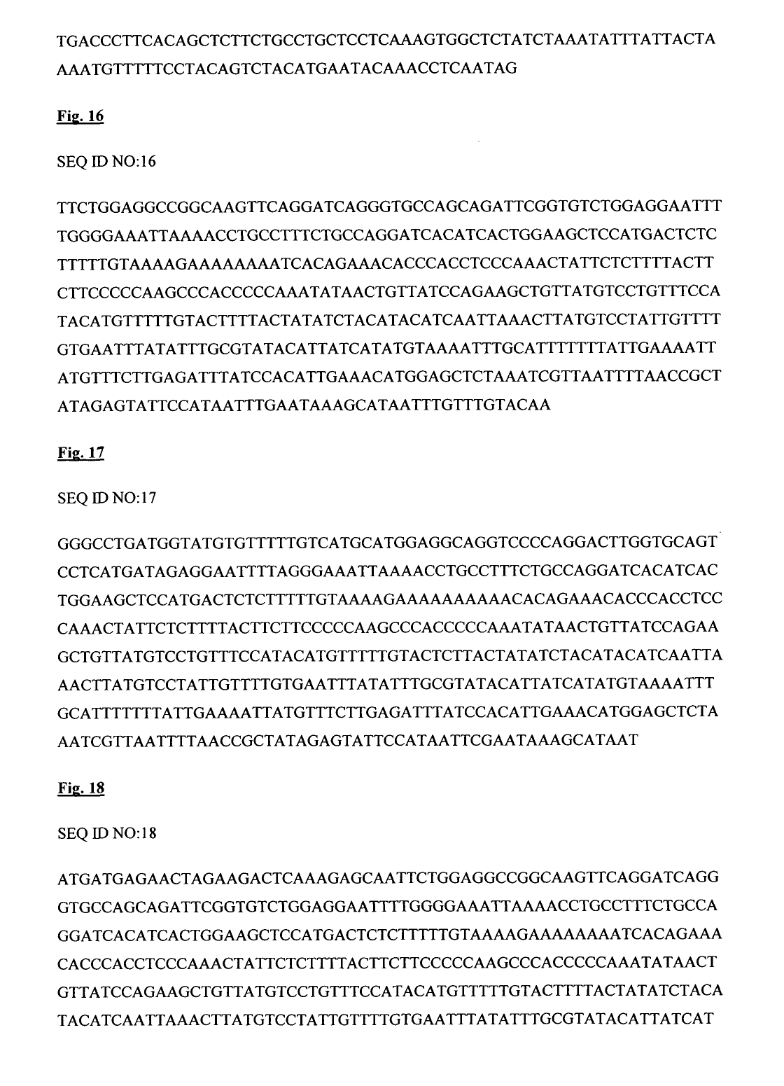

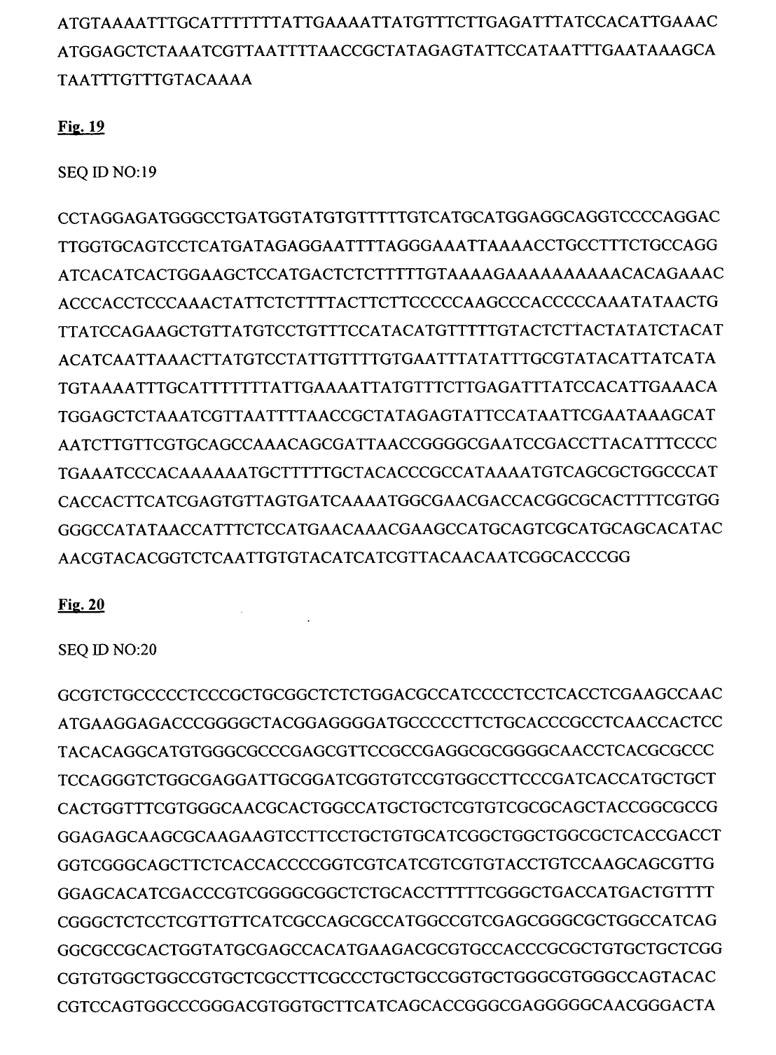

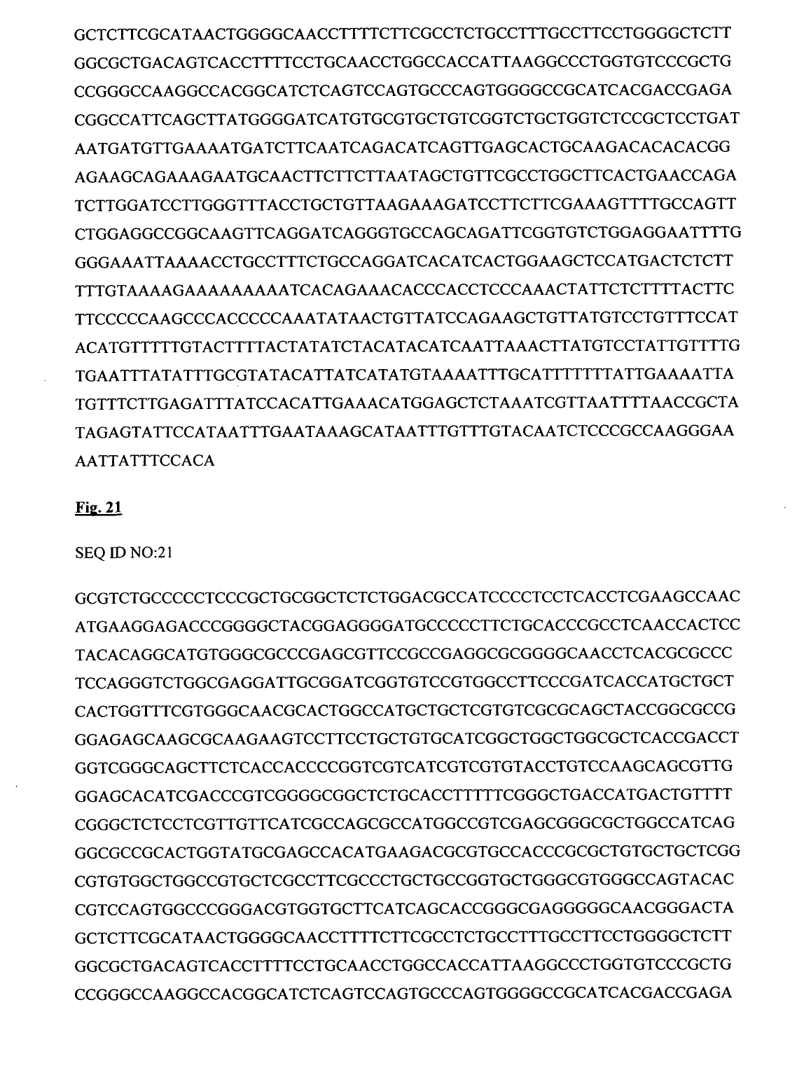

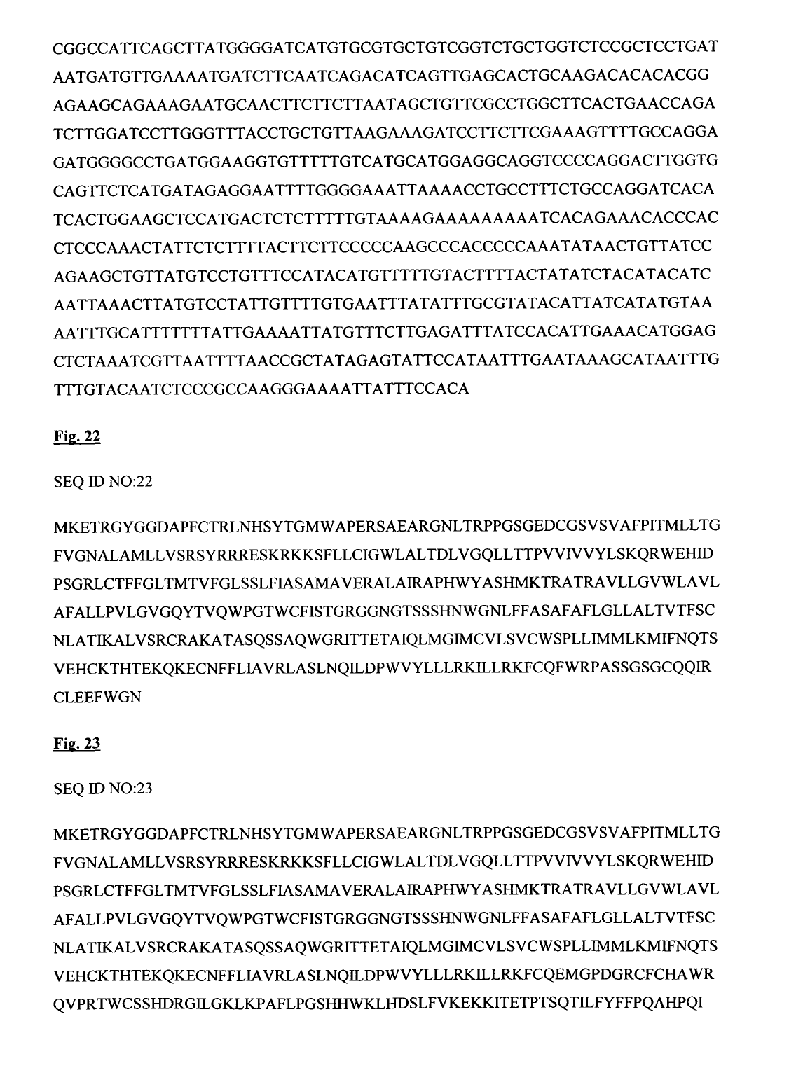

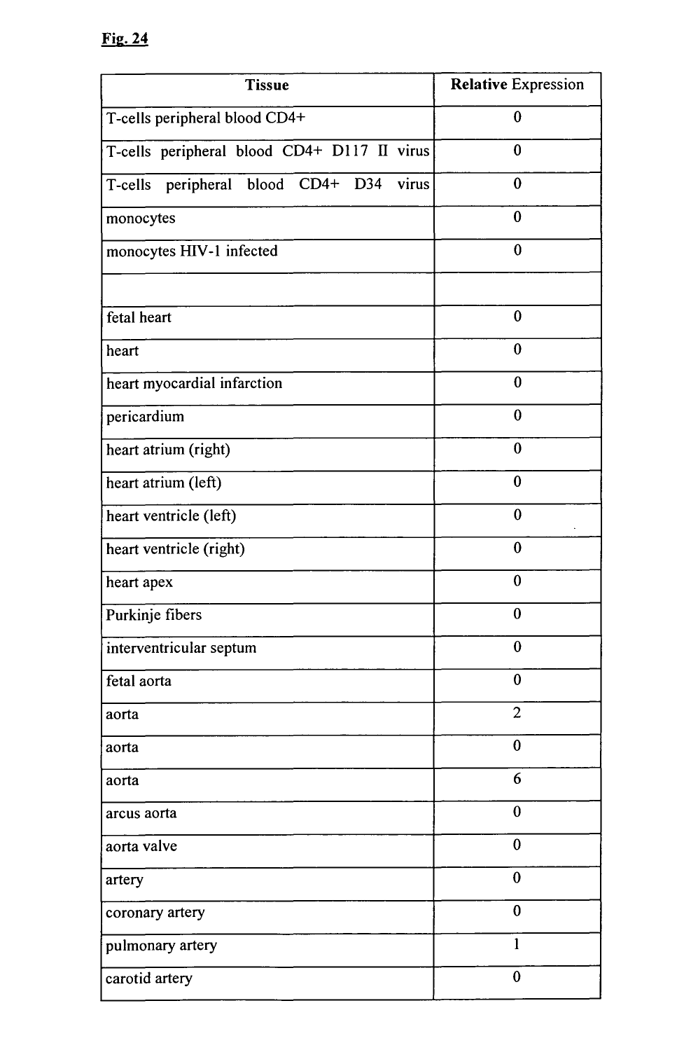

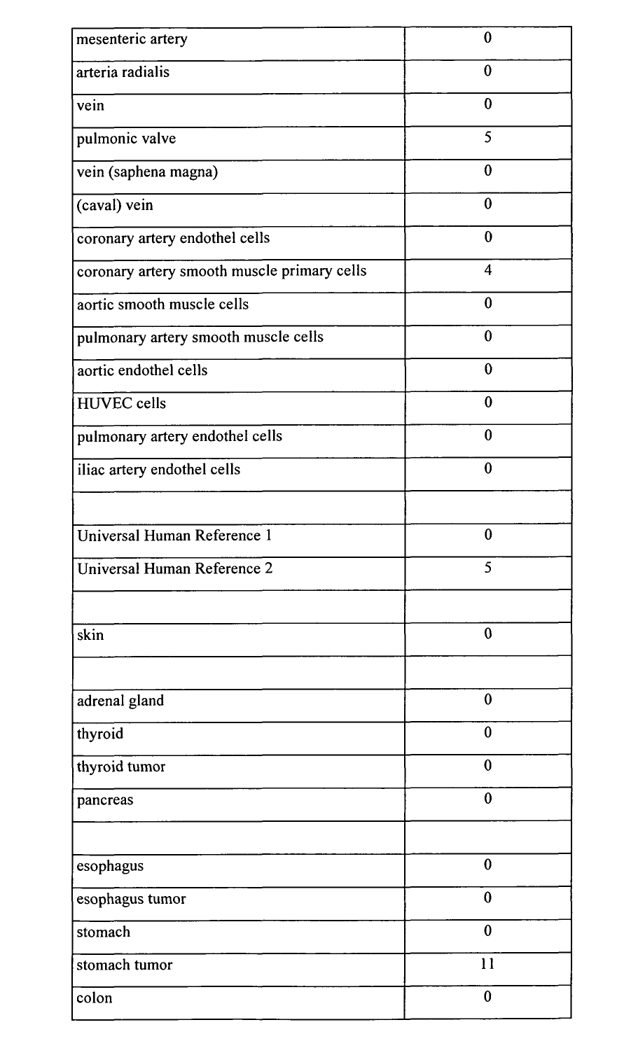

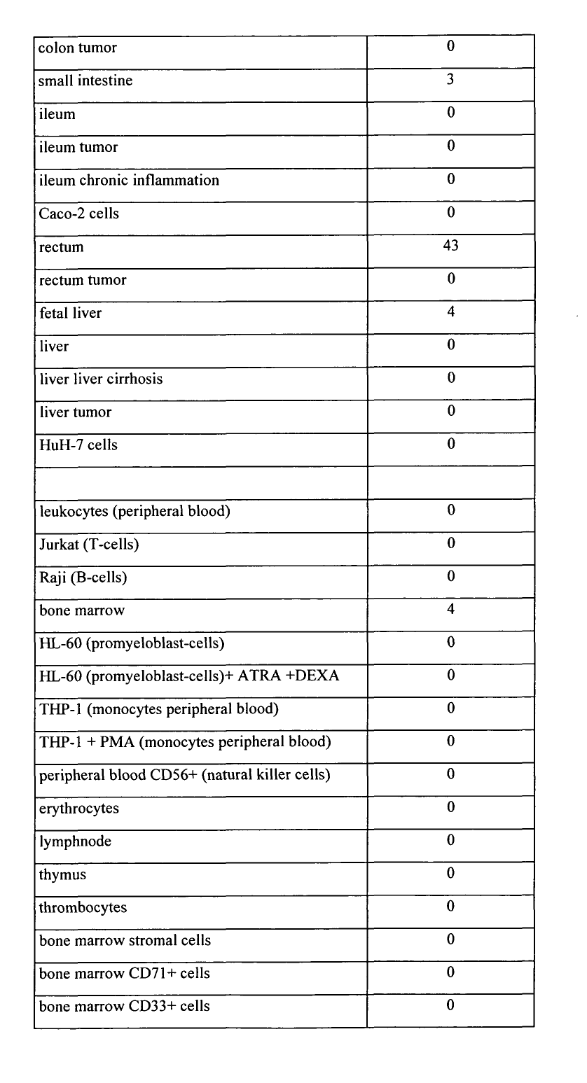

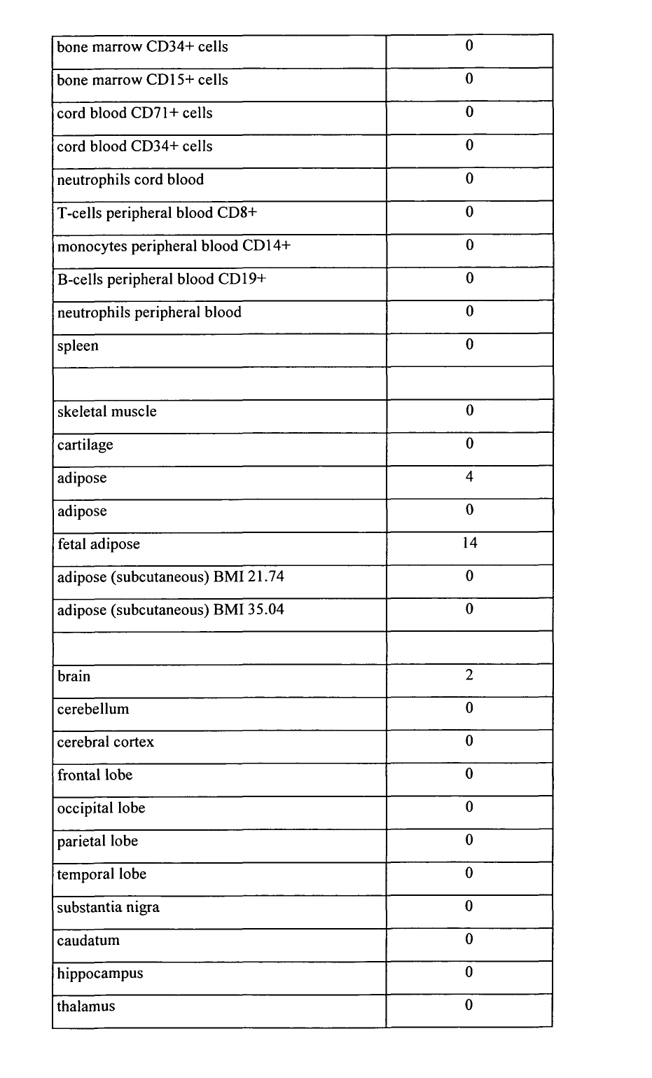

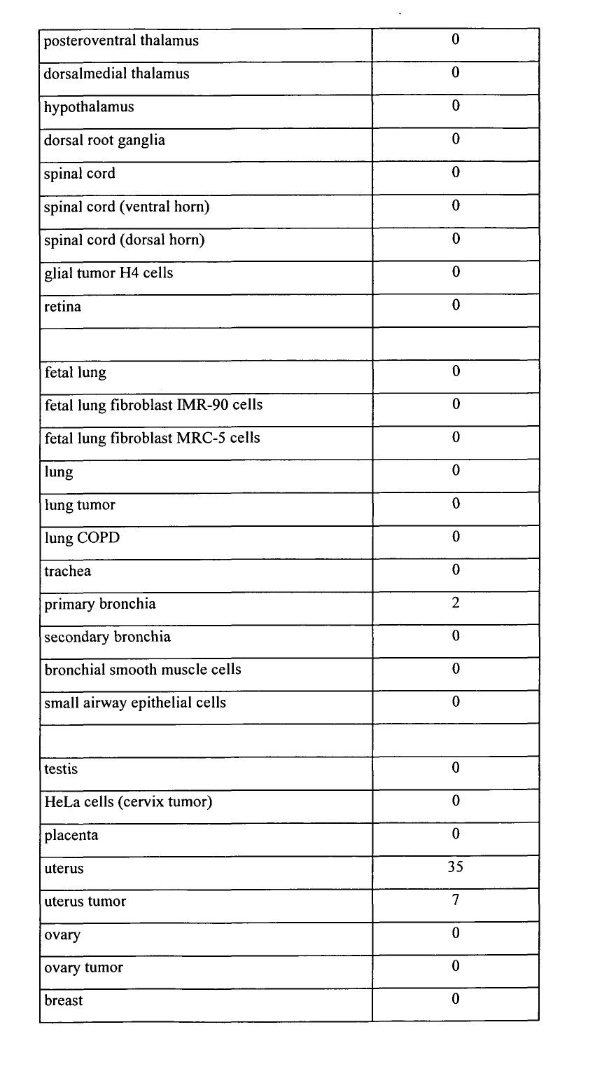

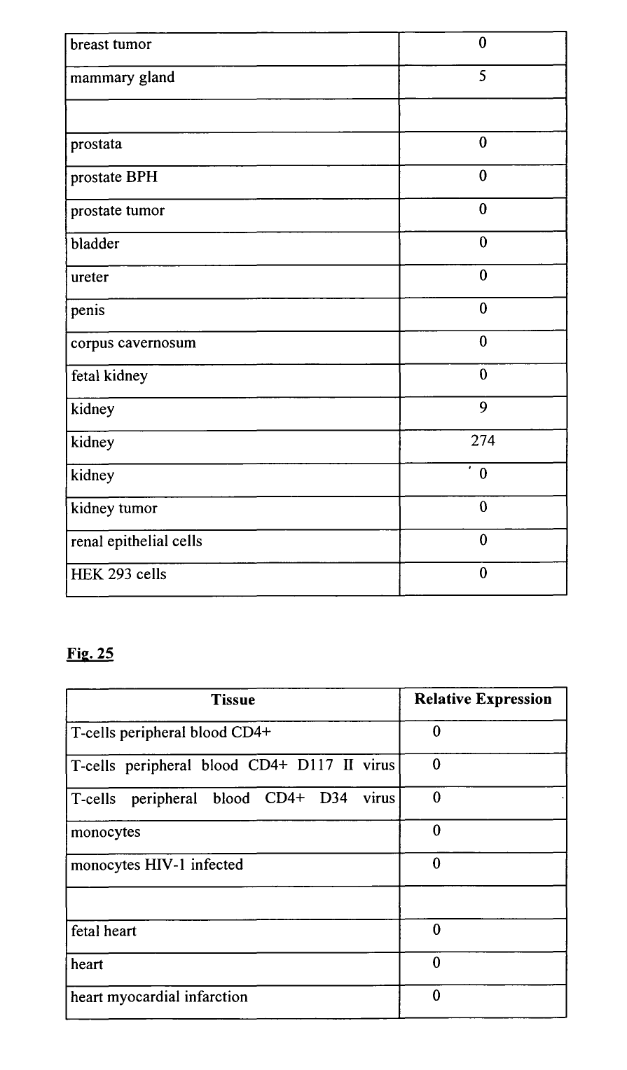

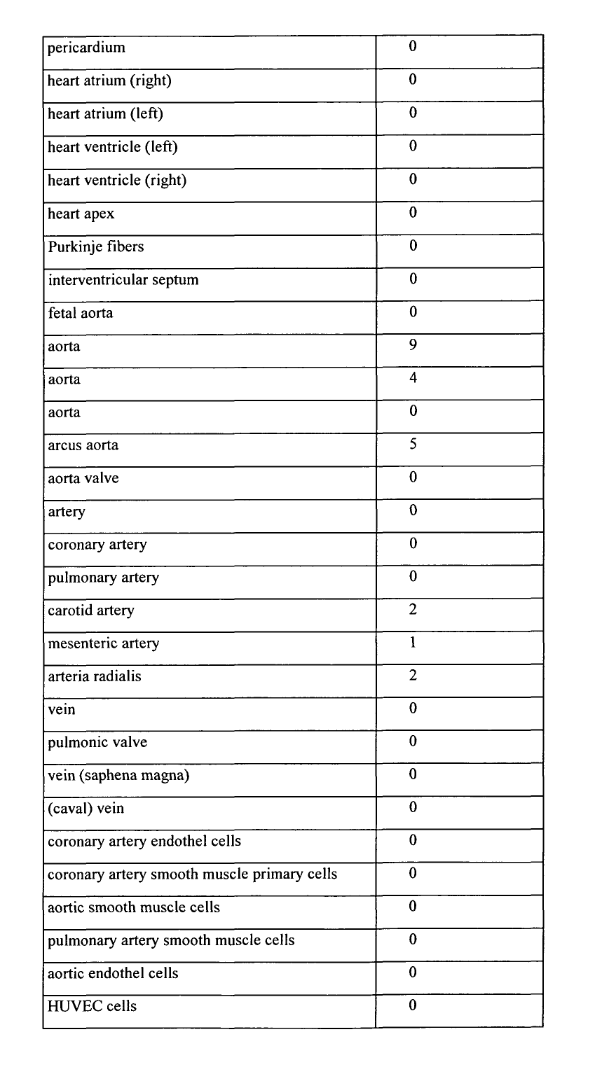

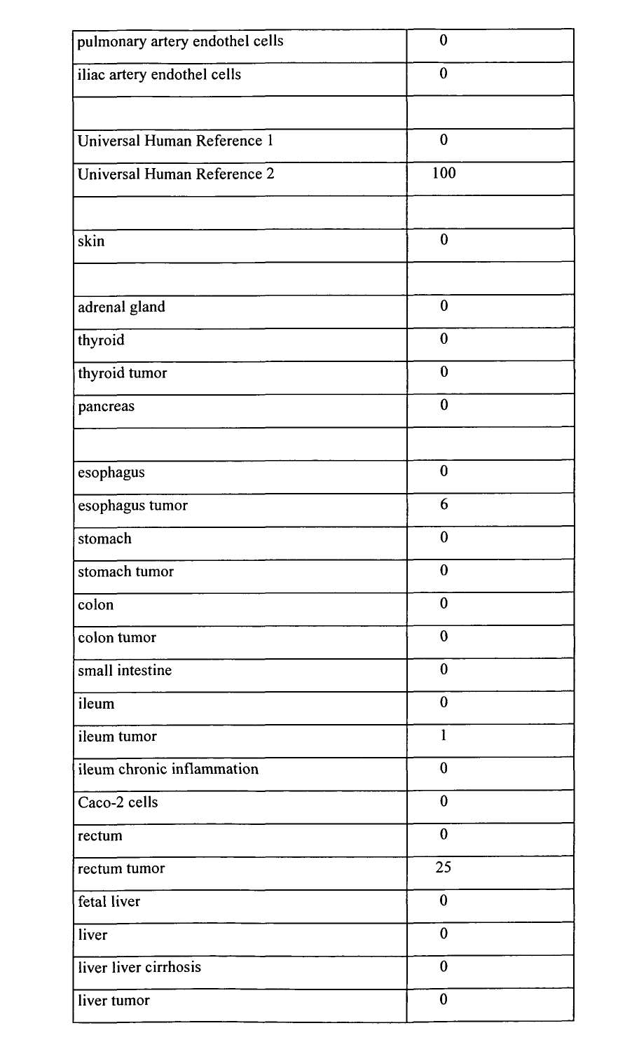

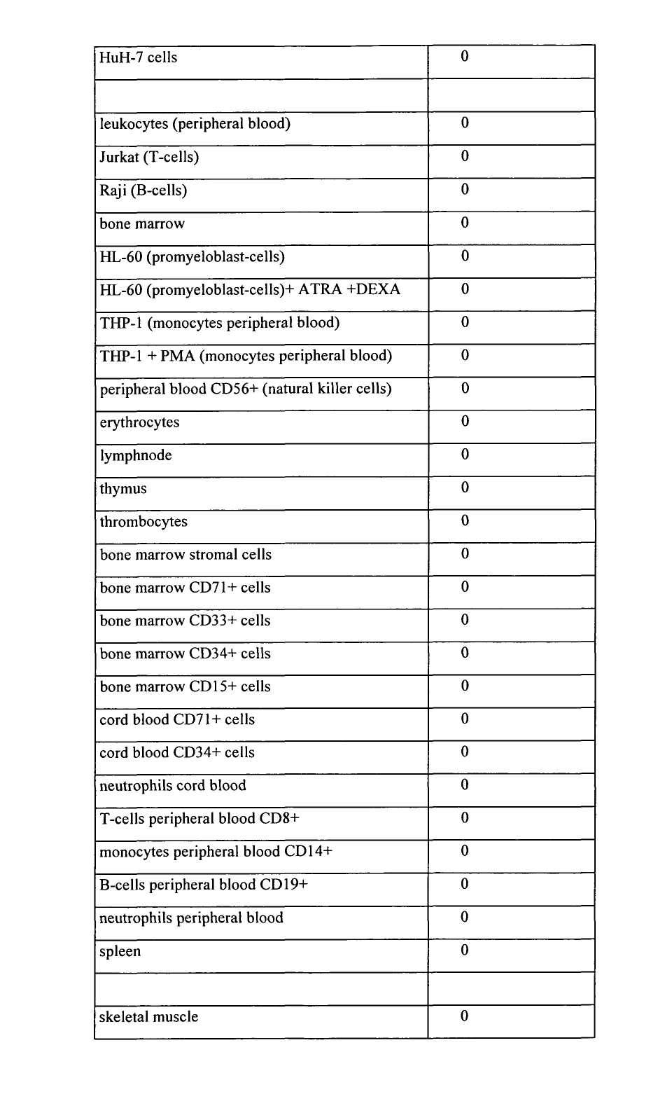

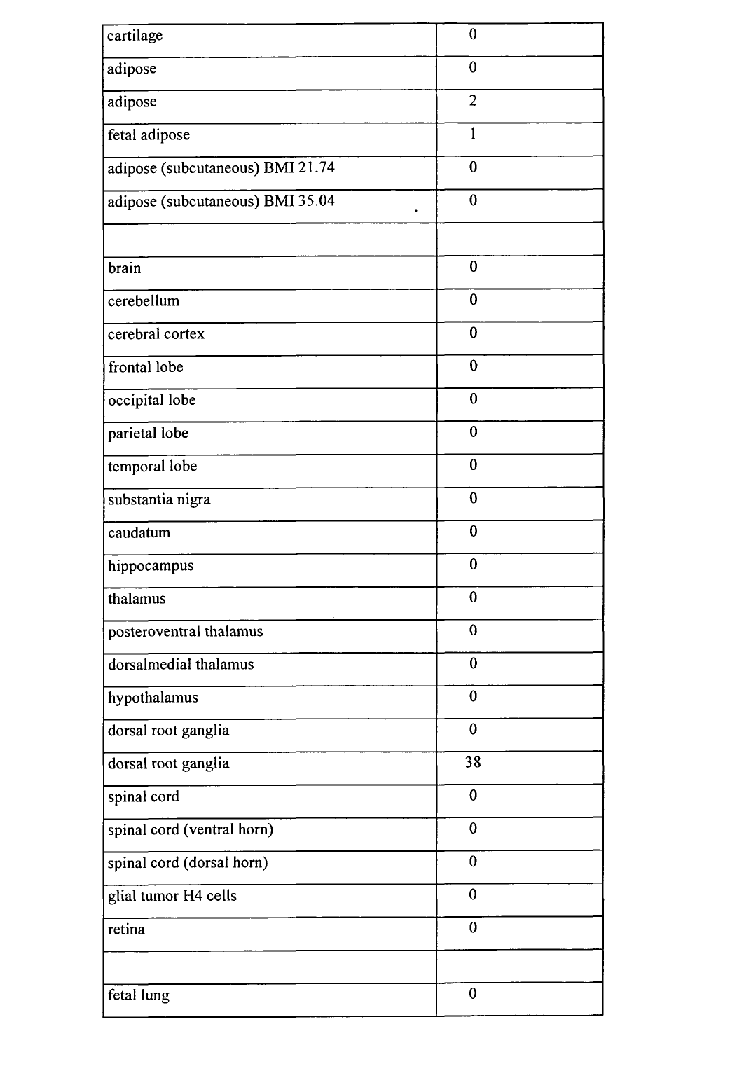

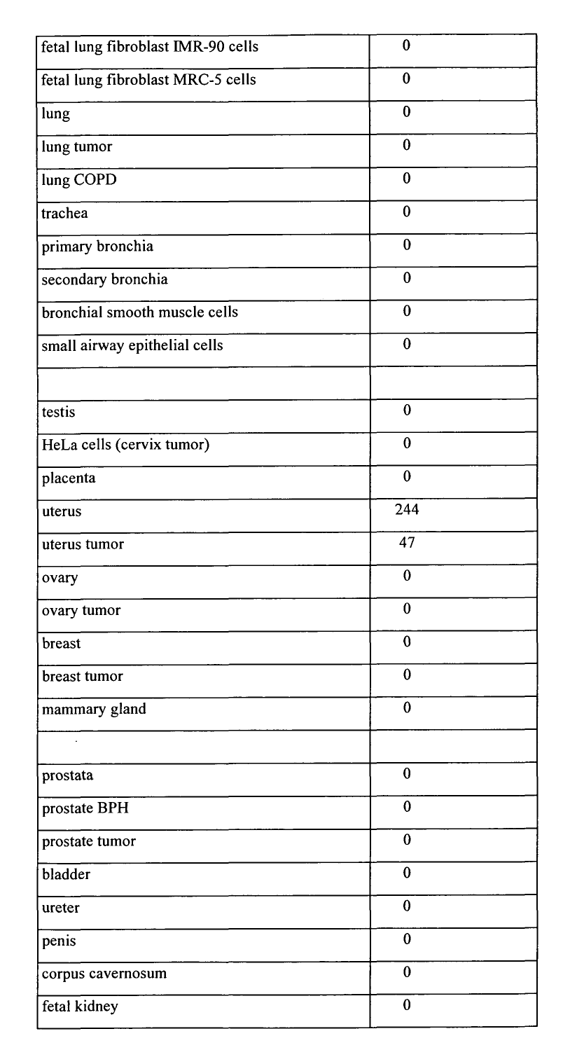

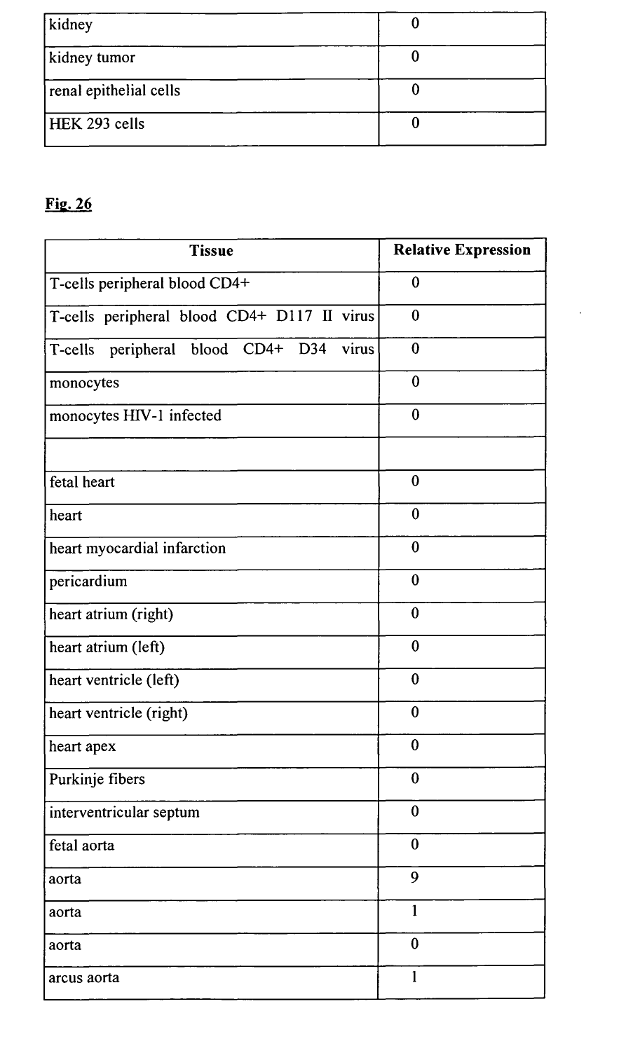

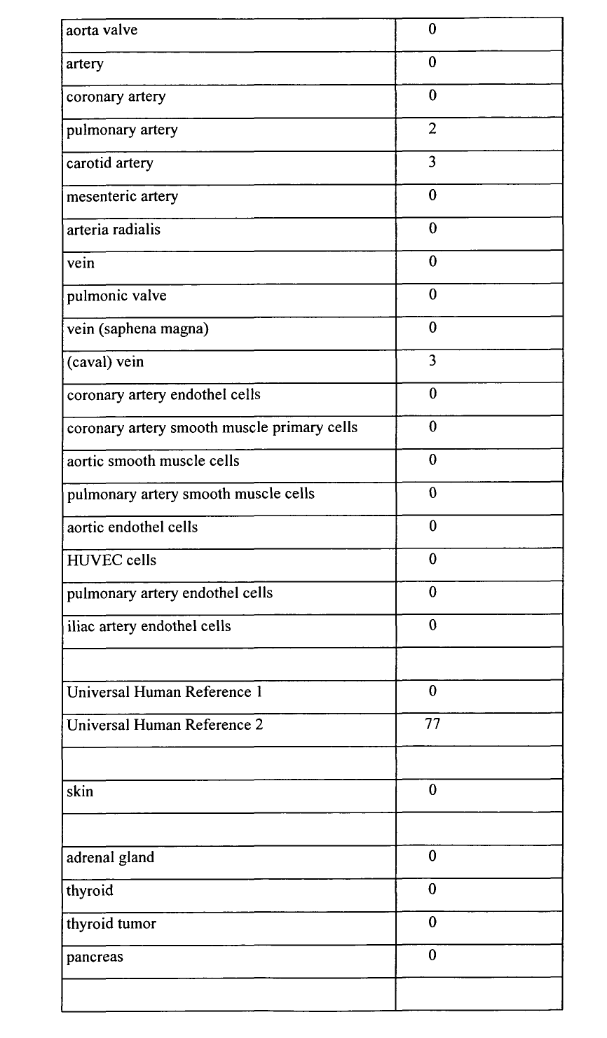

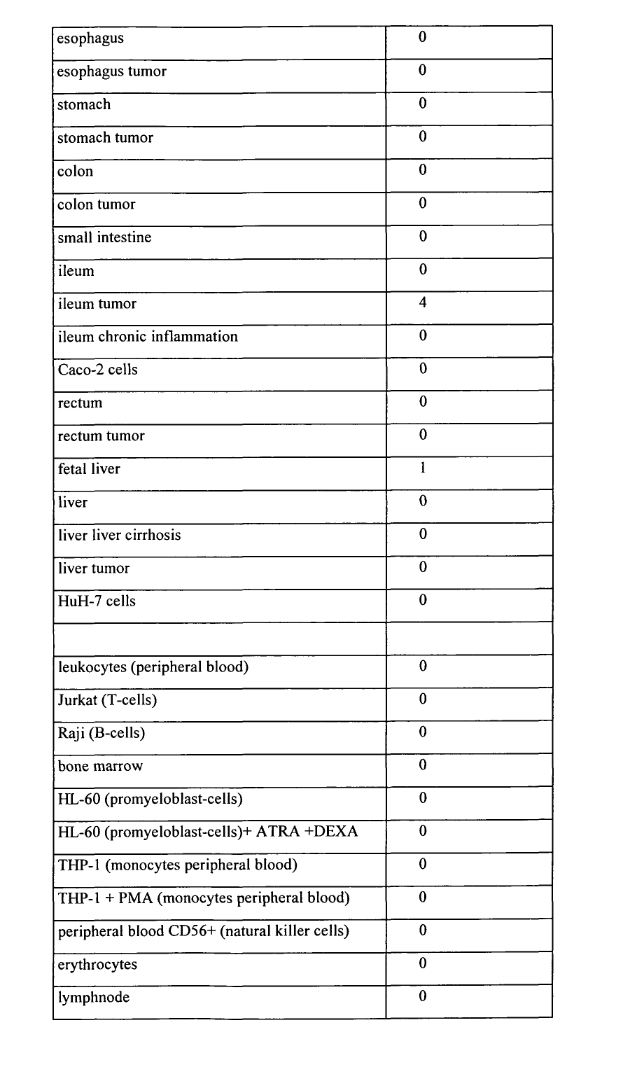

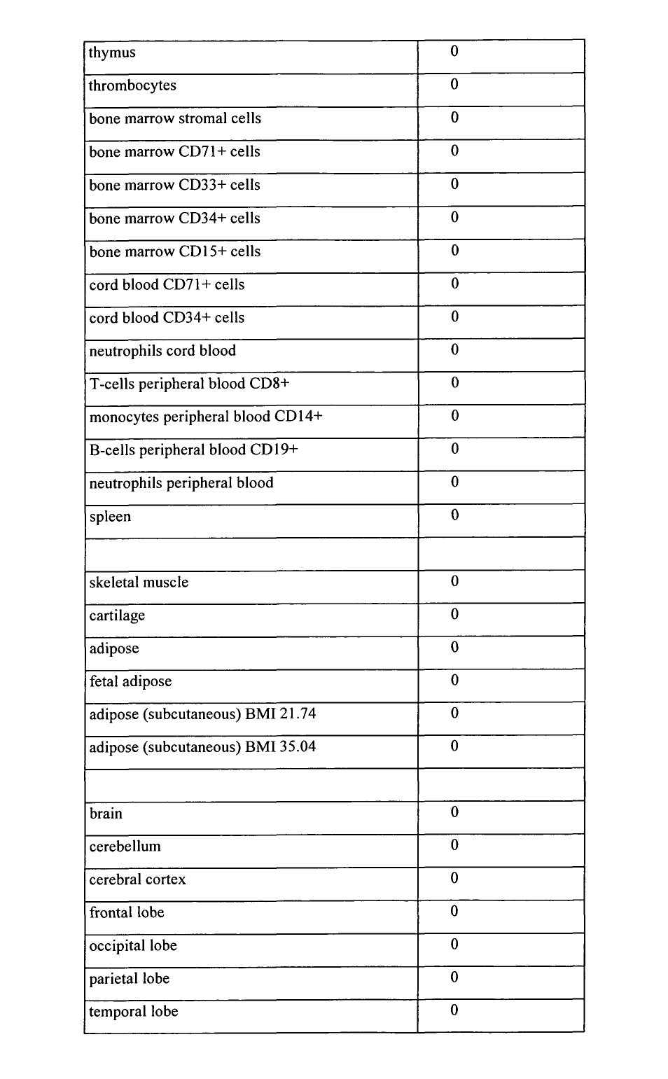

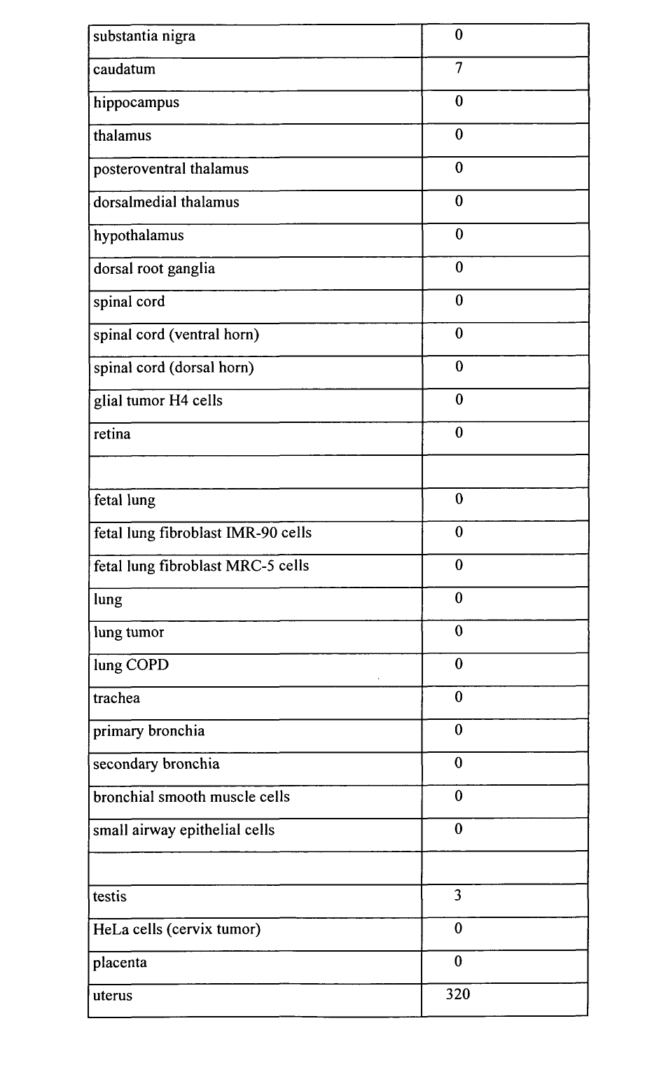

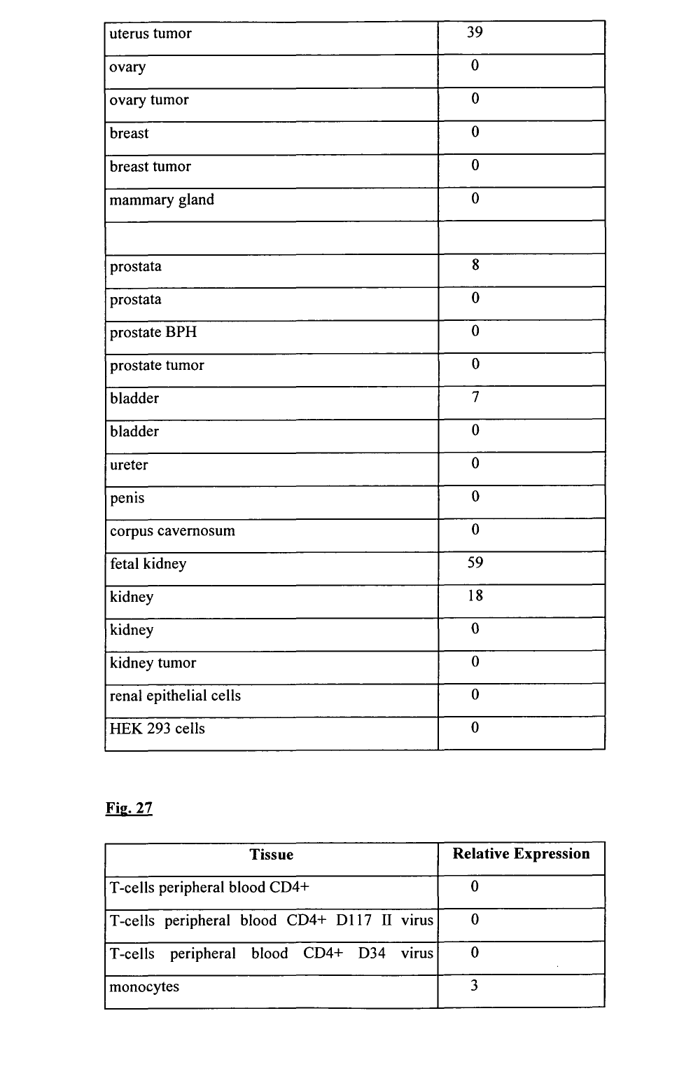

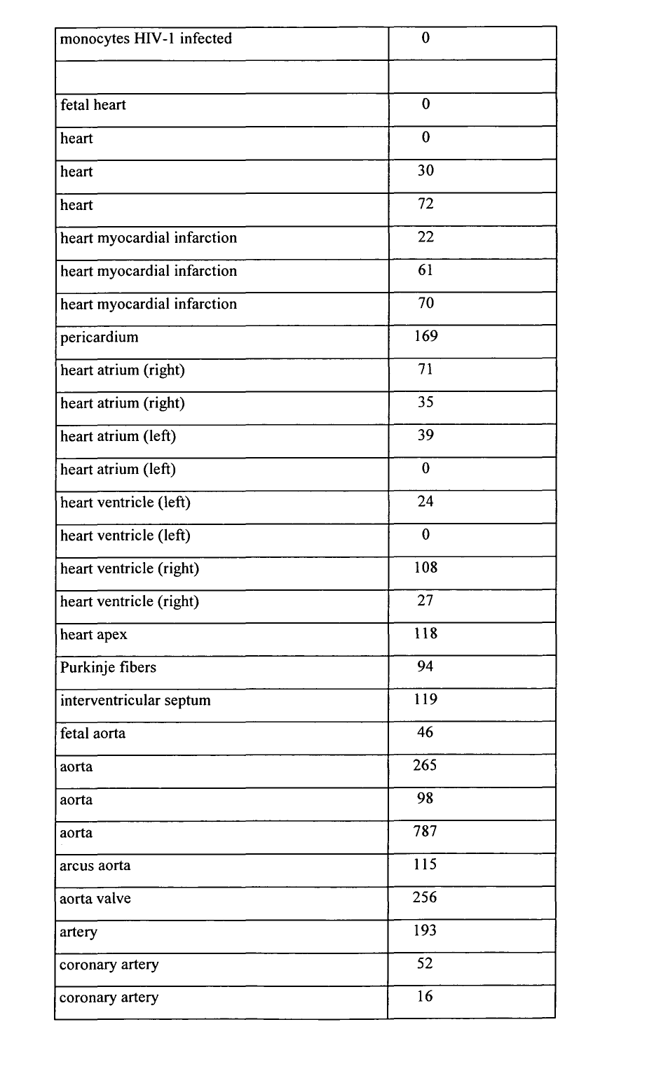

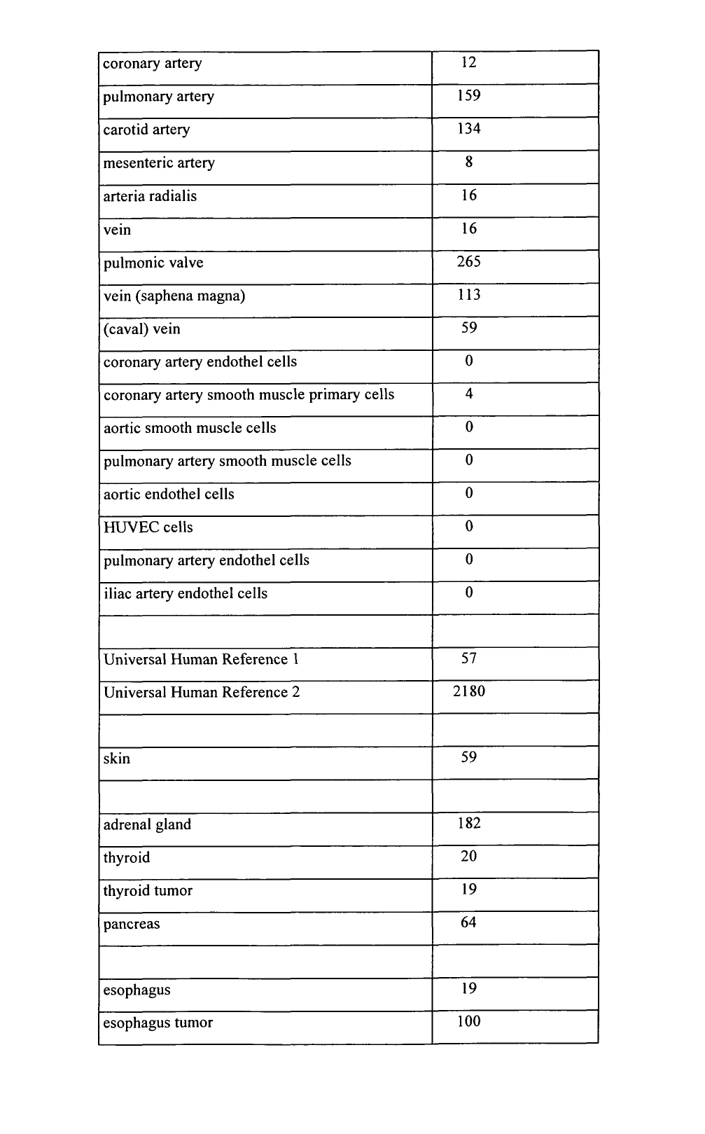

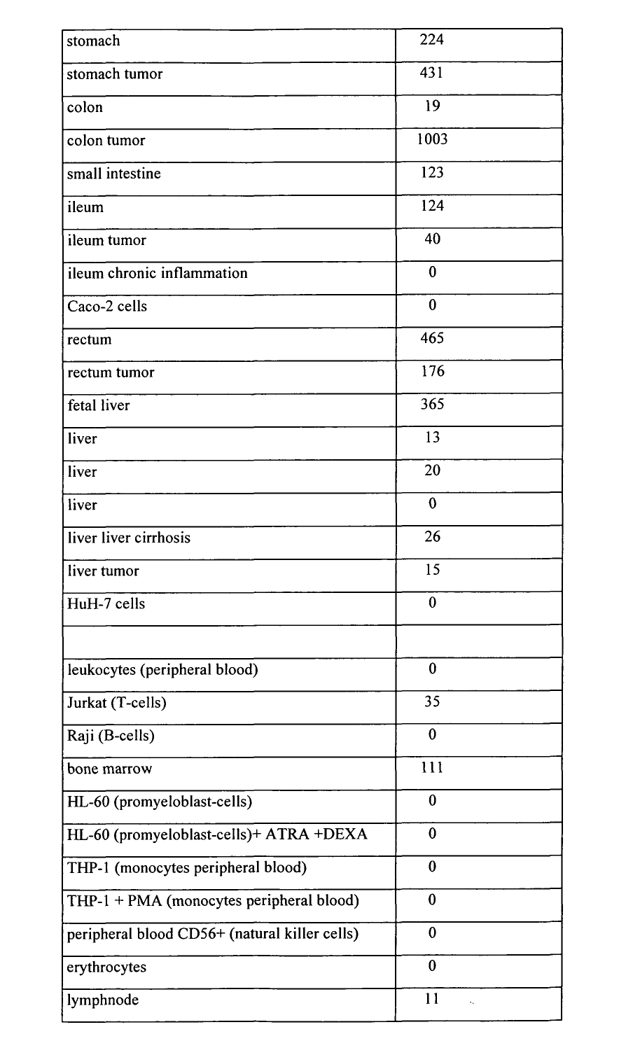

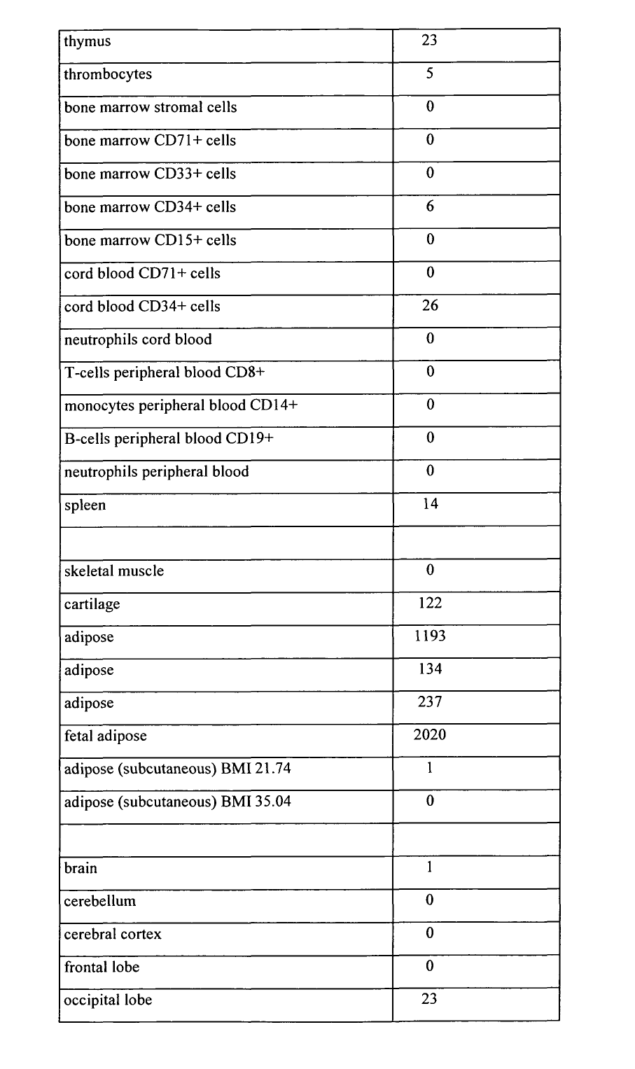

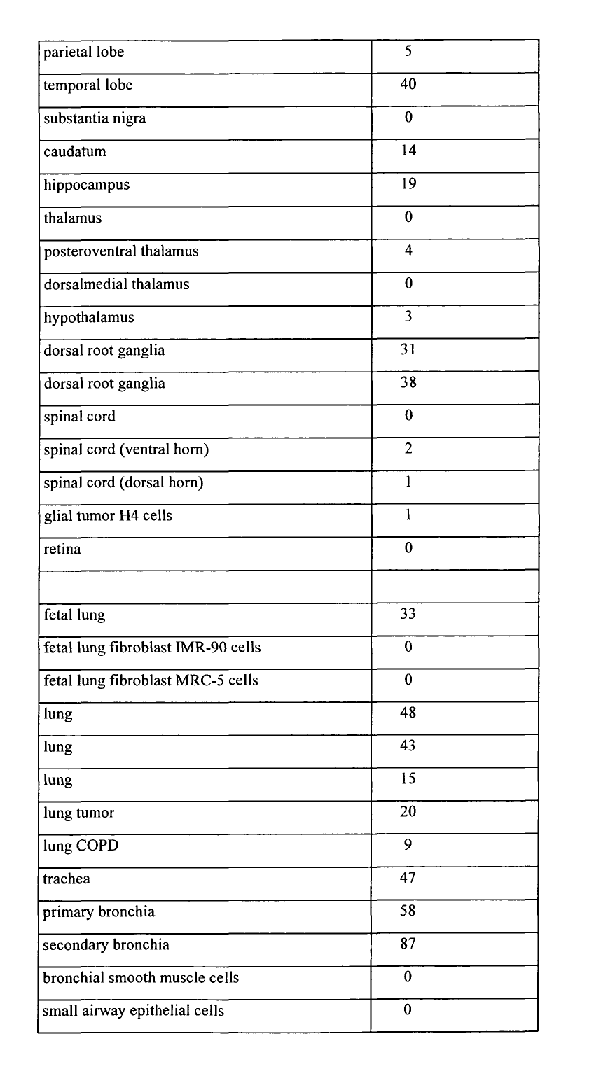

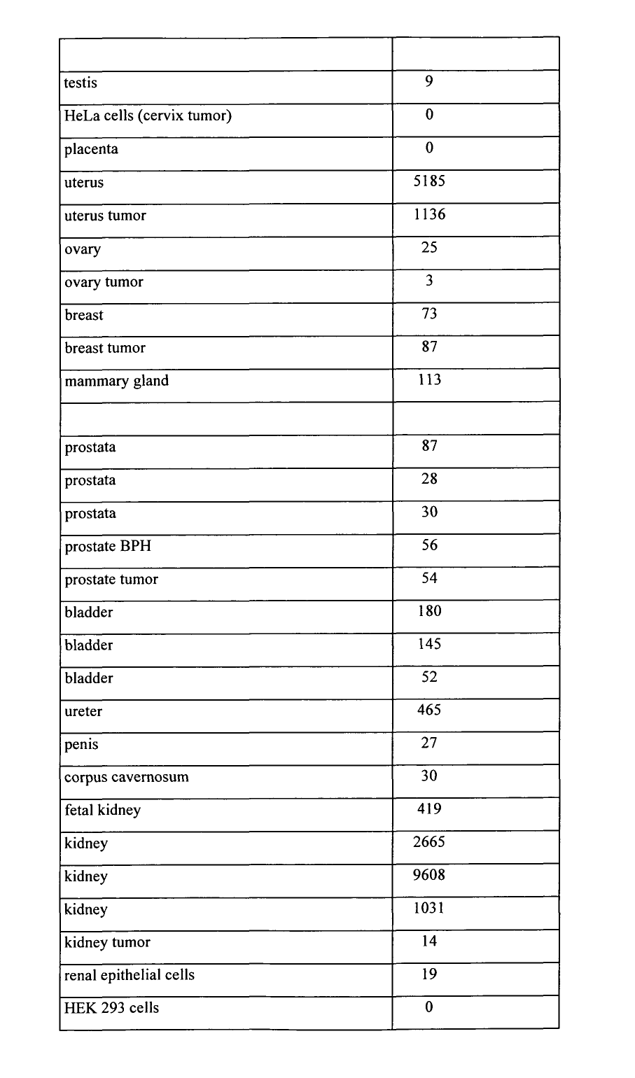

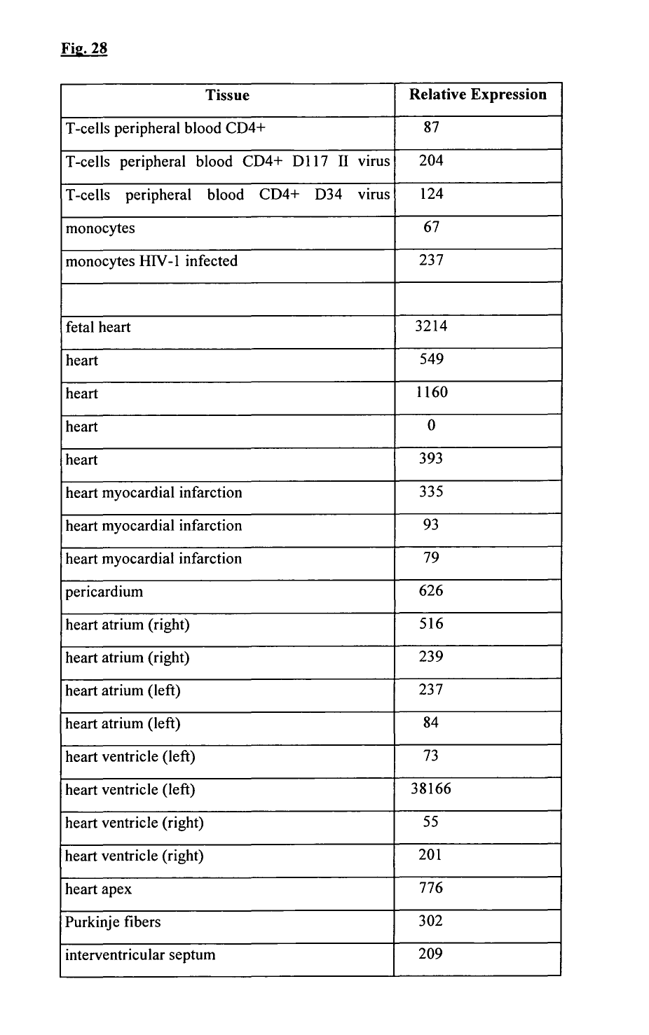

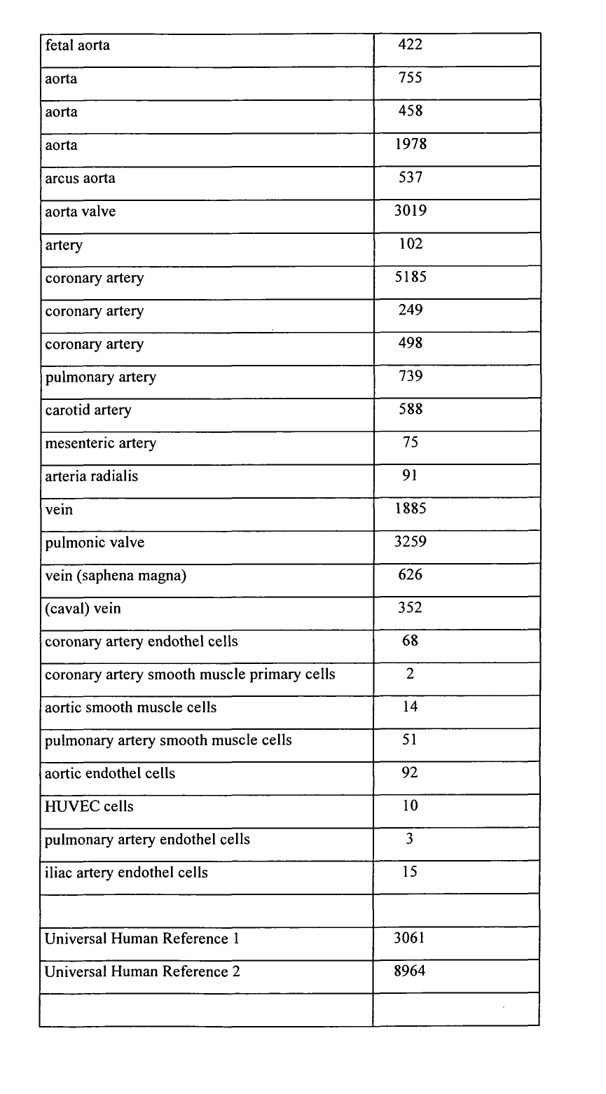

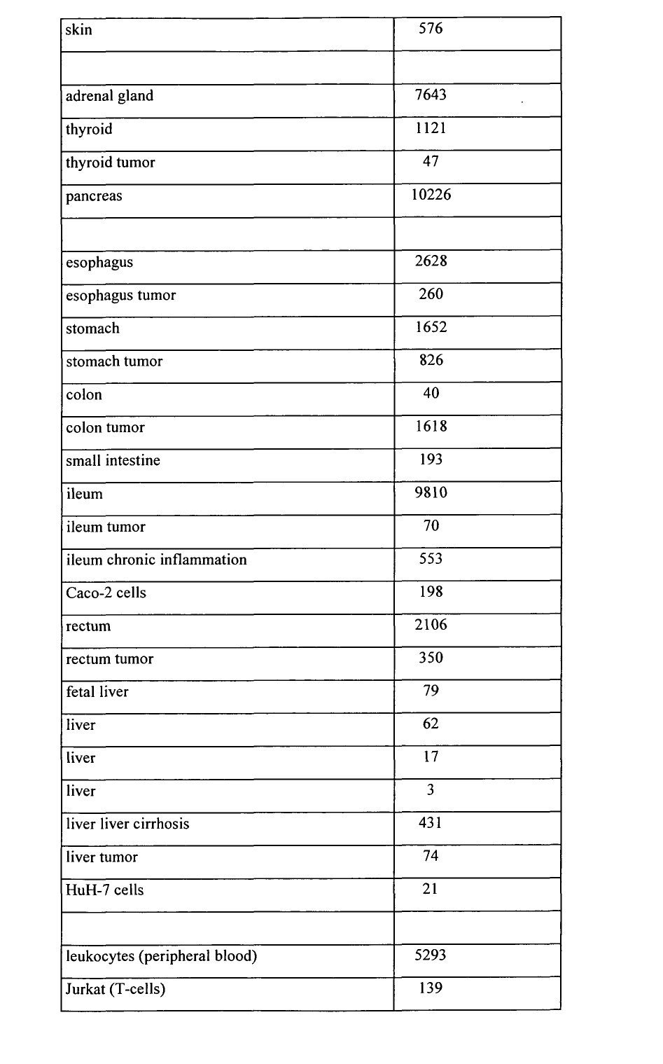

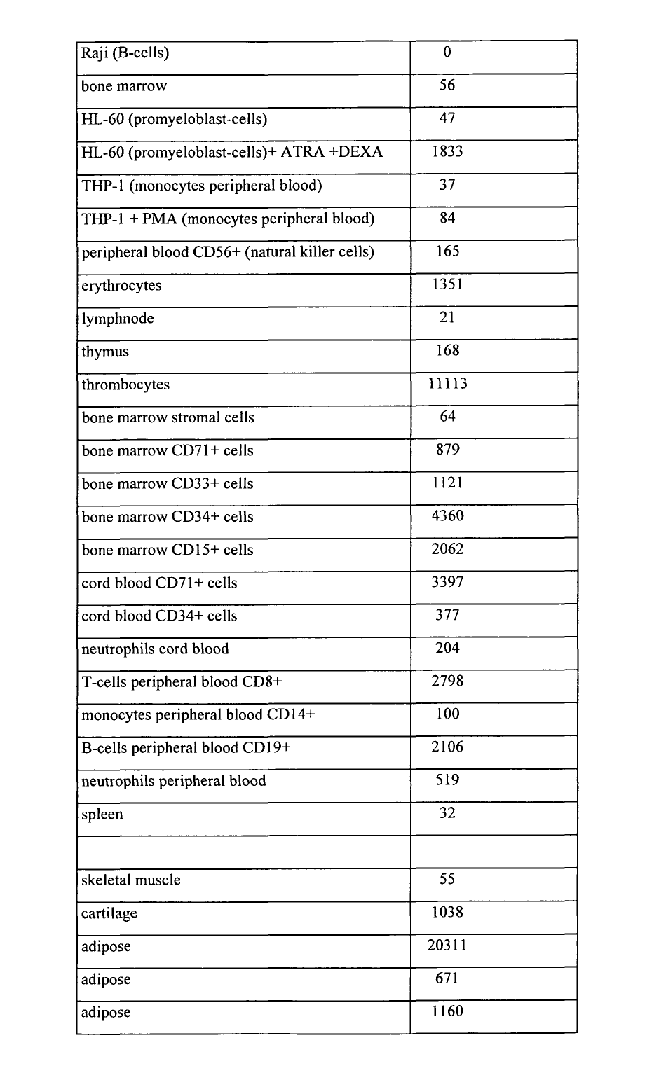

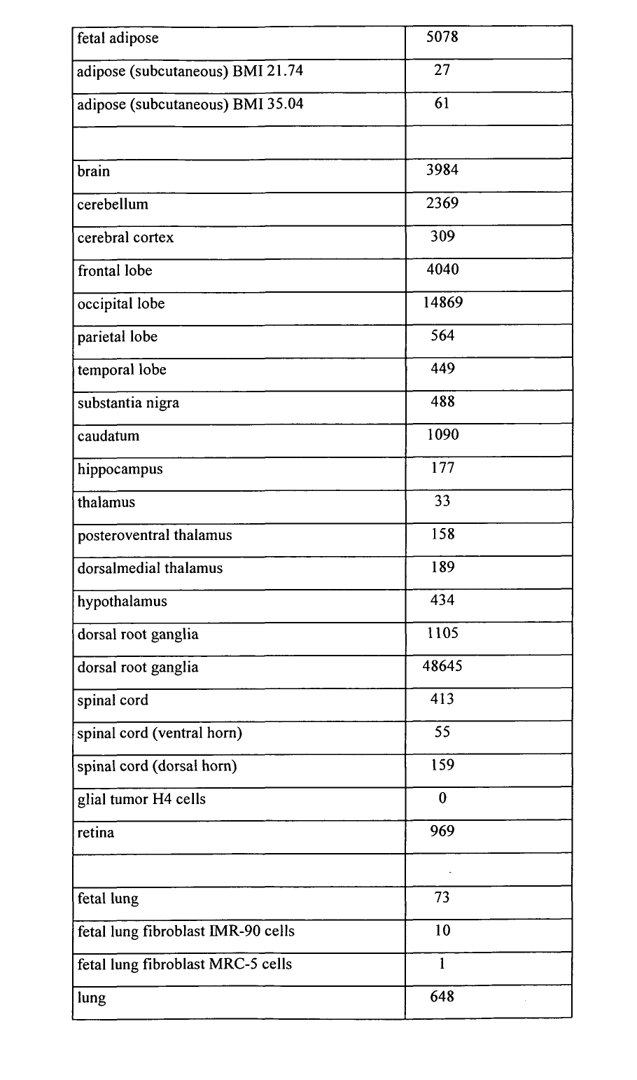

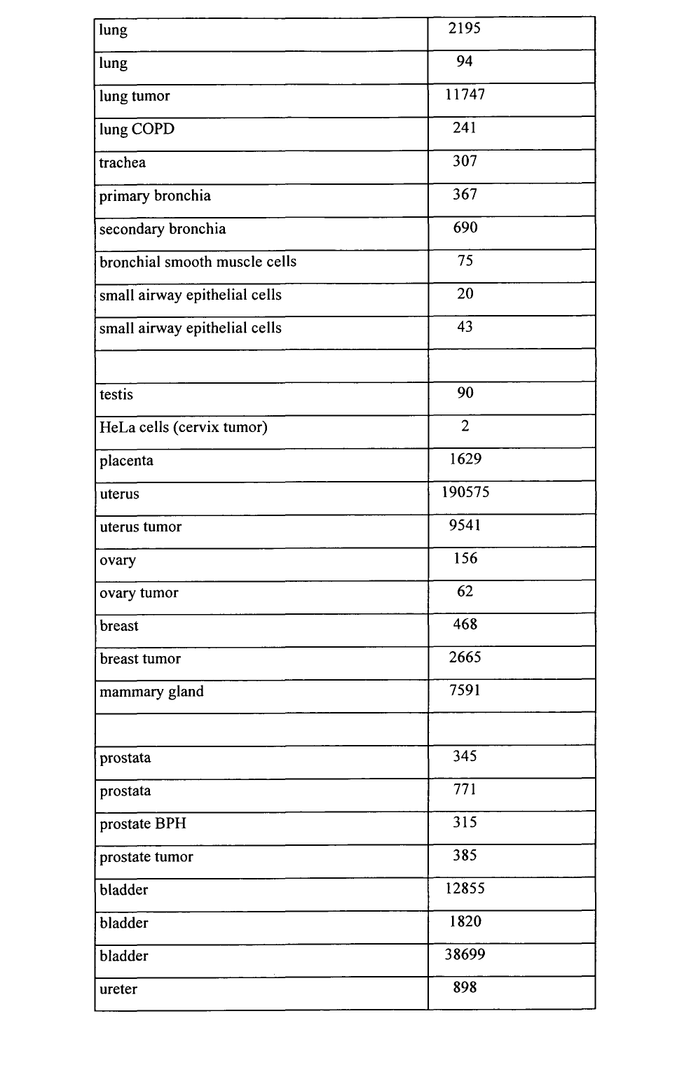

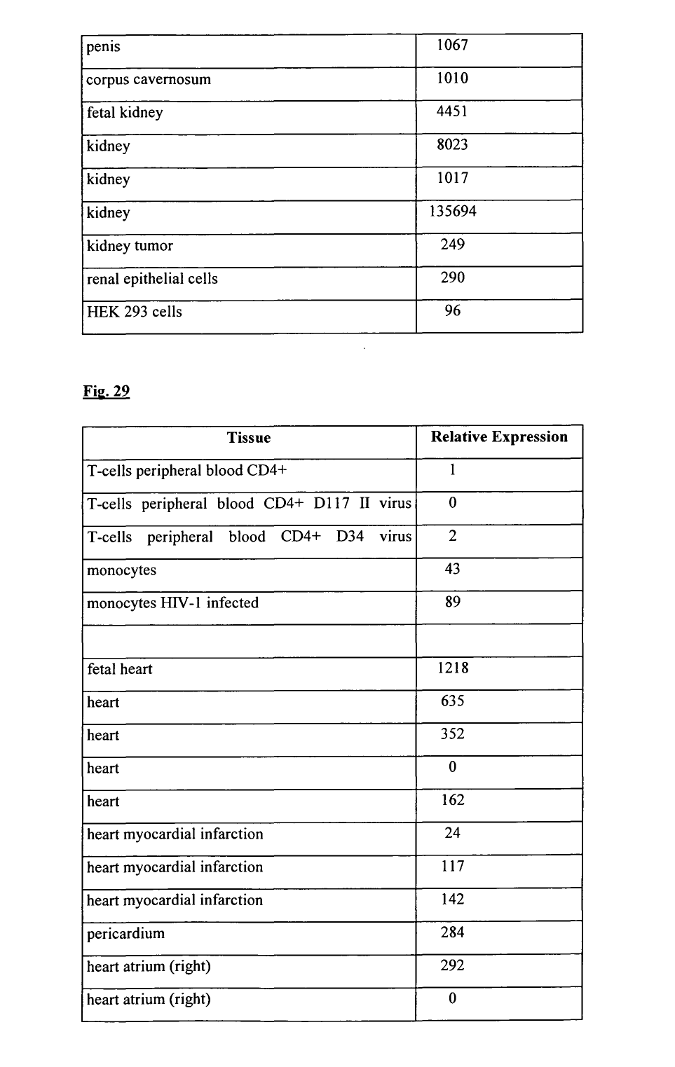

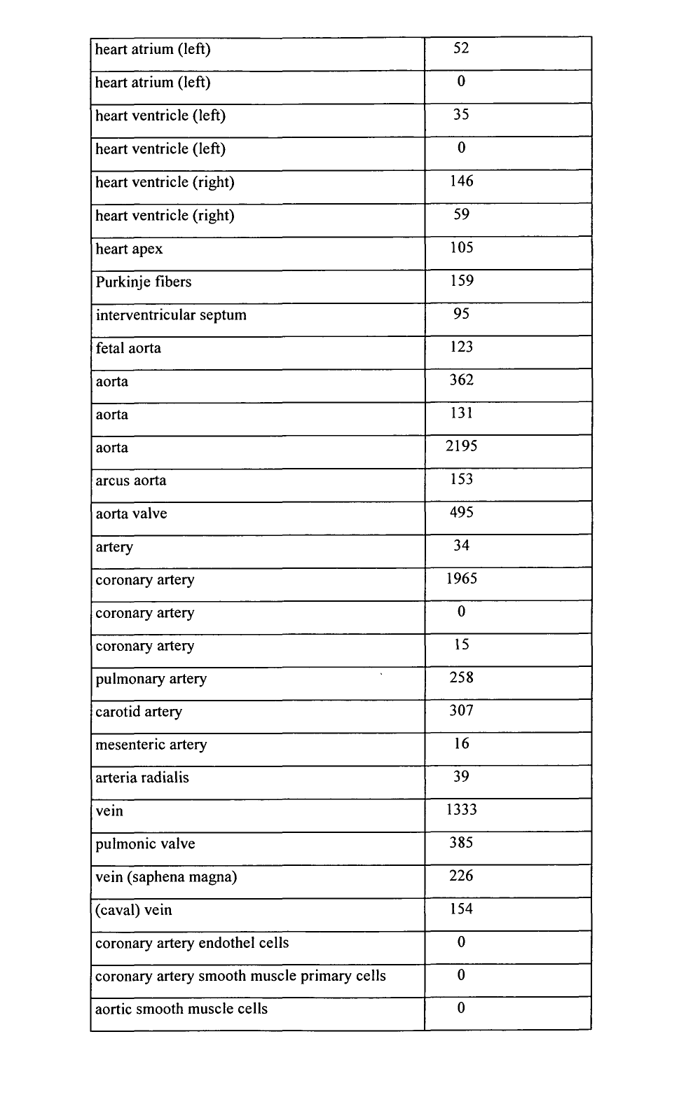

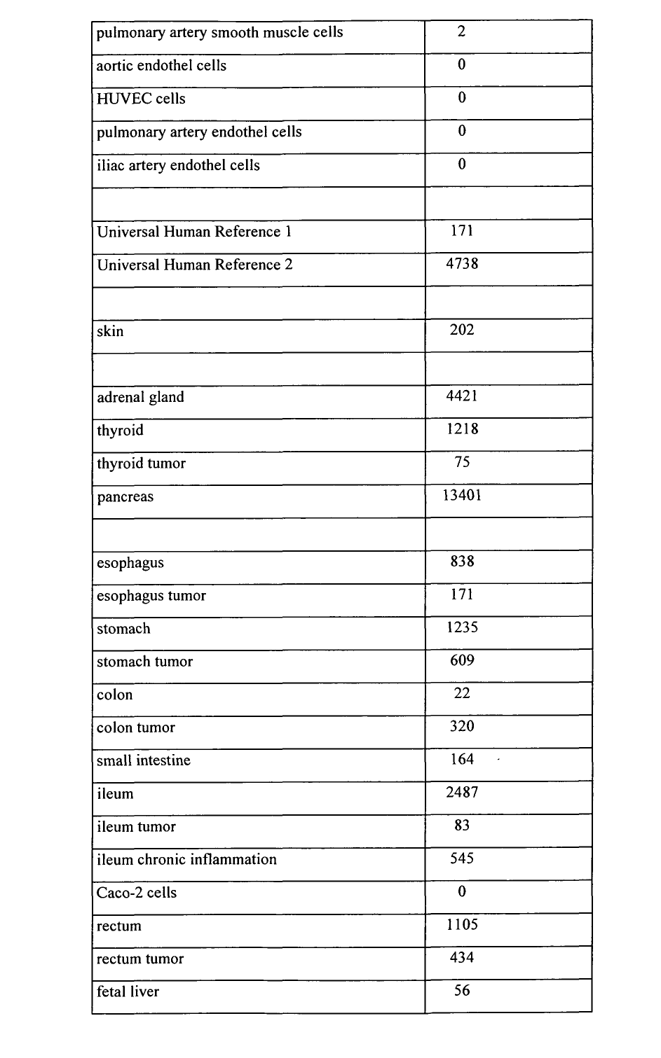

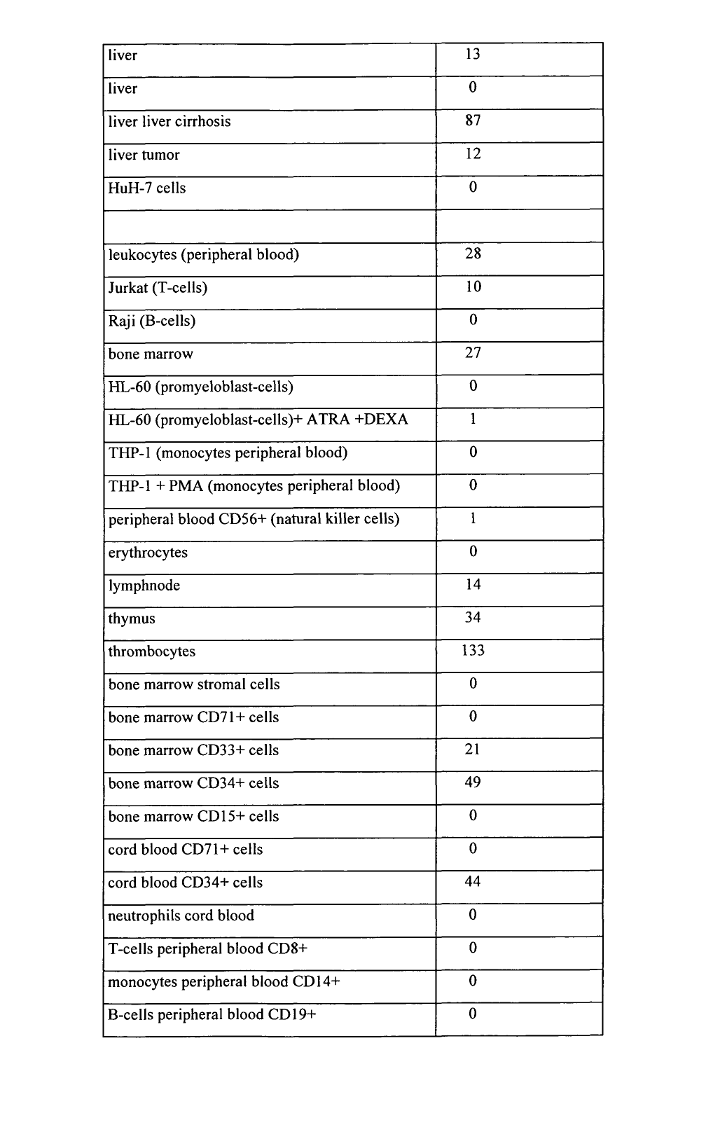

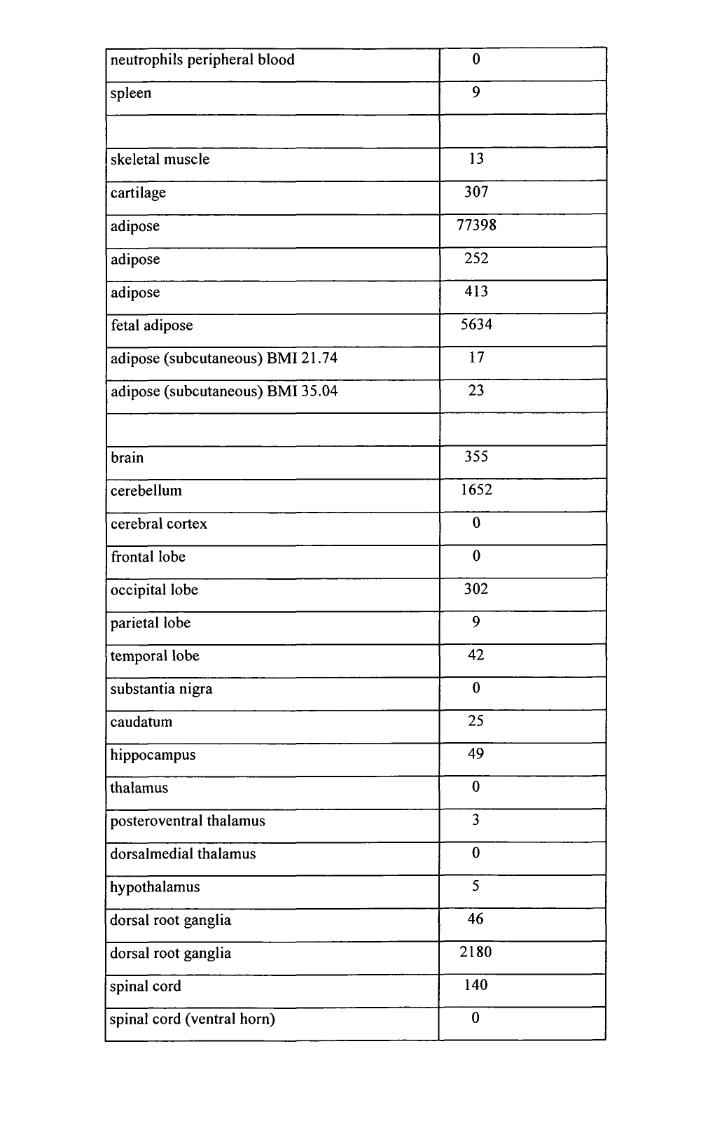

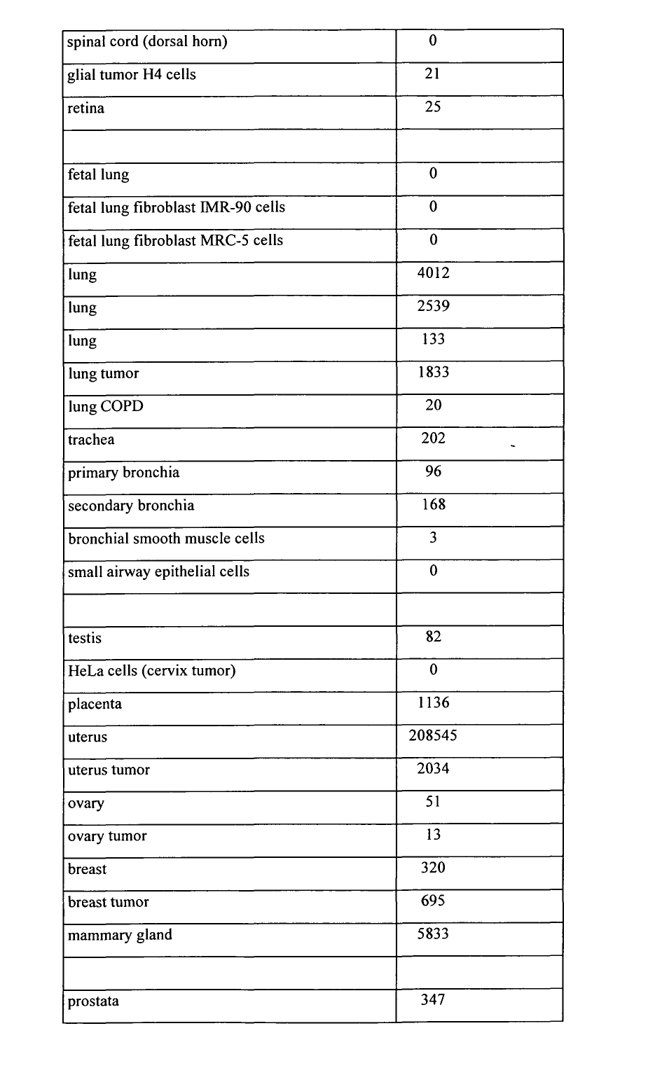

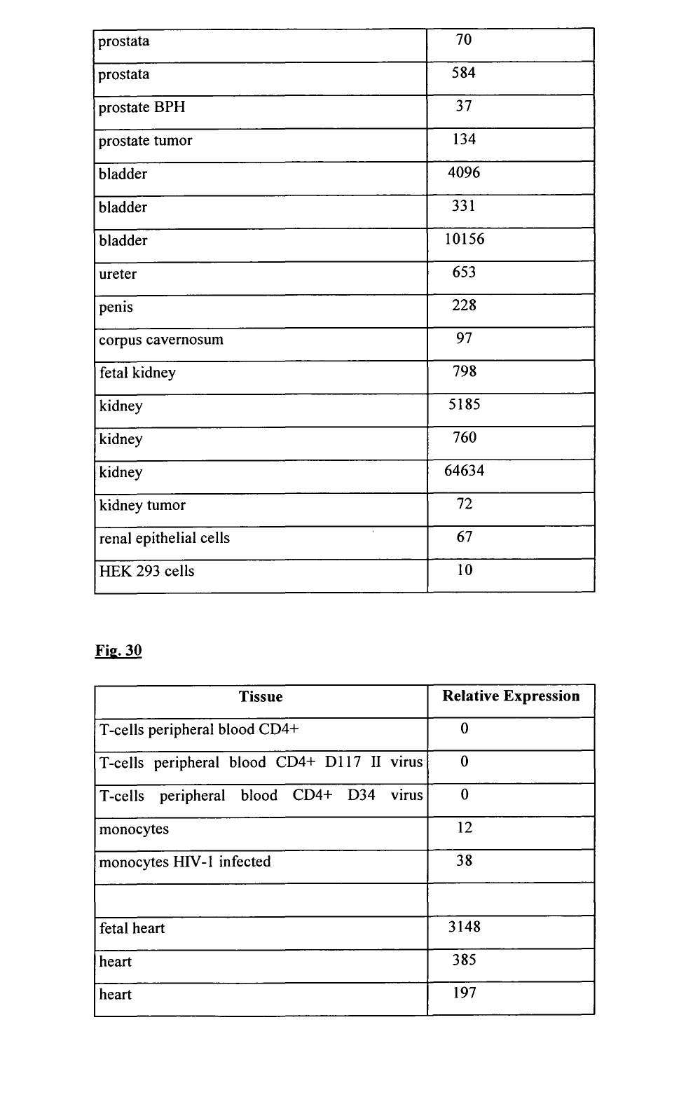

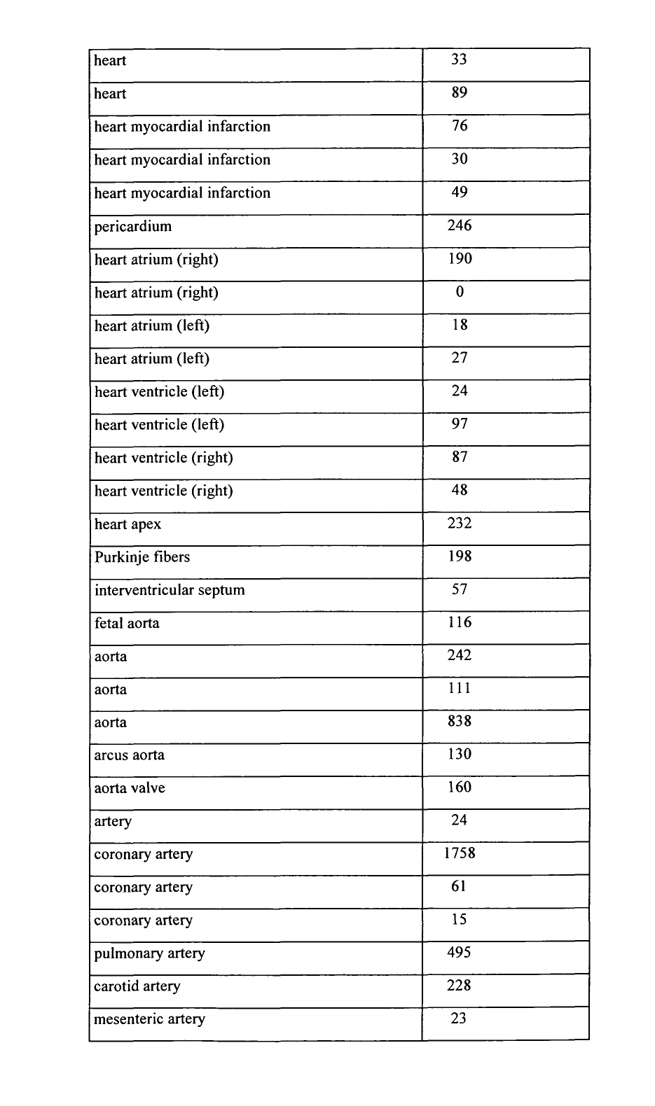

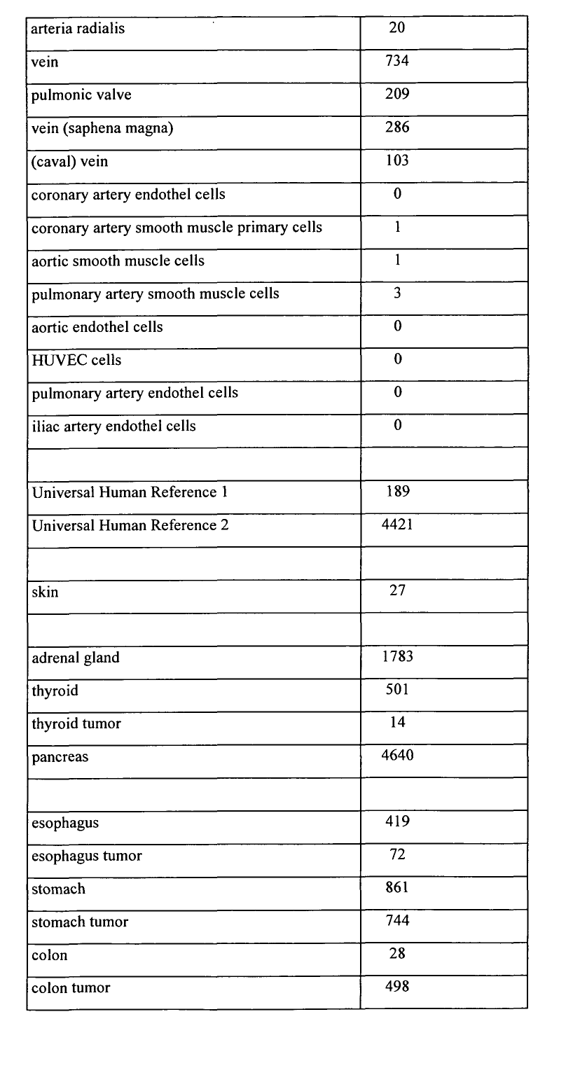

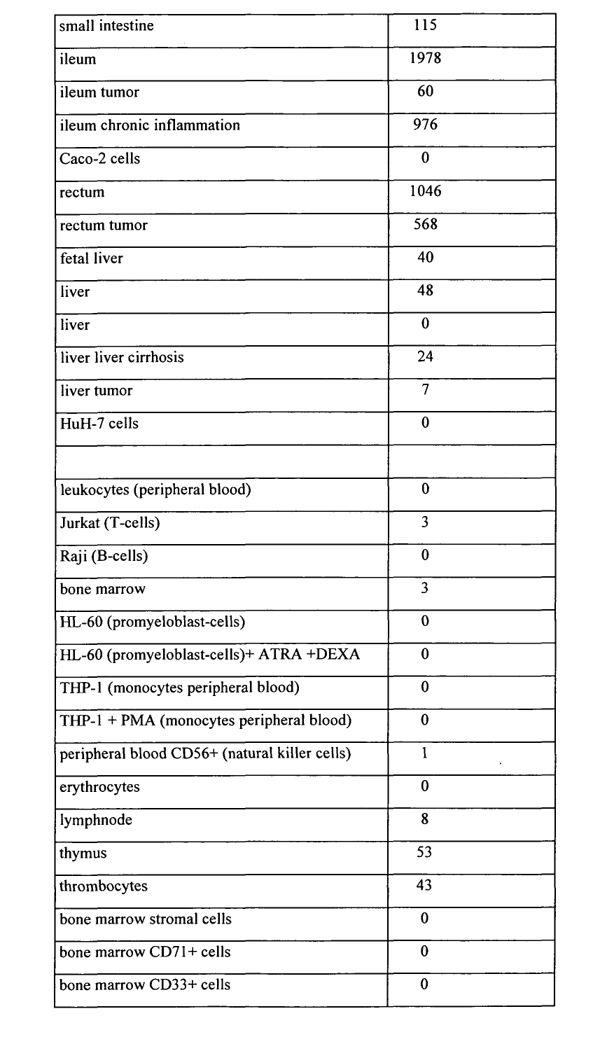

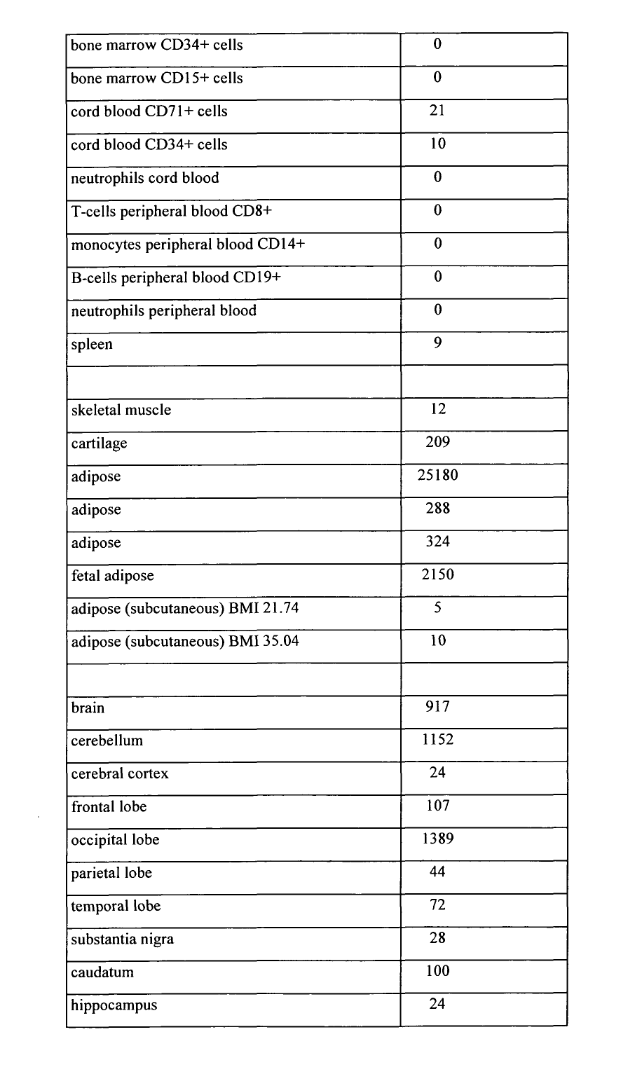

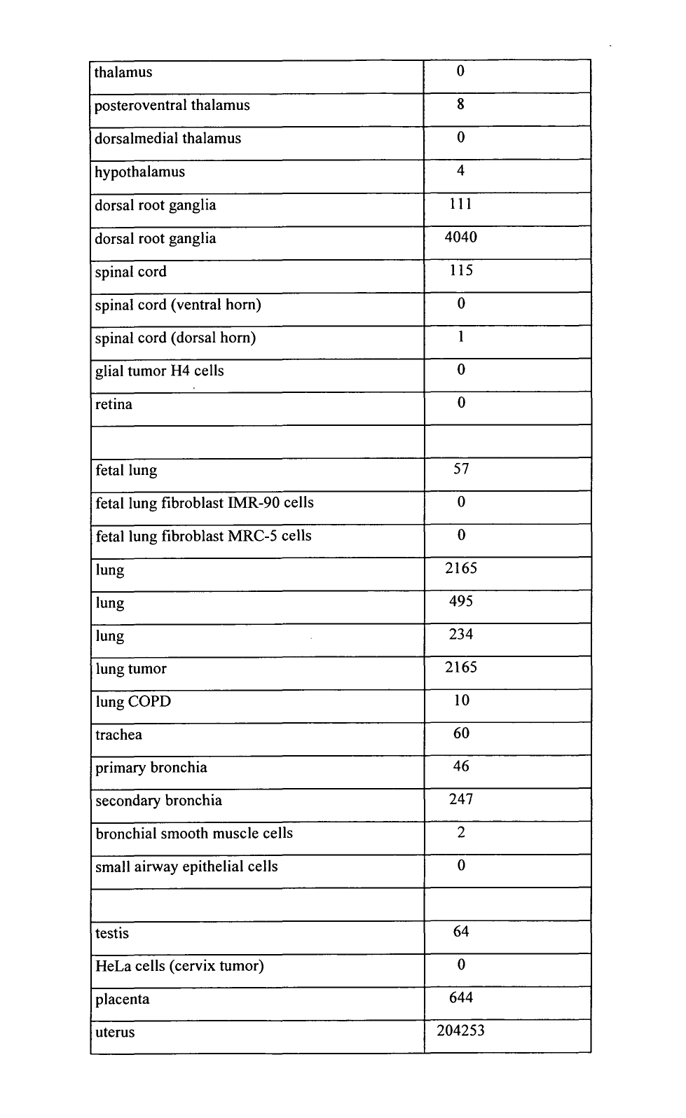

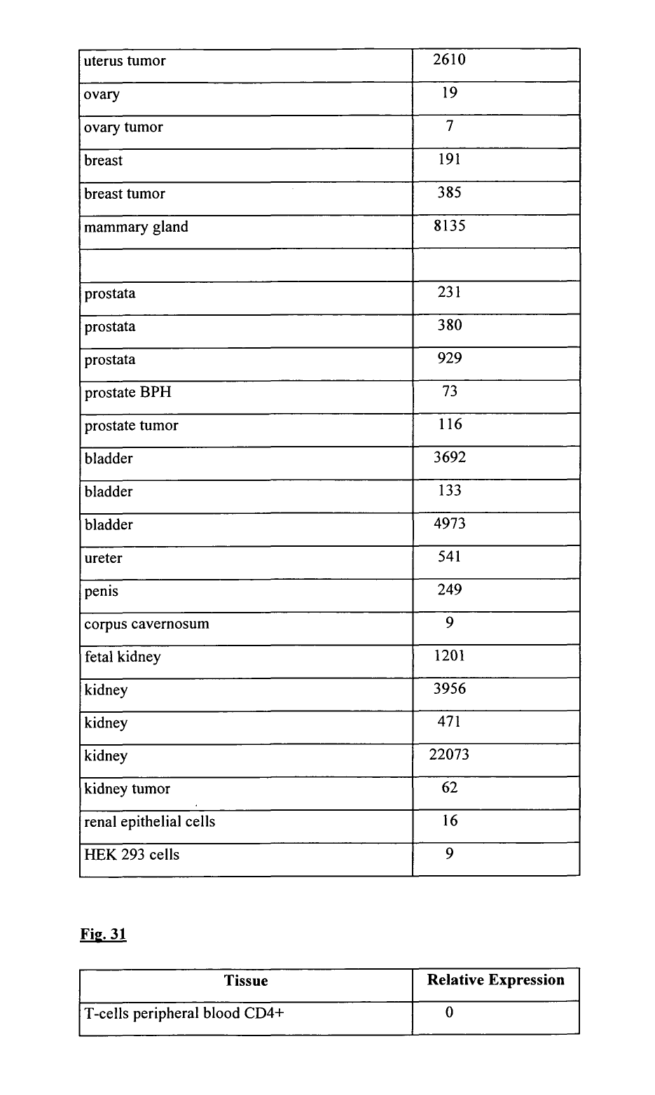

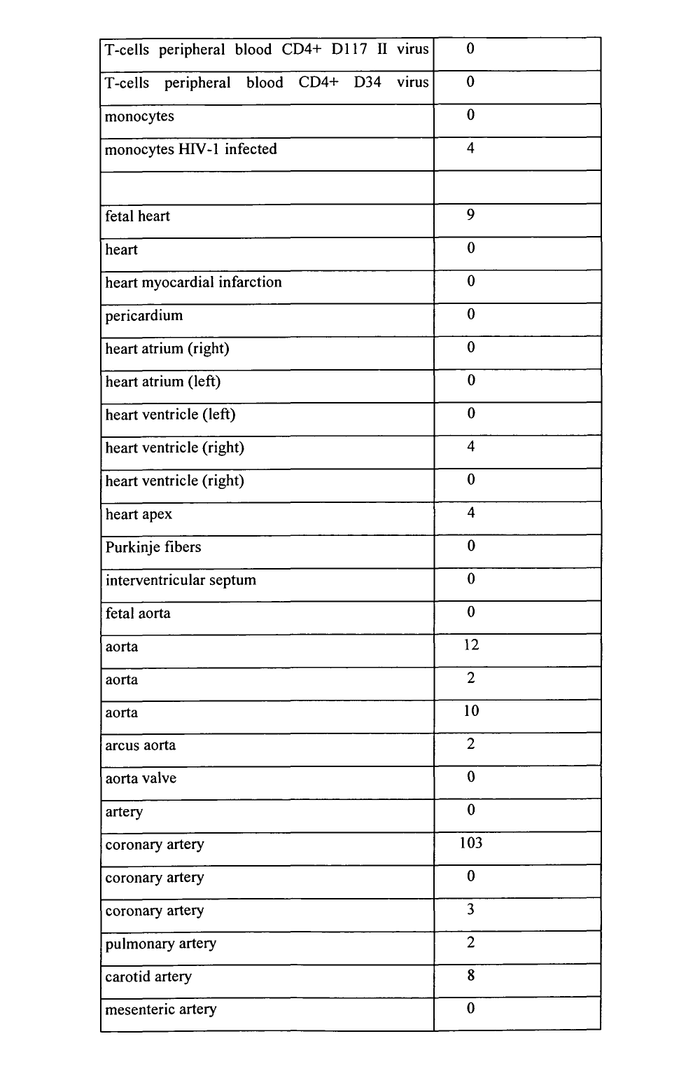

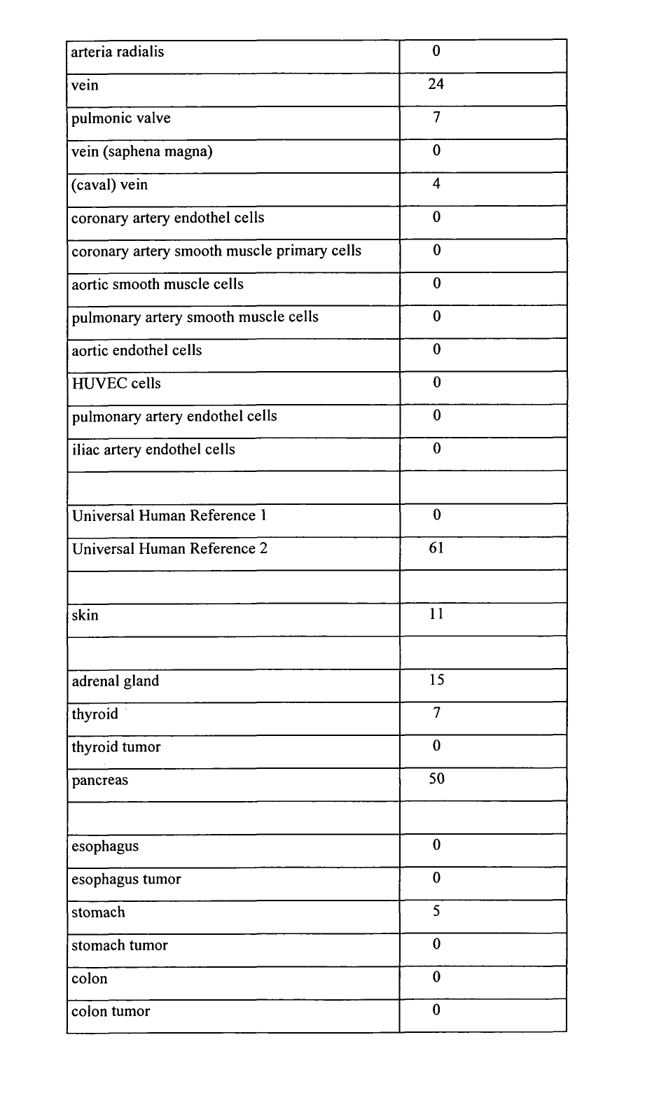

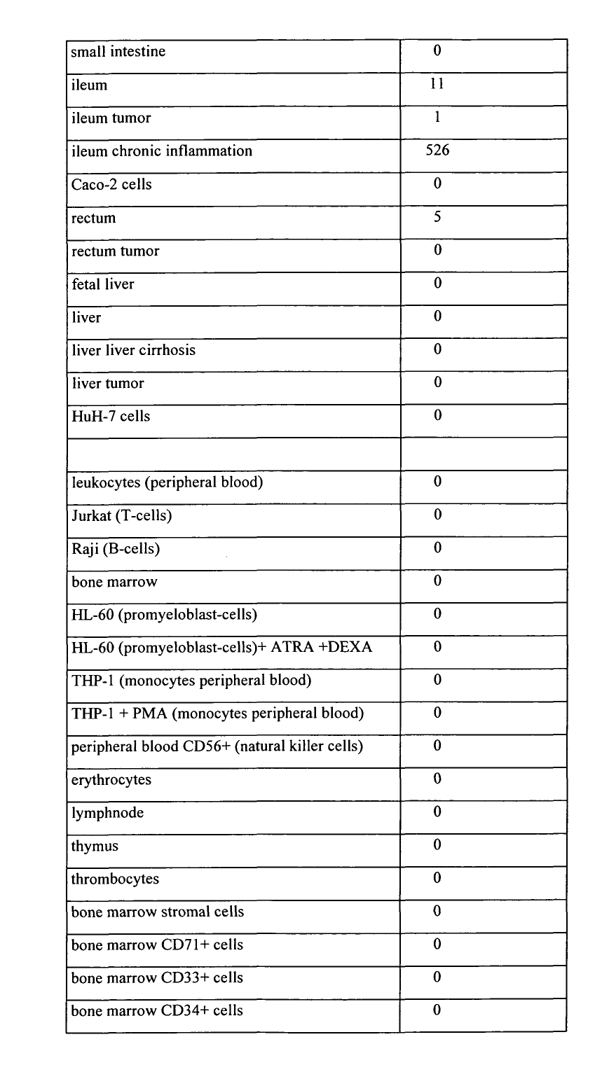

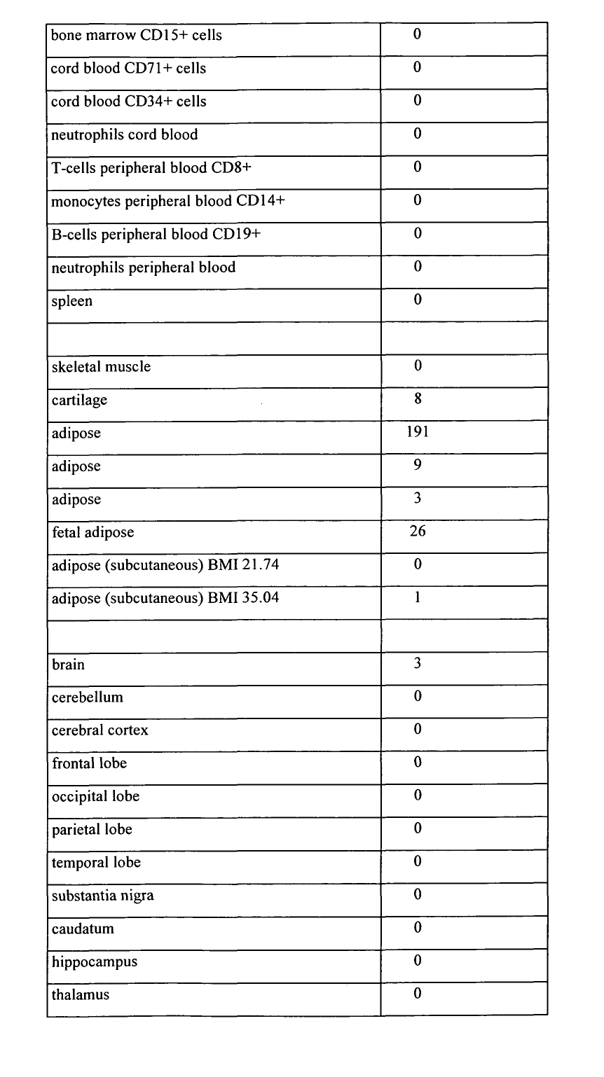

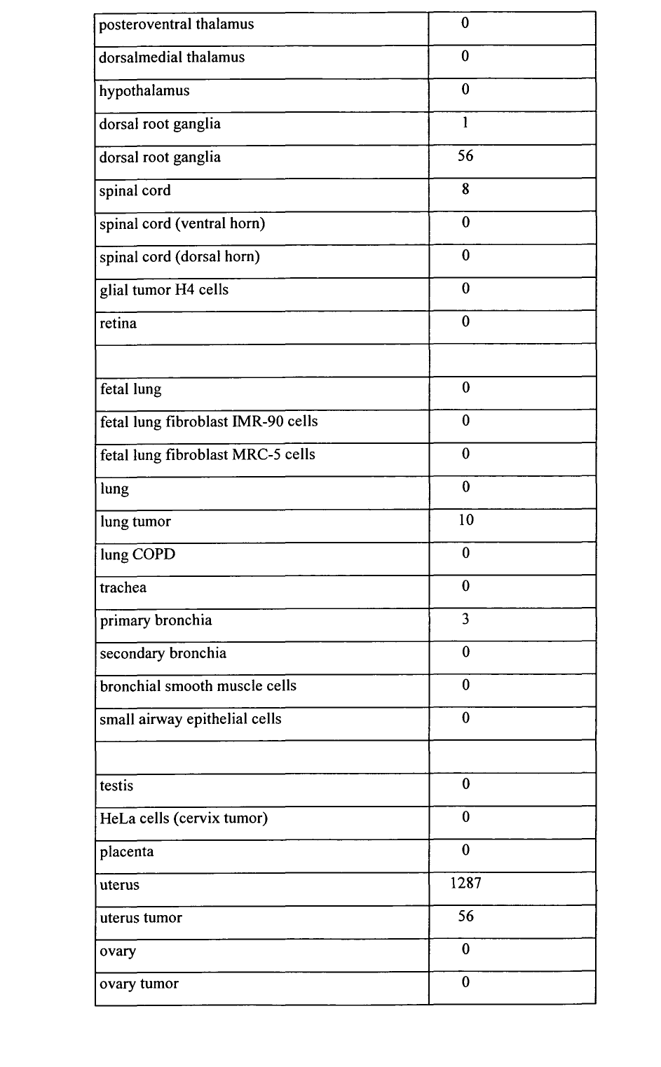

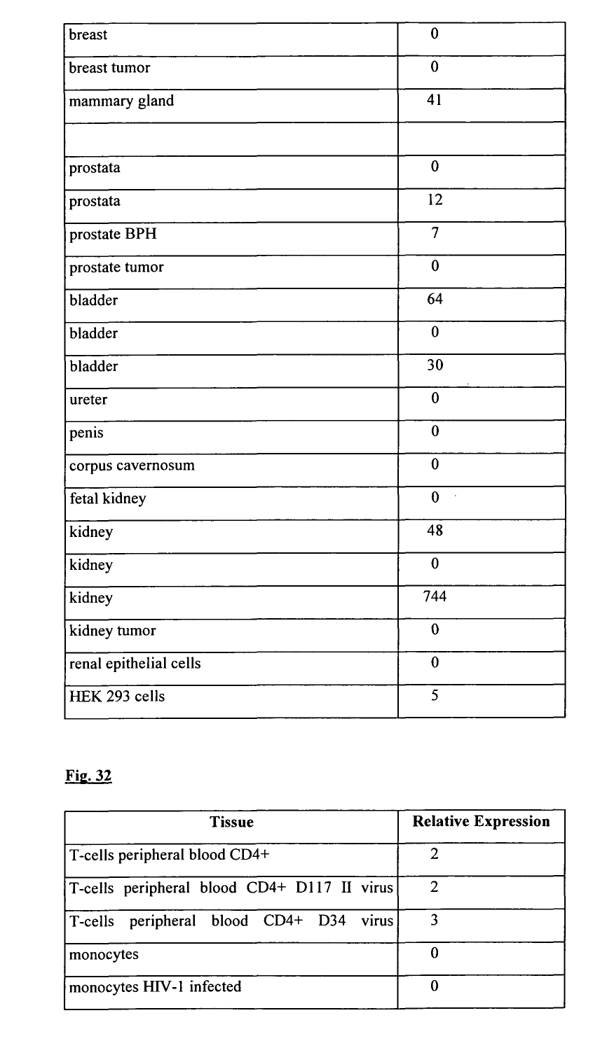

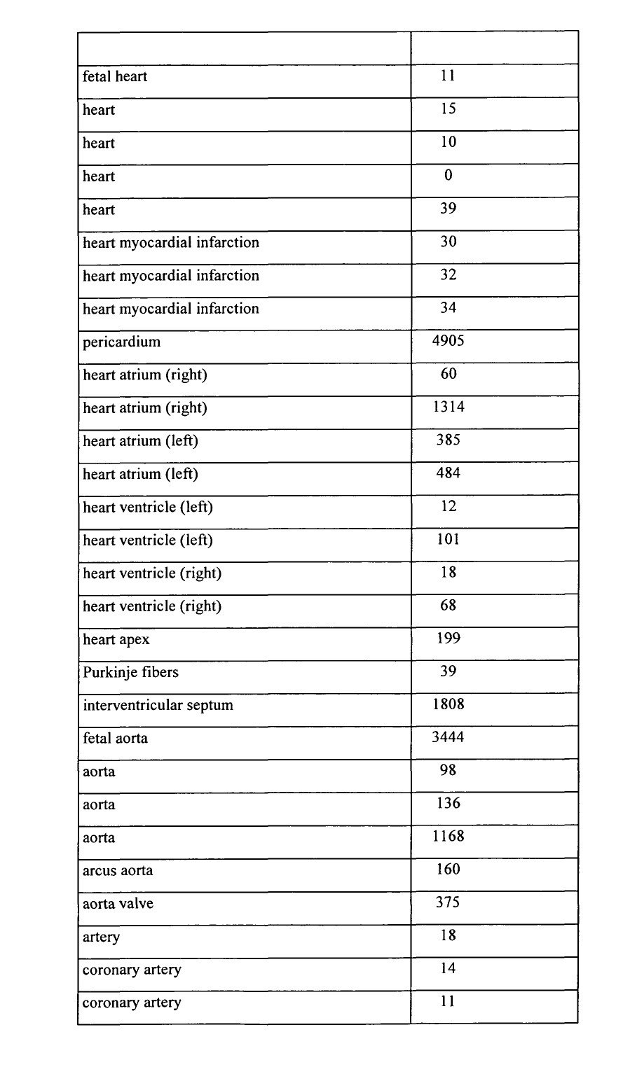

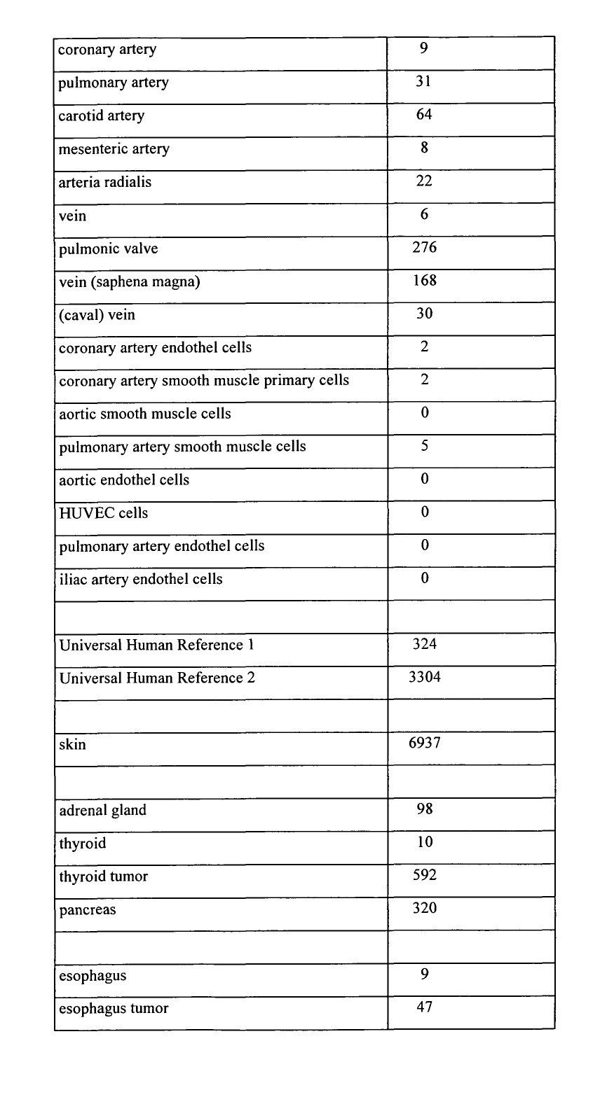

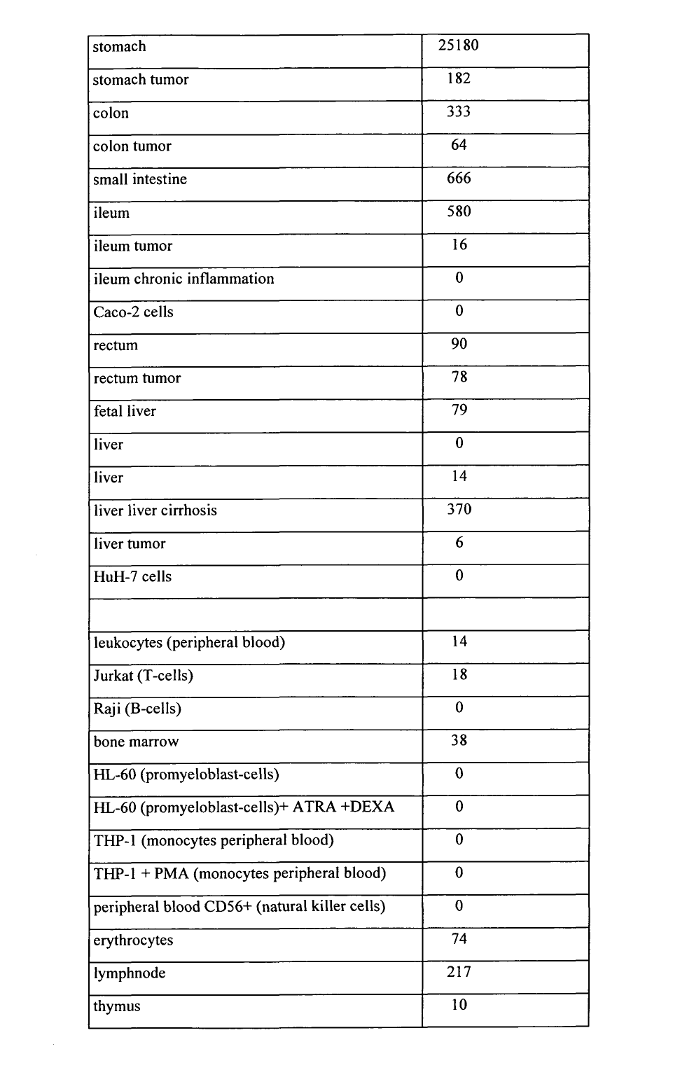

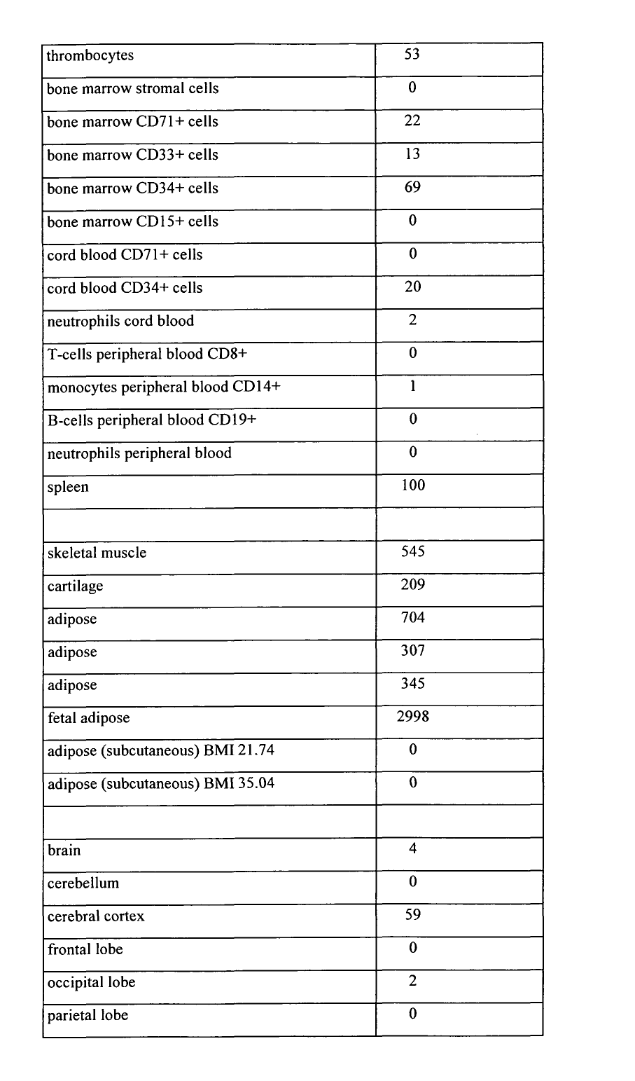

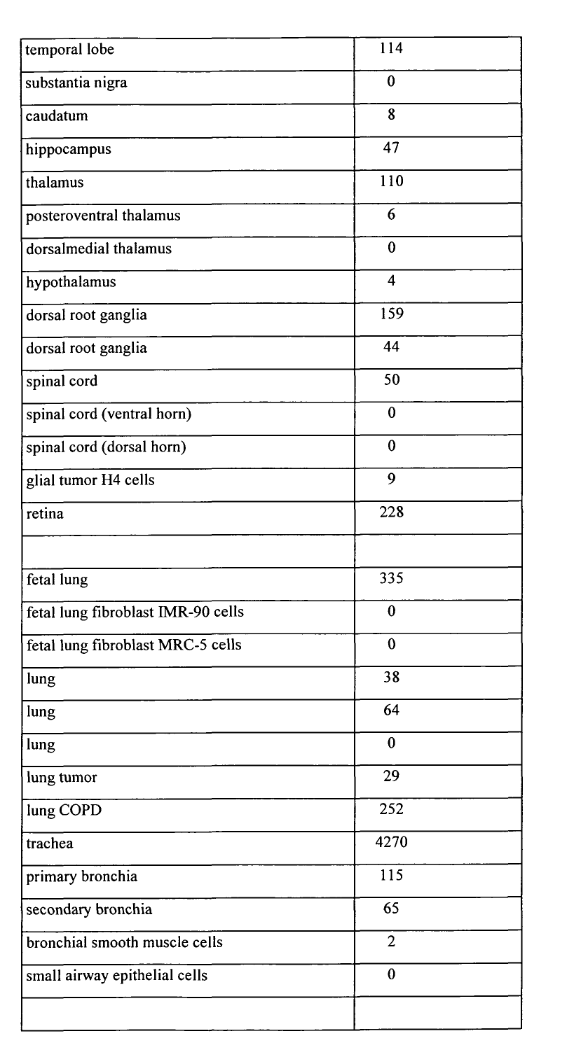

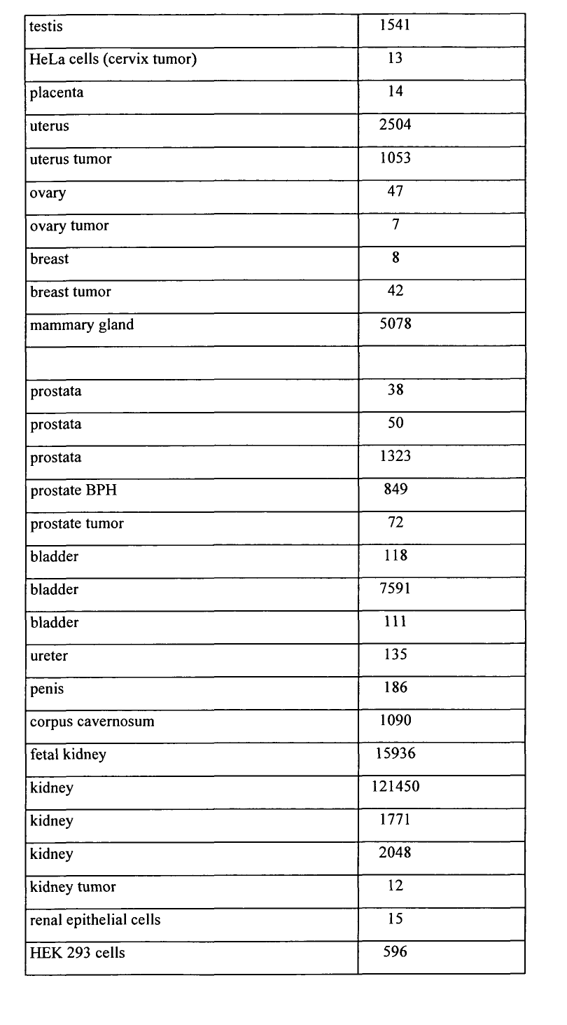

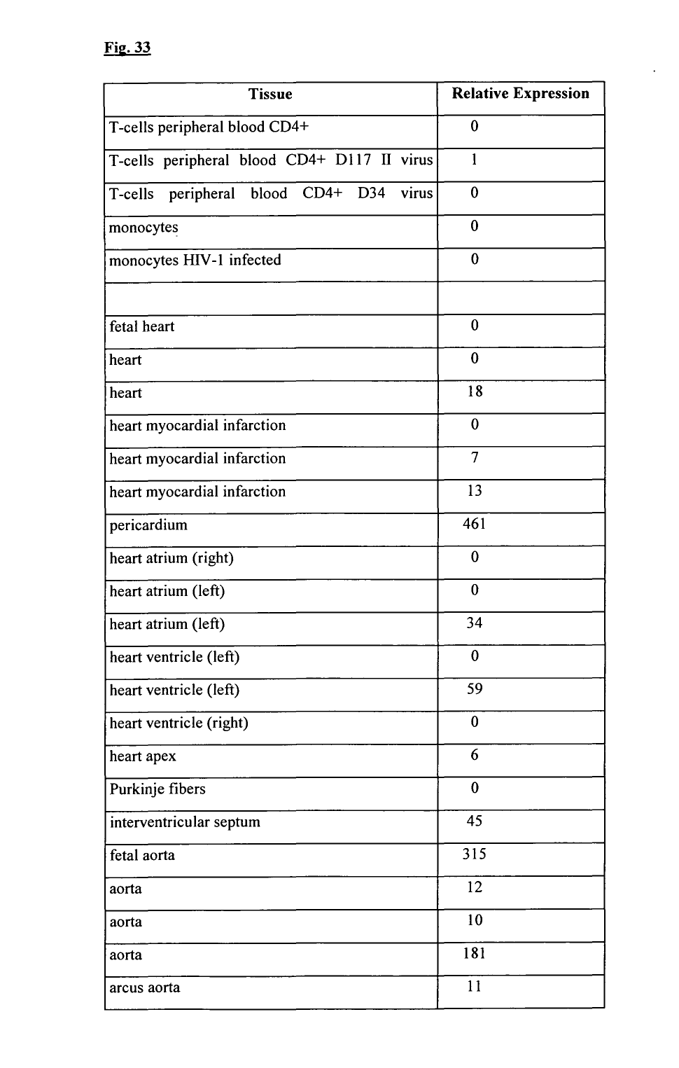

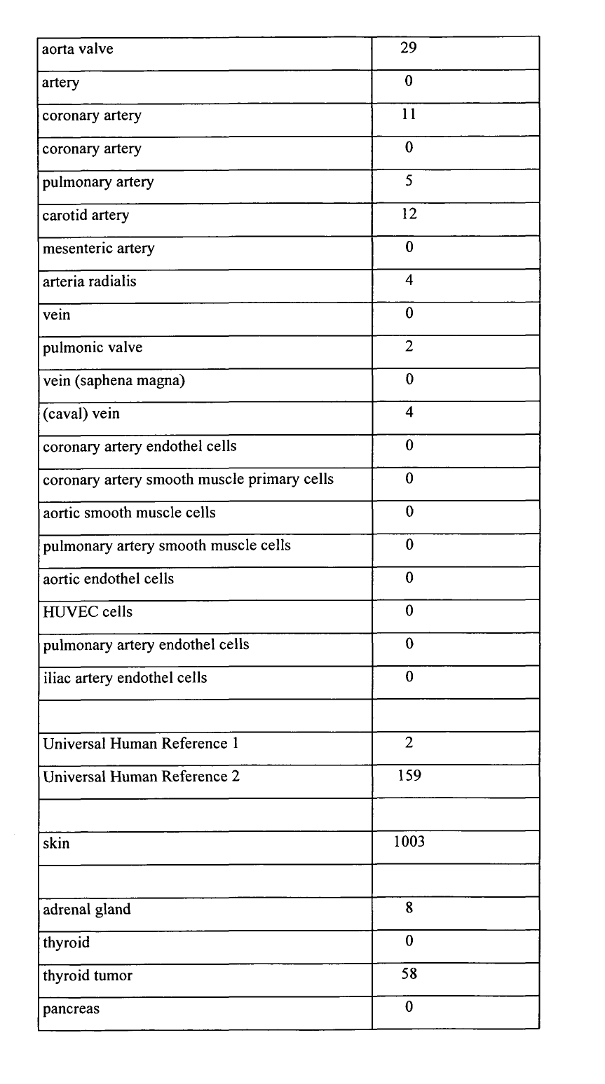

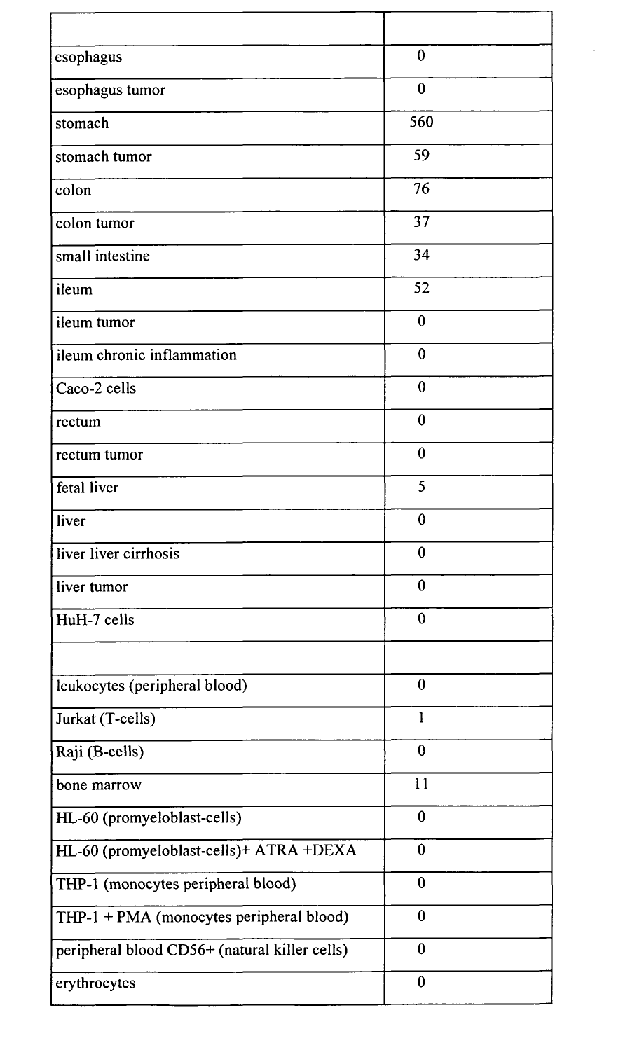

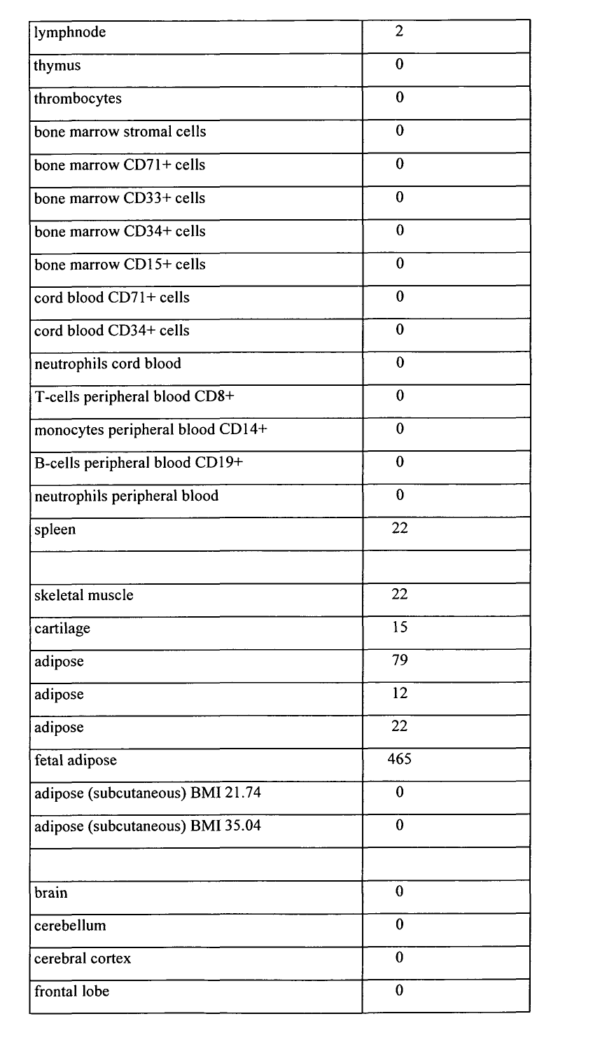

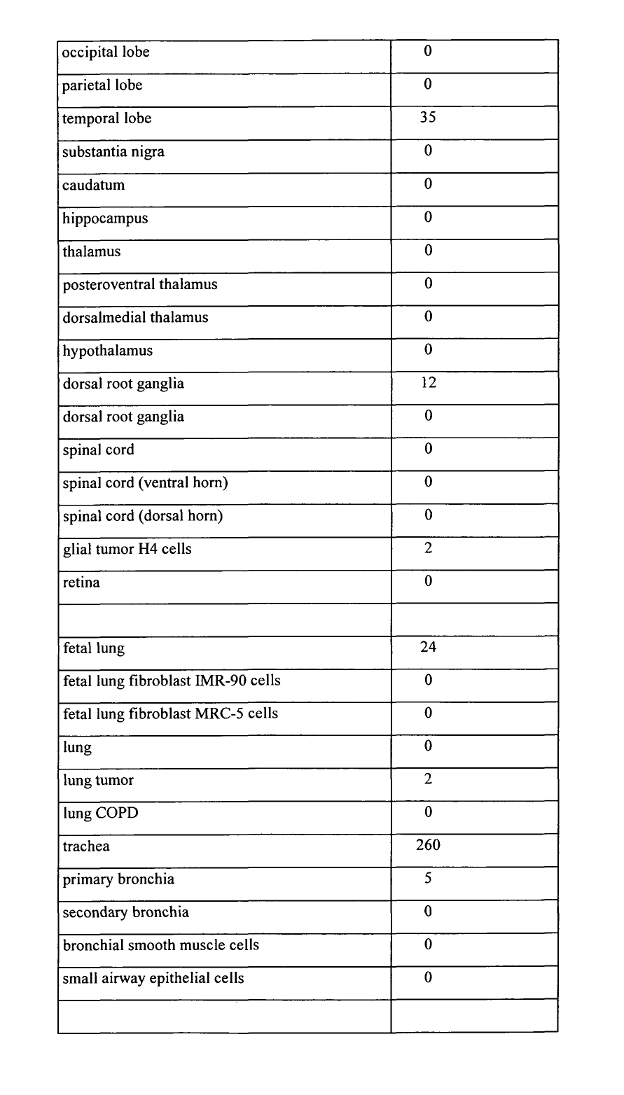

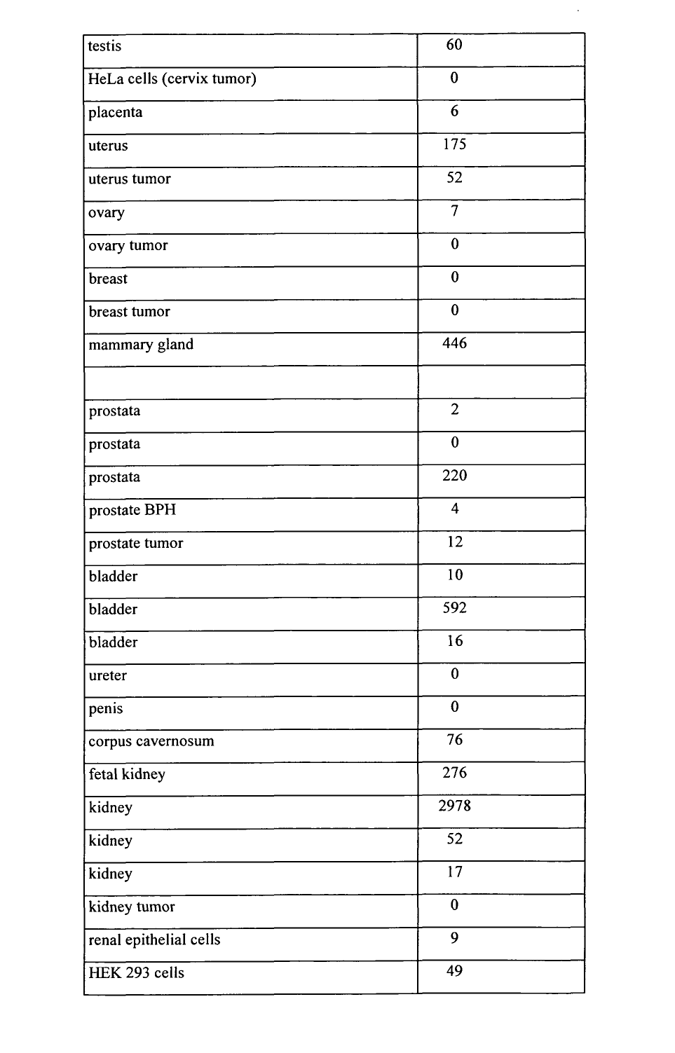

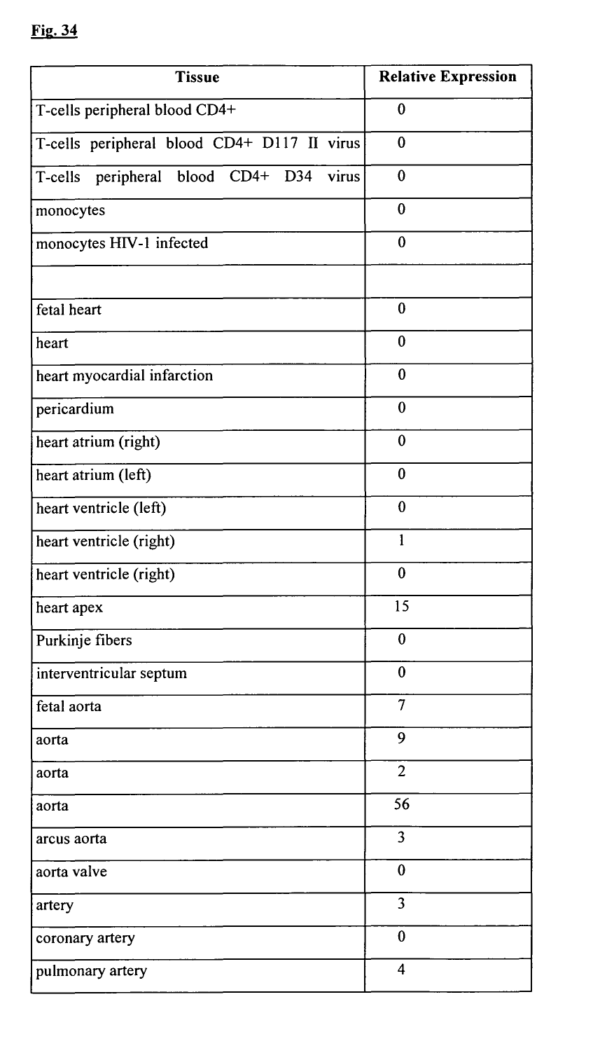

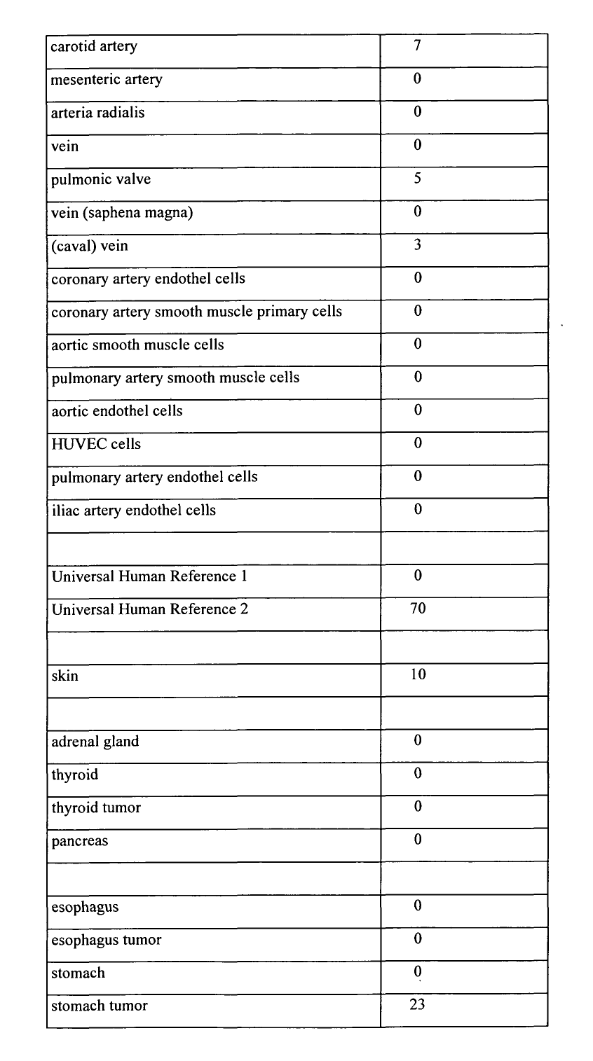

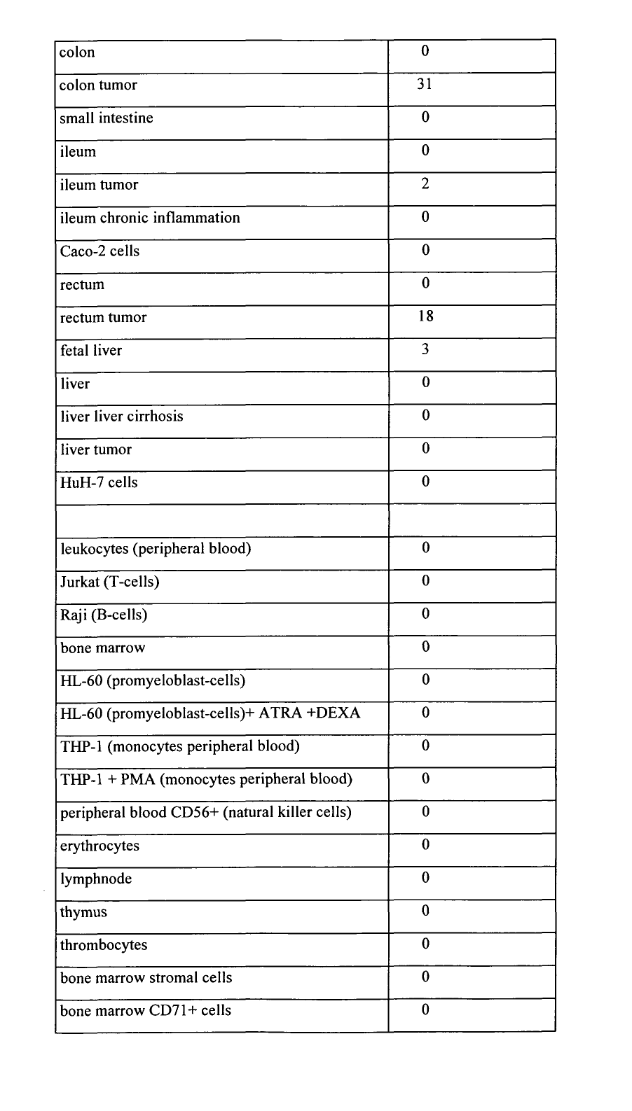

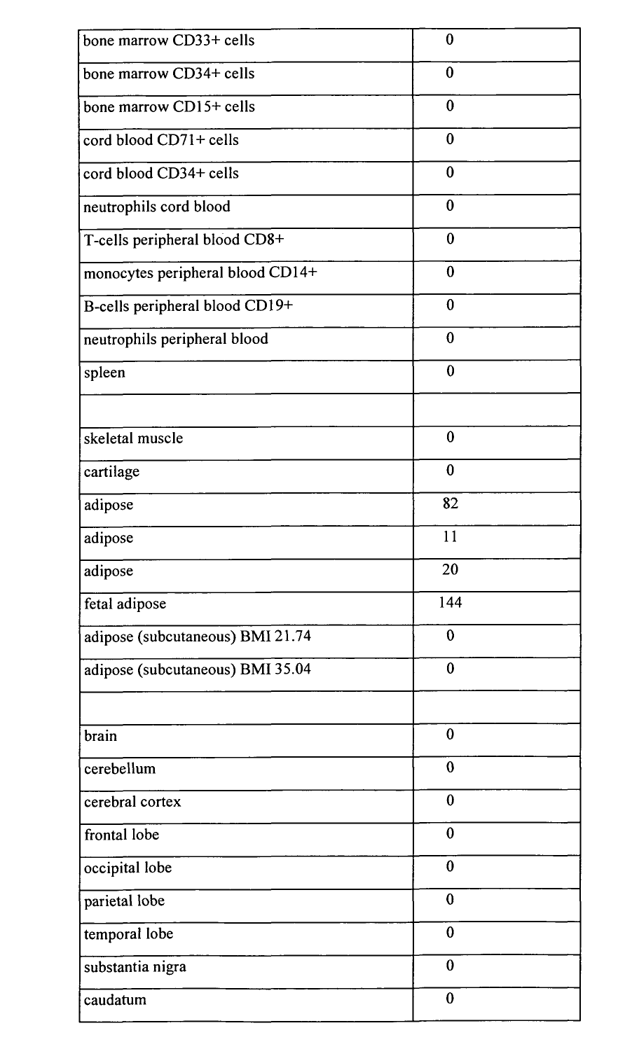

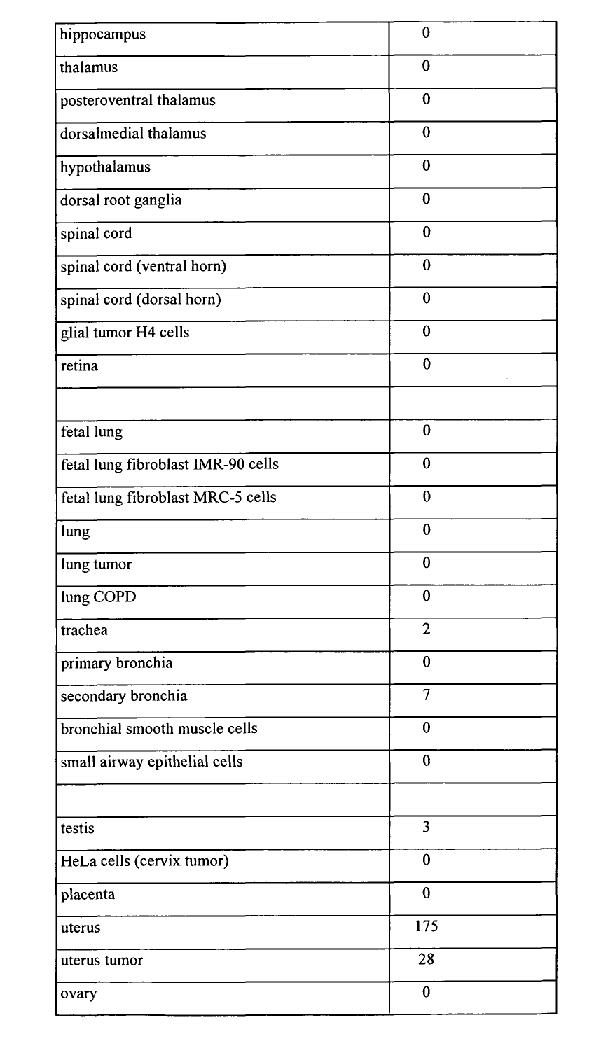

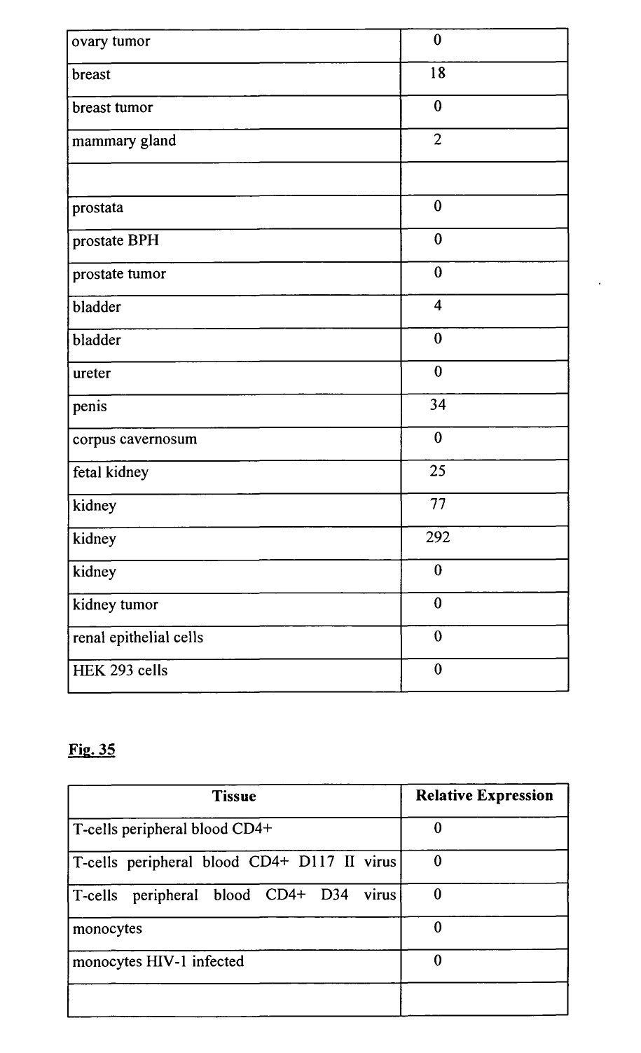

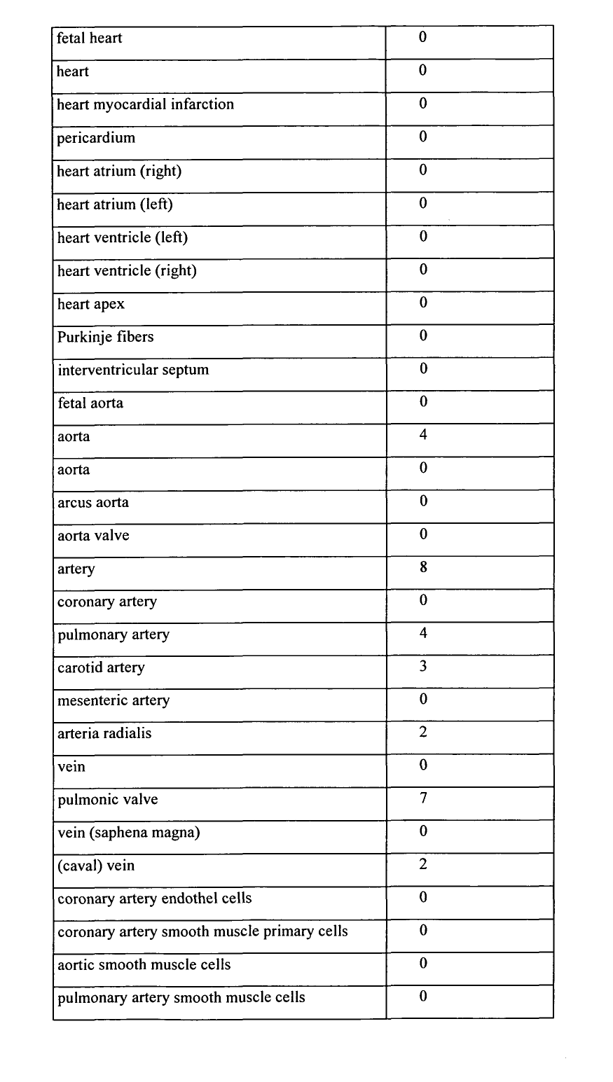

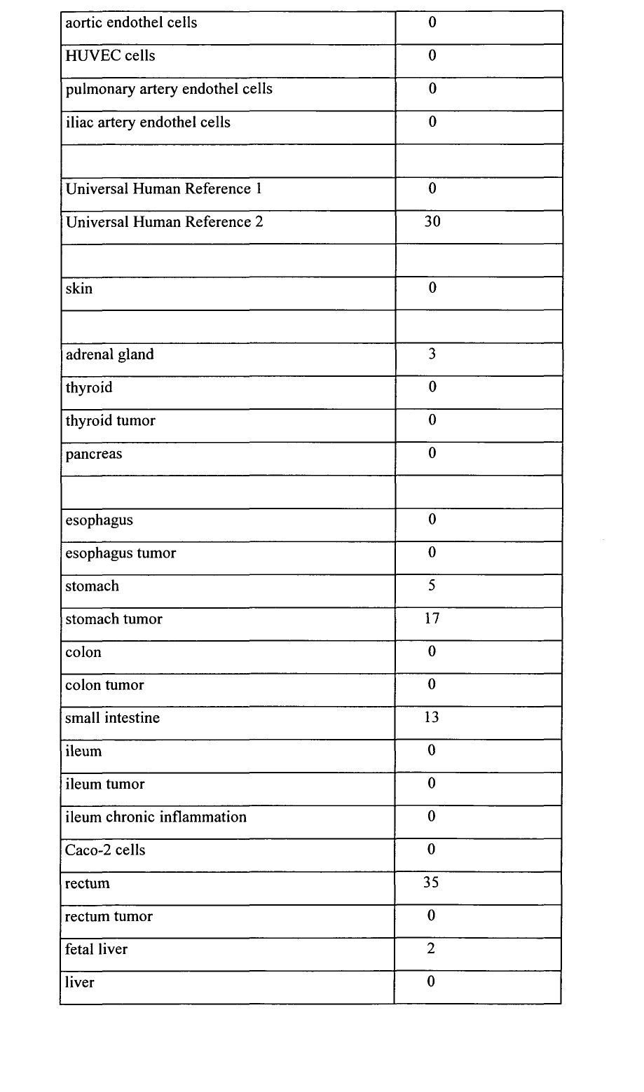

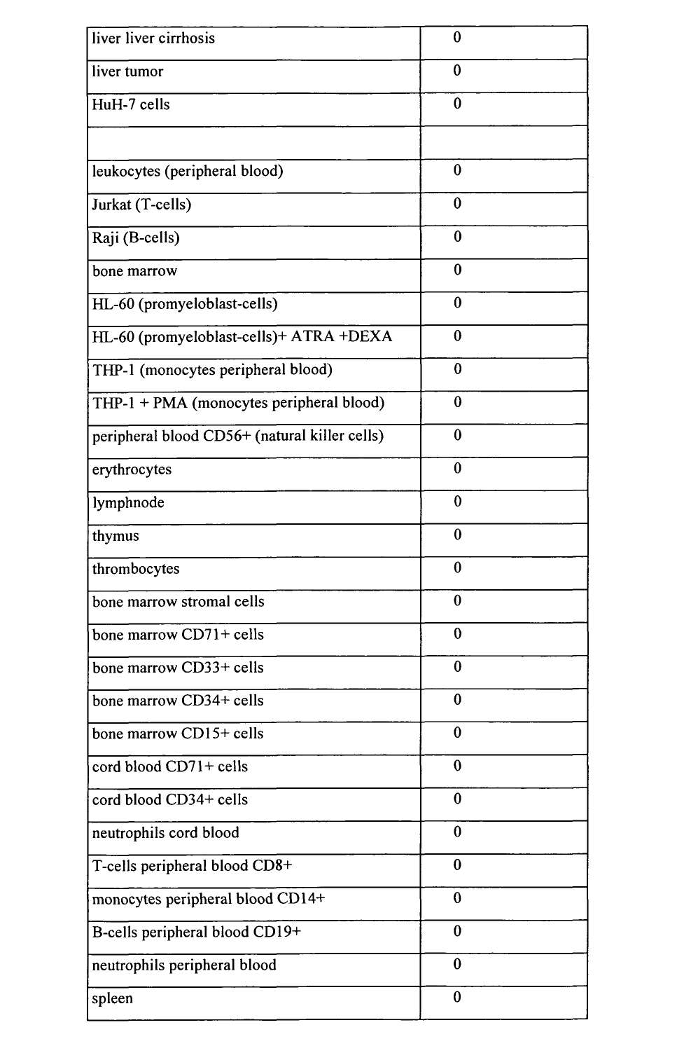

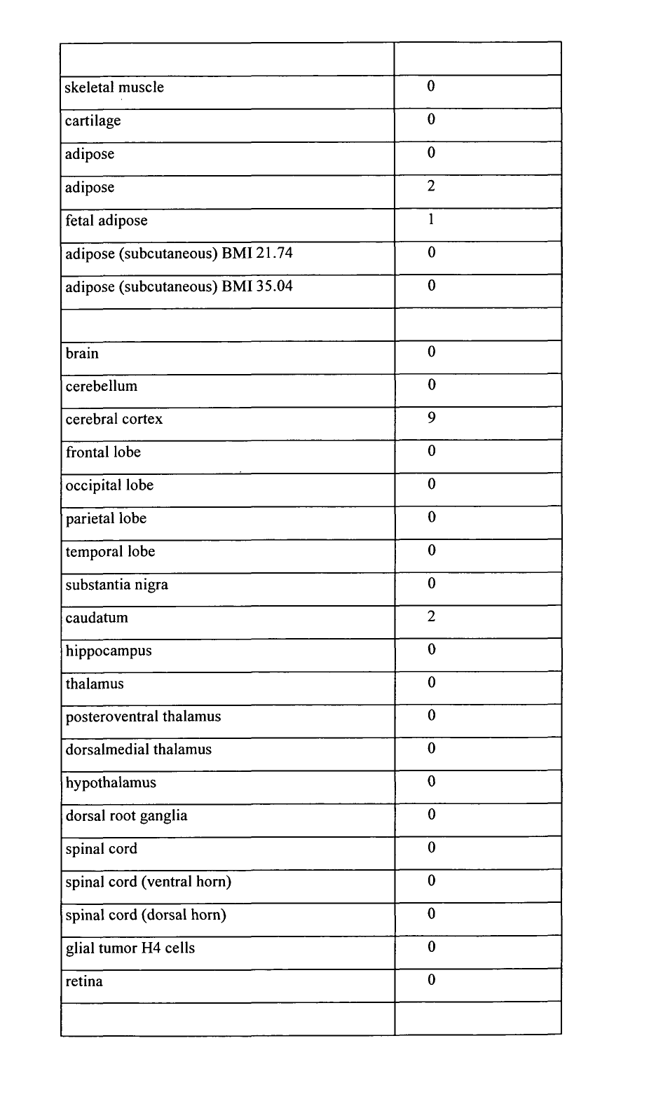

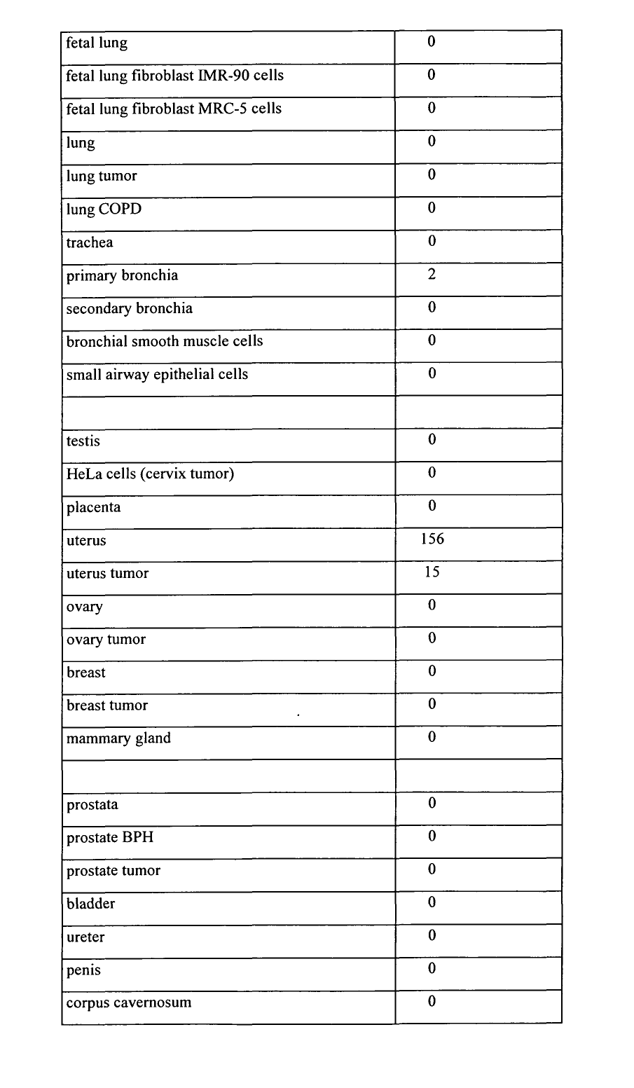

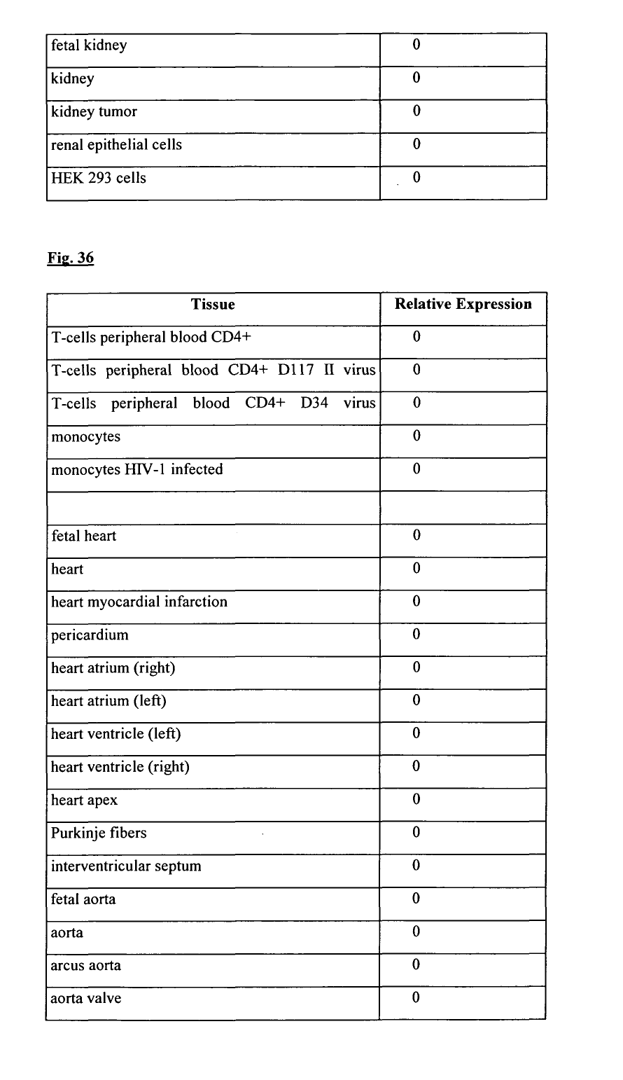

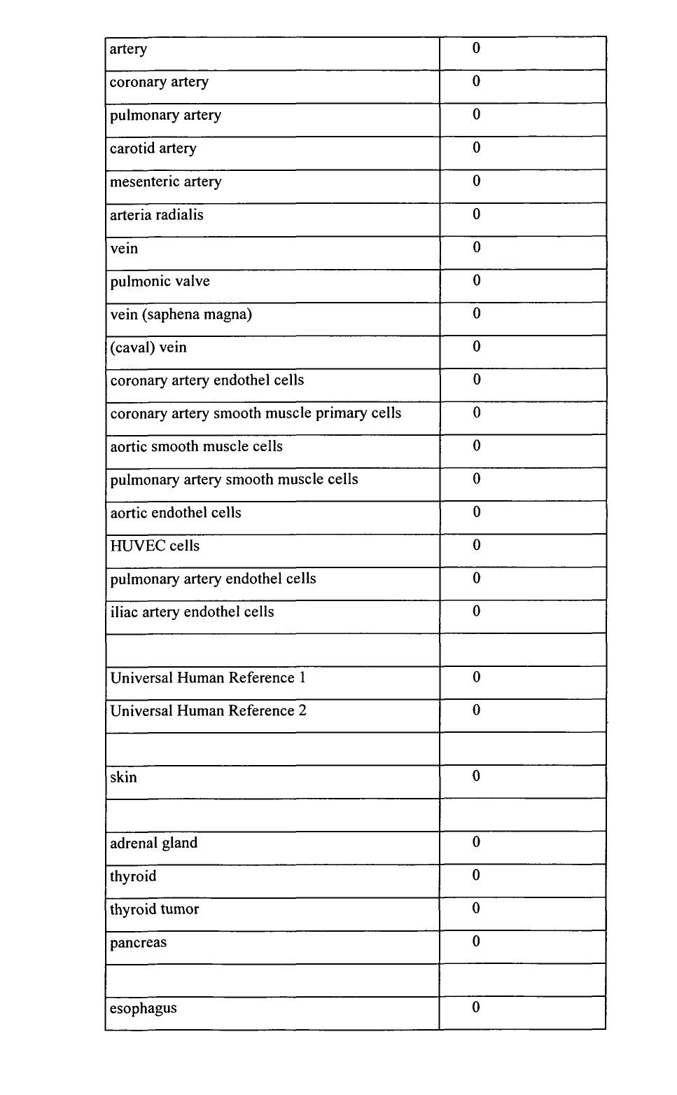

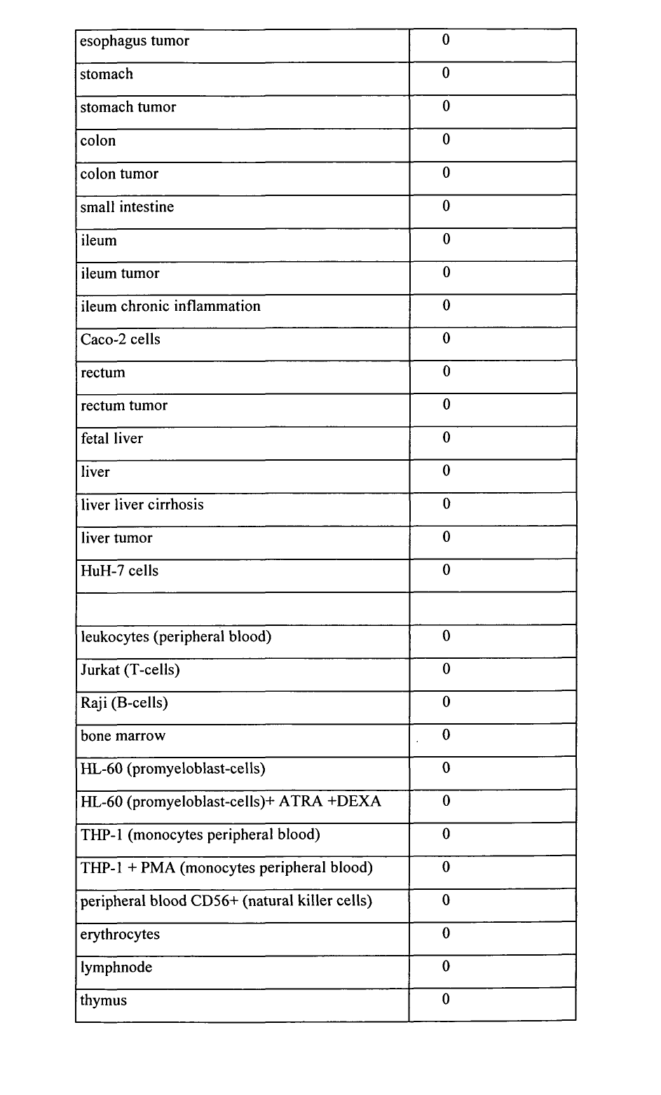

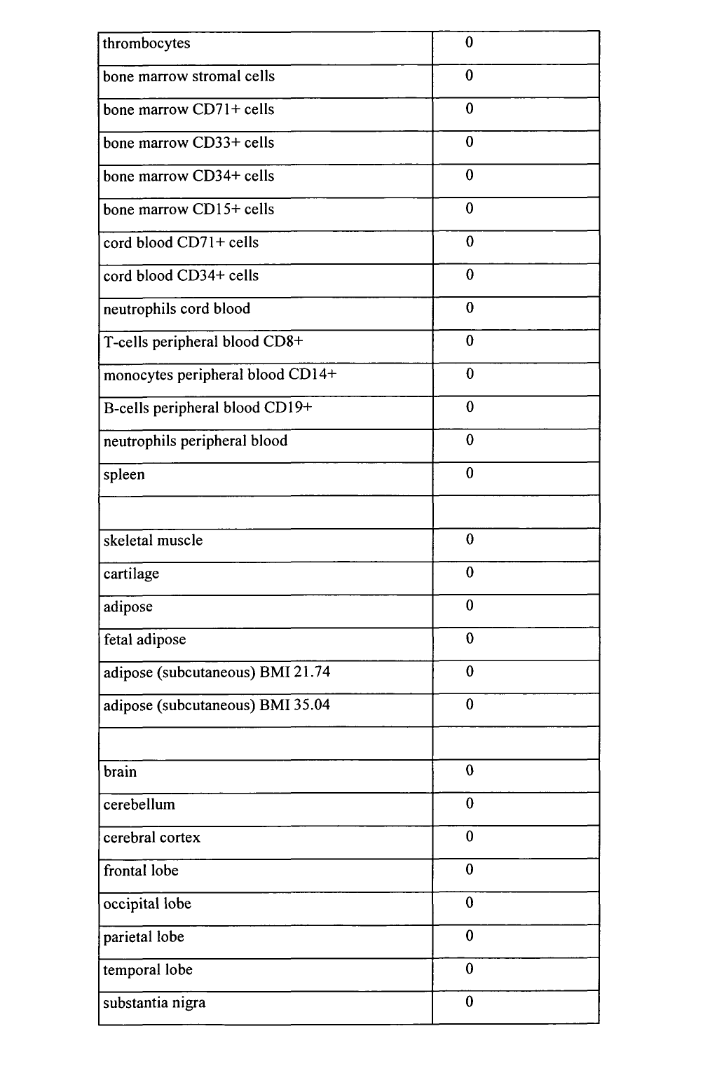

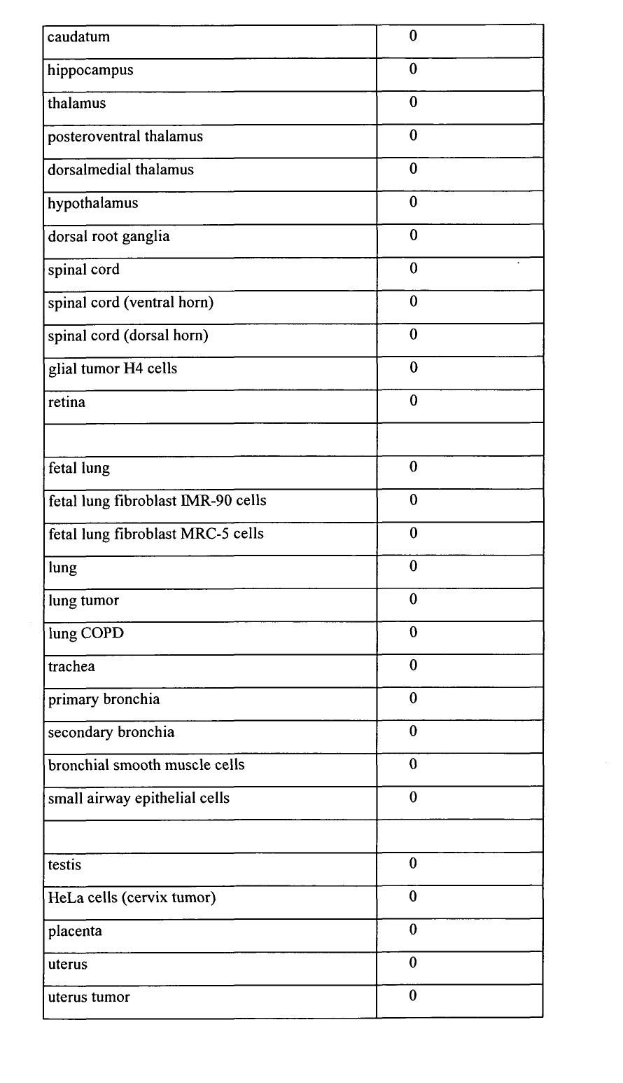

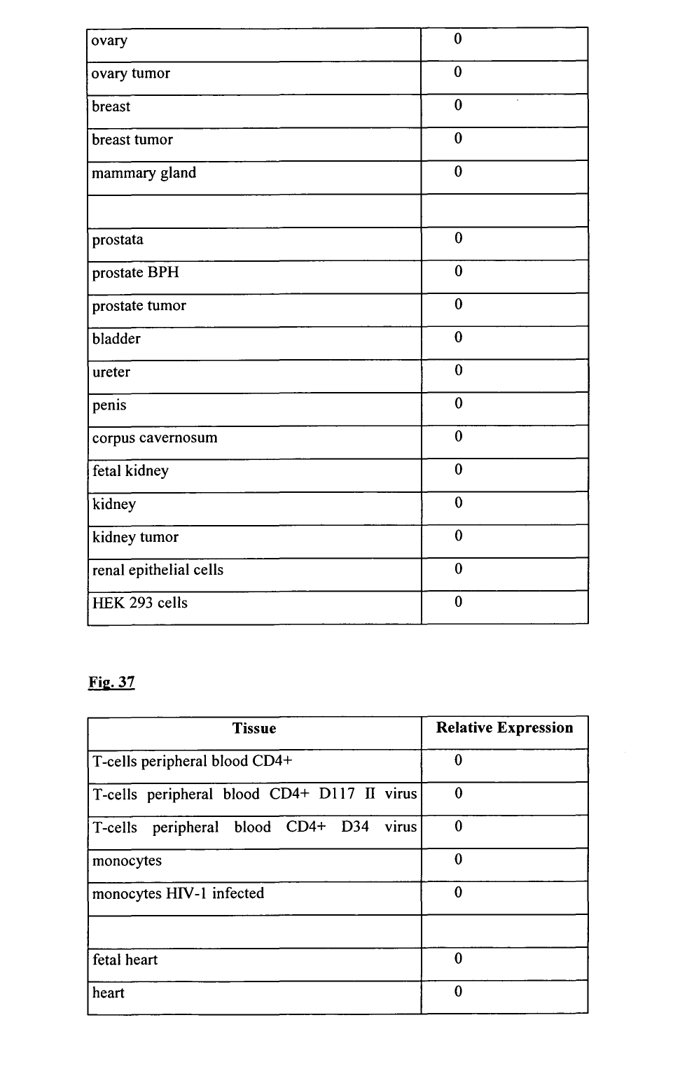

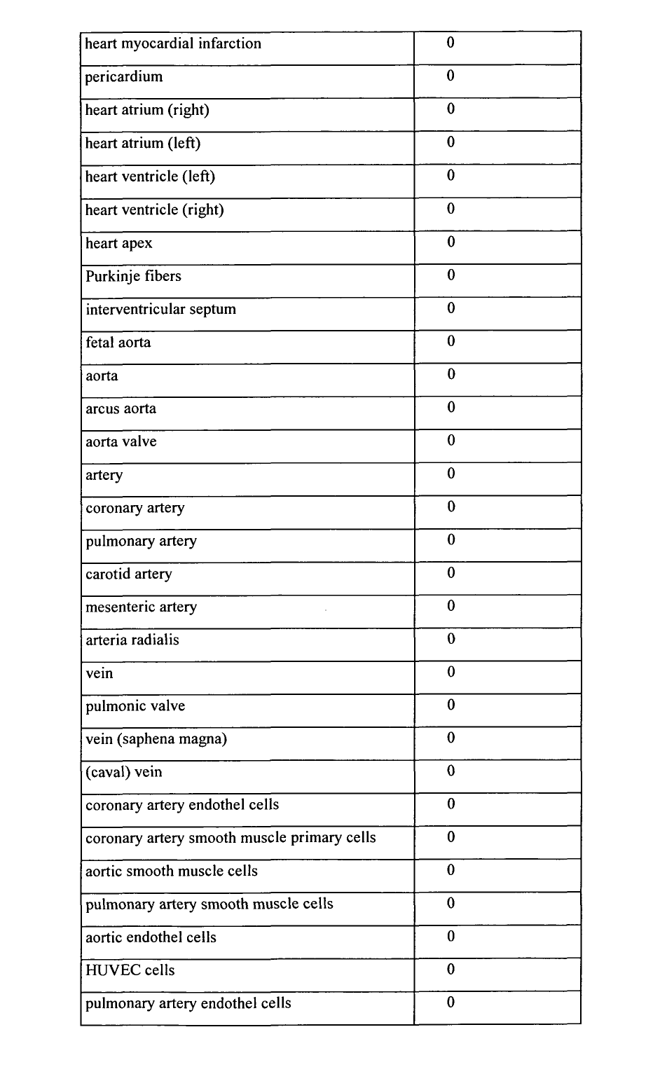

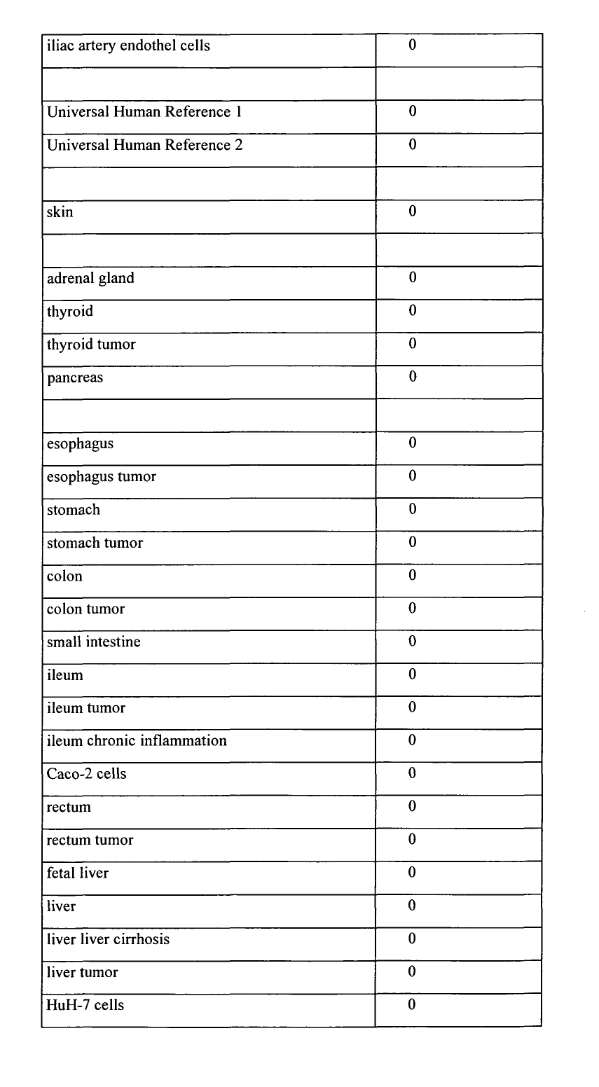

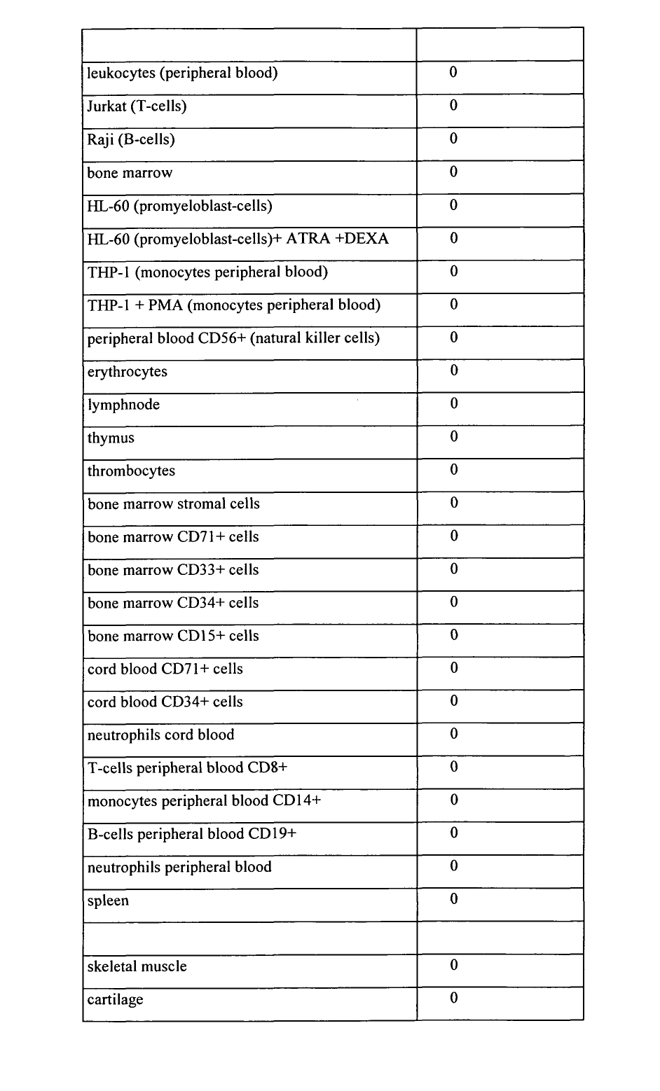

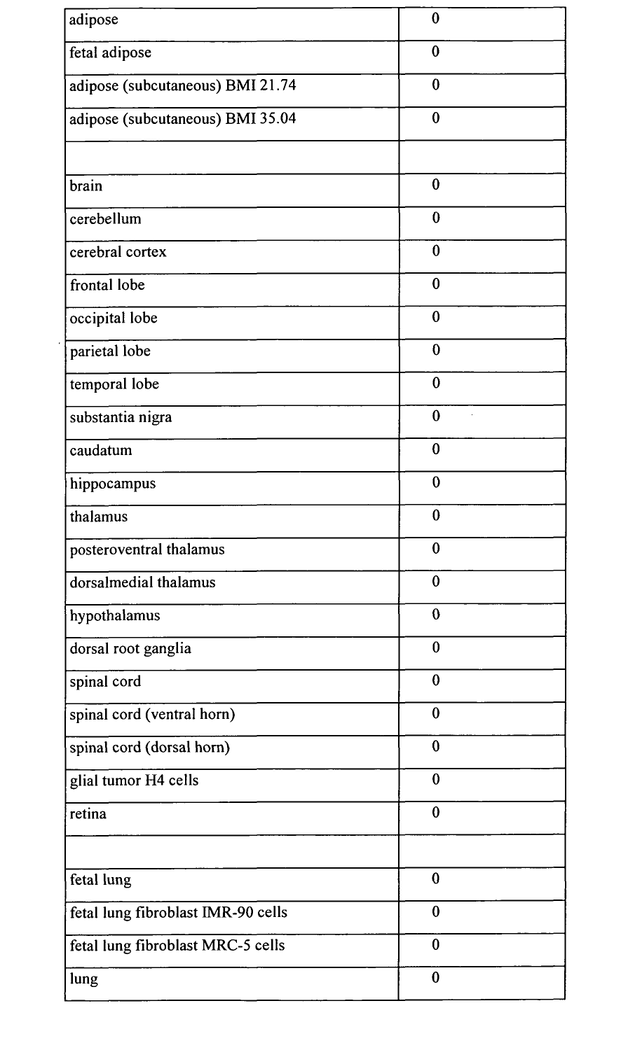

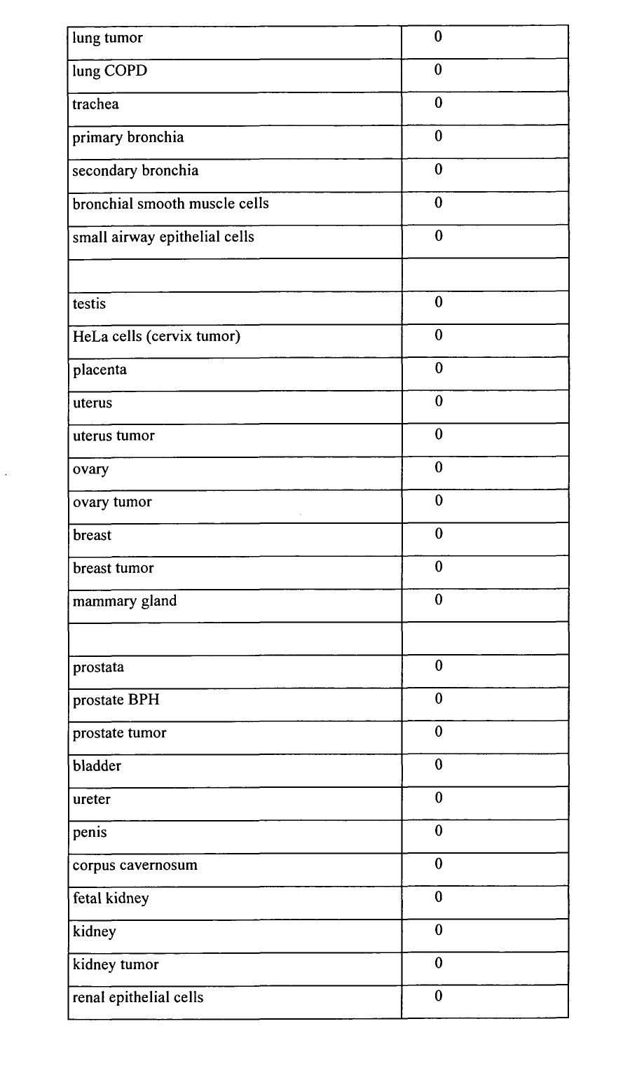

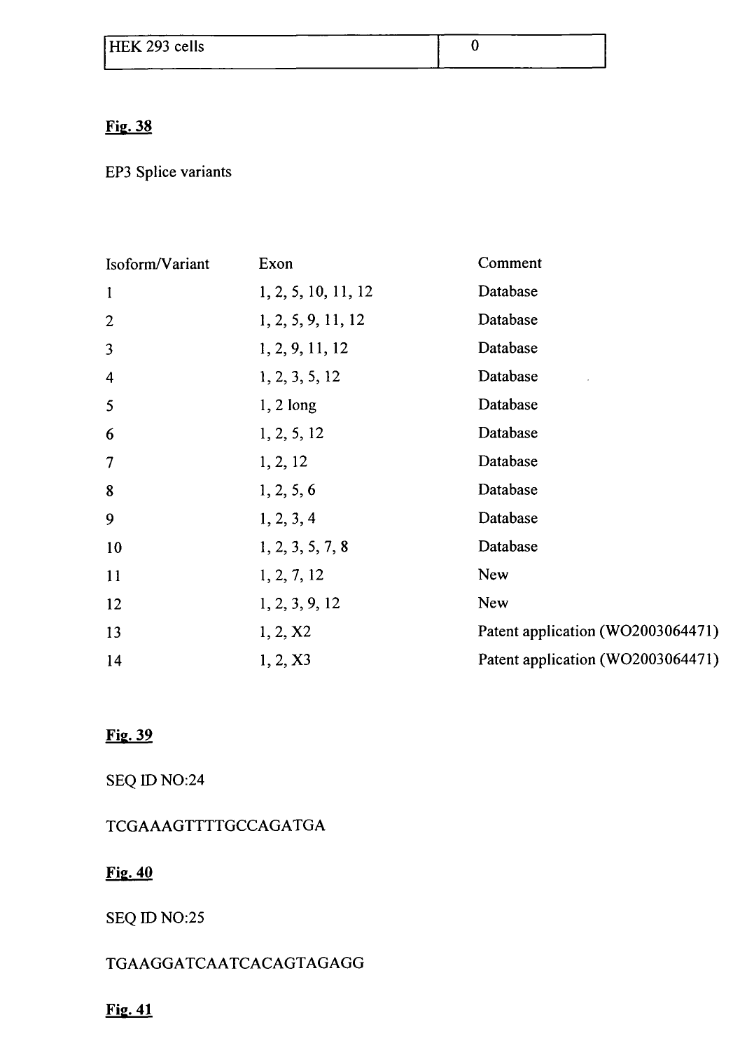

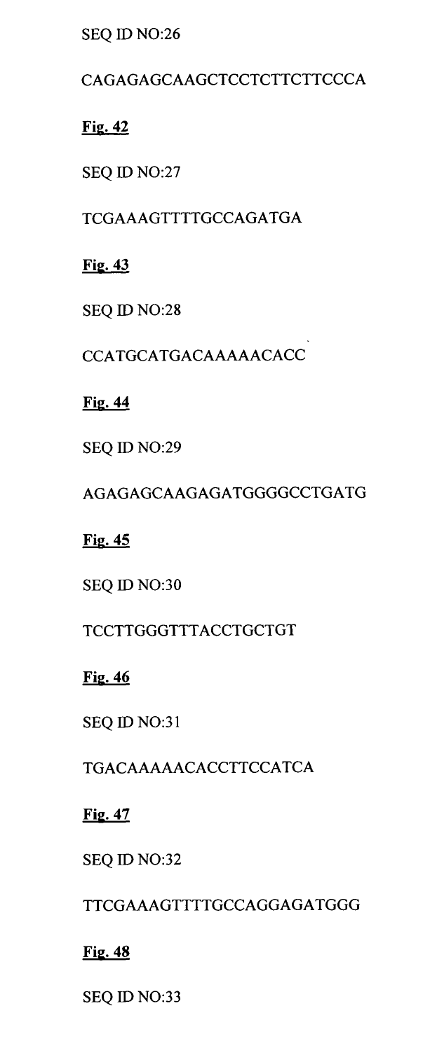

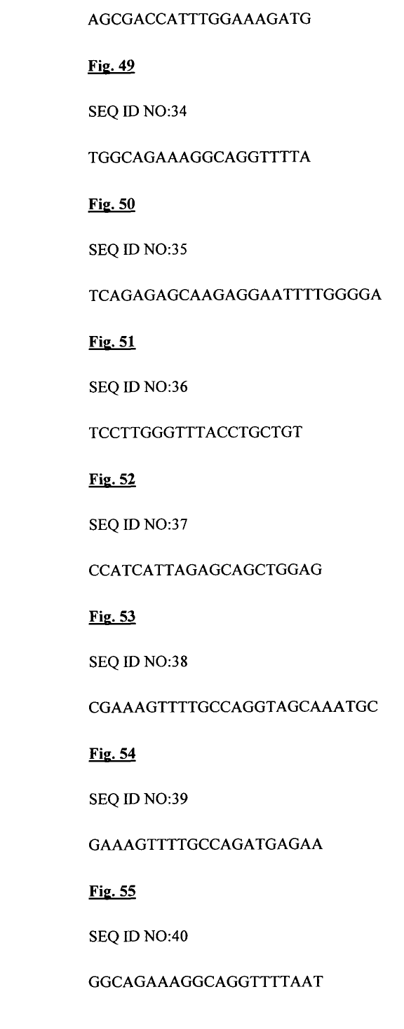

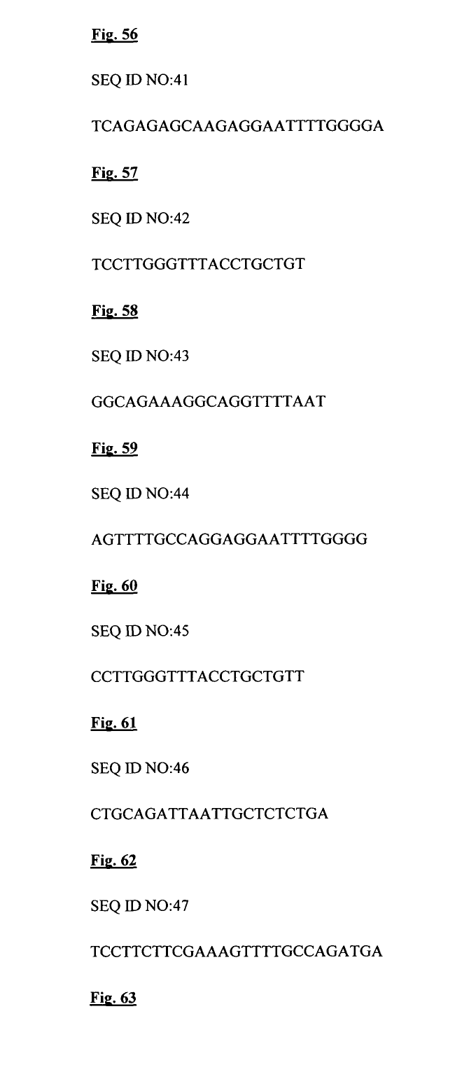

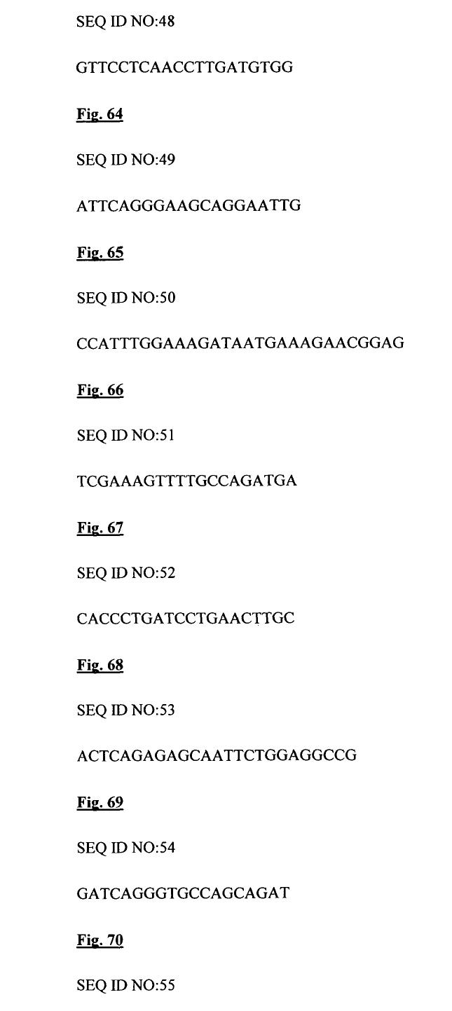

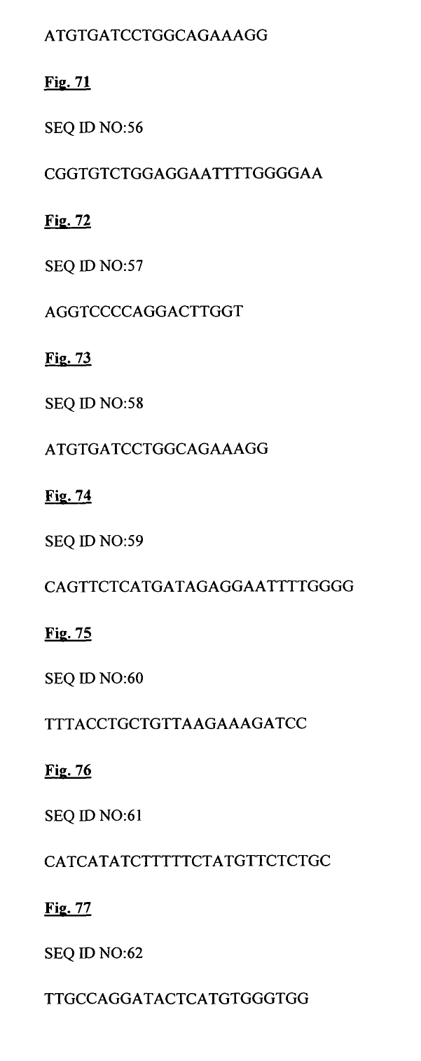

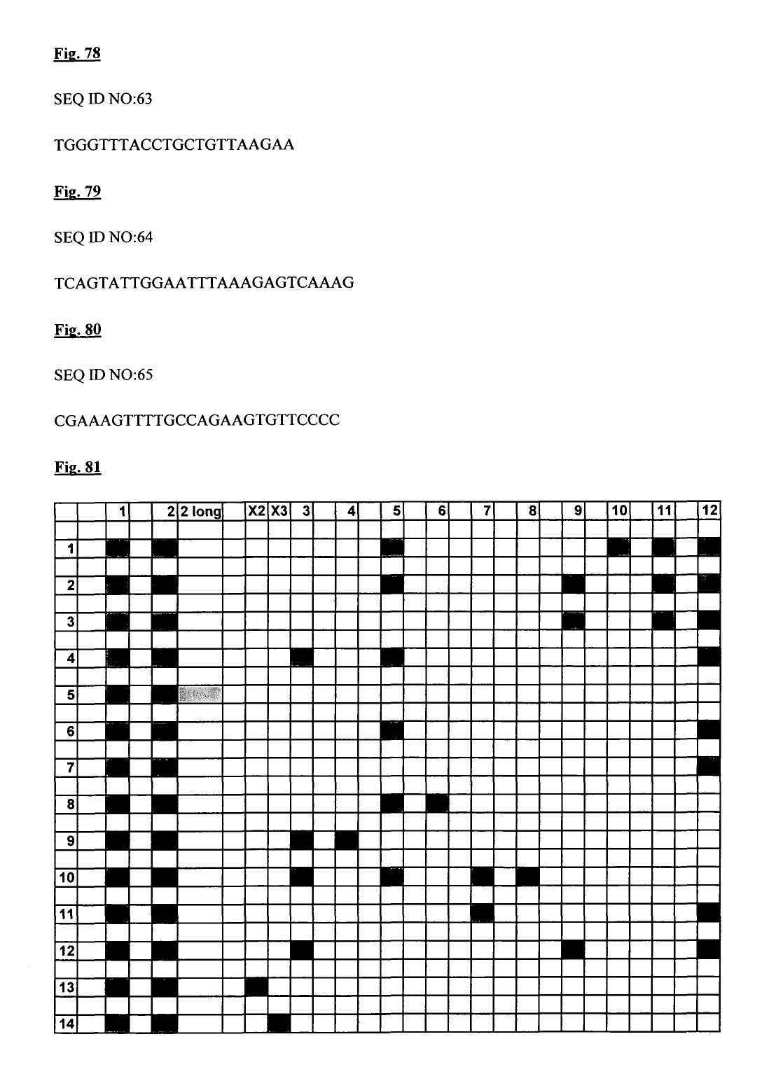

### DETAILED DESCRIPTION OF THE INVENTION

### New Splice Variants or Isoforms

The identified splice variants EP3-11 and EP3-12 were assembled using ESTs BG209275.1 (EP3-11/Exon 7 to 12/SEQ ID No:18) and BG192181.1 (EP-3-12/Exon 9 to 12/SEQ ID No:19) and known sequences from public databases. The expression profiles or EP3-11 and EP3-12 are shown in FIGS. 34 and 35. Surprisingly both splice variants are specifically expressed in human tissues.

As used herein and designated by the upper case abbreviation, EP3-11 or EP3-12, refer to GPCRs in either naturally occurring or synthetic form and active fragments thereof which have the amino acid sequence of SEQ ID NO:22 or SEQ ID NO:23. In one embodiment, the polypeptide EP3-11 or EP3-12 is encoded by mRNAs transcribed from the cDNA, as designated by the lower case abbreviation, EP3-11 or EP3-12, of SEQ ID NO:20 or SEQ ID NO:21.

The nucleotide sequences encoding EP3-11 OR EP3-12 (or their complement) have numerous applications in techniques known to those skilled in the art of molecular biology. These techniques include use as hybridization probes, use in the construction of oligomers for PCR, use for chromosome and gene mapping, use in the recombinant production of EP3-11 OR EP3-12, and use in generation of antisense DNA or RNA, their chemical analogs and the like. Uses of nucleotides encoding EP3-11 OR EP3-12 disclosed herein are exemplary of known techniques and are not intended to limit their use in any technique known to a person of ordinary skill in the art. Furthermore, the nucleotide sequences disclosed herein may be used in molecular biology techniques that have not yet been developed, provided the new techniques rely on properties of nucleotide sequences that are currently known, e.g., the triplet genetic code, specific base pair interactions, etc.

It will be appreciated by those skilled in the art that as a result of the degeneracy of the genetic code, a multitude of EP3-11 OR EP3-12-encoding nucleotide sequences may be produced. Some of these will only bear minimal homology to the nucleotide sequence of the known and naturally occurring EP3-11 OR EP3-12. The invention has specifically contemplated each and every possible variation of nucleotide sequence that could be made by selecting combinations based on possible codon choices. These combinations are made in accordance with the standard triplet genetic code as applied to the nucleotide sequence of naturally occurring EP3-11 OR EP3-12, and all such variations are to be considered as being specifically disclosed.

Although the nucleotide sequences which encode EP3-11 OR EP3-12, its derivatives or its variants are preferably capable of hybridizing to the nucleotide sequence of the naturally occurring EP3-11 OR EP3-12 under stringent conditions, it may be advantageous to produce nucleotide sequences encoding EP3-11 OR EP3-12 or its derivatives possessing a substantially different codon usage. Codons can be selected to increase the rate at which expression of the peptide occurs in a particular prokaryotic or eukaryotic expression host in accordance with the frequency with which particular codons are utilized by the host. Other reasons for substantially altering the nucleotide sequence encoding EP3-11 OR EP3-12 and/or its derivatives without altering the encoded amino acid sequence include the production of RNA transcripts having more desirable properties, such as a greater half-life, than transcripts produced from the naturally occurring sequence.

Nucleotide sequences encoding EP3-11 OR EP3-12 may be joined to a variety of other nucleotide sequences by means of well established recombinant DNA techniques. Useful nucleotide sequences for joining to EP3-11 OR EP3-12 include an assortment of cloning vectors such as plasmids, cosmids, lambda phage derivatives, phagemids, and the like. Vectors of interest include expression vectors, replication vectors, probe generation vectors, sequencing vectors, etc. In general, vectors of interest may contain an origin of replication functional in at least one organism, convenient restriction endonuclease sensitive sites, and selectable markers for one or more host cell systems.

Another aspect of the subject invention is to provide for EP3-11 OR EP3-12-specific hybridization probes capable of hybridizing with naturally occurring nucleotide sequences encoding EP3-11 OR EP3-12. Such probes may also be used for the detection of similar GPCR encoding sequences and should preferably contain at least 40% nucleotide identity to EP3-11 OR EP3-12 sequence. The hybridization probes of the subject invention may be derived from the nucleotide sequence presented as SEQ ID NO:20 or SEQ ID NO:21 or from genomic sequences including promoter, enhancers or introns of the native gene. Hybridization probes may be labeled by a variety of reporter molecules using techniques well known in the art.

It will be recognized that many deletional or mutational analogs of nucleic acid sequences for EP3-11 OR EP3-12 will be effective hybridization probes for EP3-11 OR EP3-12 nucleic acid. Accordingly, the invention relates to nucleic acid sequences that hybridize with such EP3-11 OR EP3-12 encoding nucleic acid sequences under stringent conditions.

“Stringent conditions” refers to conditions that allow for the hybridization of substantially related nucleic acid sequences. For instance, such conditions will generally allow hybridization of sequence with at least about 85% sequence identity, preferably with at least about 90% sequence identity, more preferably with at least about 95% sequence identity. Hybridization conditions and probes can be adjusted in well-characterized ways to achieve selective hybridization of human-derived probes.

Nucleic acid molecules that will hybridize to EP3-11 OR EP3-12 encoding nucleic acid under stringent conditions can be identified functionally. Without limitation, examples of the uses for hybridization probes include: histochemical uses such as identifying tissues that express EP3-11 OR EP3-12; measuring mRNA levels, for instance to identify a sample's tissue type or to identify cells that express abnormal levels of EP3-11 OR EP3-12; and detecting polymorphisms in EP3-11 OR EP3-12.

PCR as described in U.S. Pat. Nos. 4,683,195; 4,800,195; and 4,965,188 provides additional uses for oligonucleotides based upon the nucleotide sequence which encodes EP3-11 OR EP3-12. Such probes used in PCR may be of recombinant origin, chemically synthesized, or a mixture of both. Oligomers may comprise discrete nucleotide sequences employed under optimized conditions for identification of EP3-11 OR EP3-12 in specific tissues or diagnostic use. The same two oligomers, a nested set of oligomers, or even a degenerate pool of oligomers may be employed under less stringent conditions for identification of closely related DNAs or RNAs.

Rules for designing polymerase chain reaction (“PCR”) primers are now established, as reviewed by PCR Protocols. Degenerate primers, i.e., preparations of primers that are heterogeneous at given sequence locations, can be designed to amplify nucleic acid sequences that are highly homologous to, but not identical with EP3-11 OR EP3-12. Strategies are now available that allow for only one of the primers to be required to specifically hybridize with a known sequence. For example, appropriate nucleic acid primers can be ligated to the nucleic acid sought to be amplified to provide the hybridization partner for one of the primers. In this way, only one of the primers need be based on the sequence of the nucleic acid sought to be amplified.

PCR methods for amplifying nucleic acid will utilize at least two primers. One of these primers will be capable of hybridizing to a first strand of the nucleic acid to be amplified and of priming enzyme-driven nucleic acid synthesis in a first direction. The other will be capable of hybridizing the reciprocal sequence of the first strand (if the sequence to be amplified is single stranded, this sequence will initially be hypothetical, but will be synthesized in the first amplification cycle) and of priming nucleic acid synthesis from that strand in the direction opposite the first direction and towards the site of hybridization for the first primer. Conditions for conducting such amplifications, particularly under preferred stringent hybridization conditions, are well known.

Other means of producing specific hybridization probes for EP3-11 OR EP3-12 include the cloning of nucleic acid sequences encoding EP3-11 OR EP3-12 or EP3-11 OR EP3-12 derivatives into vectors for the production of mRNA probes. Such vectors are known in the art, are commercially available and may be used to synthesize RNA probes in vitro by means of the addition of the appropriate RNA polymerase as T7 or SP6 RNA polymerase and the appropriate reporter molecules.

It is possible to produce a DNA sequence, or portions thereof, entirely by synthetic chemistry. After synthesis, the nucleic acid sequence can be inserted into any of the many available DNA vectors and their respective host cells using techniques which are well known in the art. Moreover, synthetic chemistry may be used to introduce mutations into the nucleotide sequence. Alternately, a portion of sequence in which a mutation is desired can be synthesized and recombined with longer portion of an existing genomic or recombinant sequence.

Nucleotide sequences encoding EP3-11 OR EP3-12 may be used to produce a purified oligo-or polypeptide using well known methods of recombinant DNA technology. The oligopeptide may be expressed in a variety of host cells, either prokaryotic or eukaryotic. Host cells may be from the same species from which the nucleotide sequence was derived or from a different species. Advantages of producing an oligonucleotide by recombinant DNA technology include obtaining adequate amounts of the protein for purification and the availability of simplified purification procedures.

### DEFINITIONS

“Animal” as used herein may be defined to include human, domestic (e.g., cats, dogs, etc.), agricultural (e.g., cows, horses, sheep, etc.) or test species (e.g., mouse, rat, rabbit, etc.).

“Biomarker” are measurable and quantifiable biological parameters (e.g. specific enzyme concentration, specific hormone concentration, specific gene phenotype distribution in a population, presence of biological substances) which serve as indices for health—and physiology related assessments, such as disease risk, psychiatric disorders, environmental exposure and its effects, disease diagnosis, metabolic processes, substance abuse, pregnancy, cell line development, epidemiologic studies, etc. Parameter that can be used to identify a toxic effect in an individual organism and can be used in extrapolation between species. Indicator signalling an event or condition in a biological system or sample and giving a measure of exposure, effect, or susceptibility.

Biological markers can reflect a variety of disease characteristics, including the level of exposure to an environmental or genetic trigger, an element of the disease process itself, an intermediate stage between exposure and disease onset, or an independent factor associated with the disease state but not causative of pathogenesis. Depending on the specific characteristic, biomarkers can be used to identify the risk of developing an illness (antecedent biomarkers), aid in identifying disease (diagnostic biomarkers), or predict future disease course, including response to therapy (prognostic biomarkers).

An “oligonucleotide” is a stretch of nucleotide residues which has a sufficient number of bases to be used as an oligomer, amplimer or probe in a polymerase chain reaction (PCR). Oligonucleotides are prepared from genomic or cDNA sequence and are used to amplify, reveal, or confirm the presence of a similar DNA or RNA in a particular cell or tissue. Oligonucleotides or oligomers comprise portions of a DNA sequence having at least about 10 nucleotides and as many as about 35 nucleotides, preferably about 25 nucleotides.

“Probes” may be derived from naturally occurring or recombinant single- or double-stranded nucleic acids or may be chemically synthesized. They are useful in detecting the presence of identical or similar sequences. Such probes may be labeled with reporter molecules using nick translation, Klenow fill-in reaction, PCR or other methods well known in the art. Nucleic acid probes may be used in southern, northern or in situ hybridizations to determine whether DNA or RNA encoding a certain protein is present in a cell type, tissue, or organ.

A fragment of a polynucleotide or nucleic acid that comprises all or any part of the nucleotide sequence having fewer nucleotides than about 6 kb, preferably fewer than about 1 kb which can be used as a probe.

“Reporter” molecules are radionuclides, enzymes, fluorescent, chemiluminescent, or chromogenic agents which associate with a particular nucleotide or amino acid sequence, thereby establishing the presence of a certain sequence, or allowing for the quantification of a certain sequence.

“Recombinant nucleotide variants” encoding EP3-11 or EP3-12 may be synthesized by making use of the “redundancy” in the genetic code. Various codon substitutions, such as the silent changes which produce specific restriction sites or codon usage-specific mutations, may be introduced to optimize cloning into a plasmid or viral vector or expression in a particular prokaryotic or eukaryotic host system, respectively.

“Chimeric” molecules may be constructed by introducing all or part of the nucleotide sequence of this invention into a vector containing additional nucleic acid sequence which might be expected to change any one or several of the following EP3-11 OR EP3-12 characteristics: cellular location, distribution, ligand-binding affinities, interchain affinities, degradation/turnover rate, signaling, etc.

“Active” refers to those forms, fragments, or domains of EP3-11 OR EP3-12 which retain the biological and/or antigenic activities of EP3-11 OR EP3-12.

“Naturally occurring EP3-11 OR EP3-12” refers to a polypeptide produced by cells which have not been genetically engineered and specifically contemplates various polypeptides arising from post-translational modifications of the polypeptide including but not limited to acetylation, carboxylation, glycosylation, phosphorylation, lipidation and acylation.

“Derivative” refers to polypeptides which have been chemically modified by techniques such as ubiquitination, labeling (see above), pegylation (derivatization with polyethylene glycol), and chemical insertion or substitution of amino acids such as ornithine which do not normally occur in human proteins.

“Recombinant polypeptide variant” refers to any polypeptide which differs from naturally occurring EP3-11 OR EP3-12 by amino acid insertions, deletions and/or substitutions, created using recombinant DNA techniques. Guidance in determining which amino acid residues may be replaced, added, or deleted, without abolishing activities of interest may be found by comparing the sequence of the polypeptide of interest with that of related polypeptides and minimizing the number of amino acid sequence changes made in highly conserved regions.

Conservative Amino acid “substitutions” result from replacing one amino acid with another having similar structural and/or chemical properties, such as the replacement of a leucine with an isoleucine or valine, an aspartate with a glutamate, or a threonine with a serine.

“Insertions” or “deletions” are typically in the range of about 1 to 5 amino acids. The variation allowed may be experimentally determined by producing the peptide synthetically while systematically making insertions, deletions, or substitutions of nucleotides in the sequence using recombinant DNA techniques.

A “signal or leader sequence” can be used, when desired, to direct the polypeptide through a membrane of a cell. Such a sequence may be naturally present on the polypeptides of the present invention or provided from heterologous sources by recombinant DNA techniques.

An “oligopeptide” is a short stretch of amino acid residues and may be expressed from an oligonucleotide. Oligopeptides comprise a stretch of amino acid residues of at least 3, 5, 10 amino acids and at most 10, 15, 25 amino acids, typically of at least 9 to 13 amino acids, and of sufficient length to display biological and/or antigenic activity.

“Inhibitor” is any substance which retards or prevents a chemical or physiological reaction or response. Common inhibitors include but are not limited to antisense molecules, antibodies, and antagonists.

“Standard” expression is a quantitative or qualitative measurement for comparison. It is based on a statistically appropriate number of normal samples and is created to use as a basis of comparison when performing diagnostic assays, running clinical trials, or following patient treatment profiles.

“Animal” as used herein may be defined to include human, domestic (cats dogs, etc.), agricultural (cows, horses, sheep, etc.) or test species (mouse, rat, rabbit, etc.).

### Quantitative Determinations of Nucleic Acids

An important step in the molecular genetic analysis of human disease is often the enumeration of the copy number of a nucleic acid or the relative expression of a gene in particular tissues.

Several different approaches are currently available to make quantitative determinations of nucleic acids. Chromosome-based techniques, such as comparative genomic hybridization (CGH) and fluorescent in situ hybridization (FISH) facilitate efforts to cytogenetically localize genomic regions that are altered in tumor cells. Regions of genomic alteration can be narrowed further using loss of heterozygosity analysis (LOH), in which disease DNA is analyzed and compared with normal DNA for the loss of a heterozygous polymorphic marker. The first experiments used restriction fragment length polymorphisms (RFLPs) [Johnson et al], or hypervariable minisatellite DNA [Barnes et al, 2000]. In recent years LOH has been performed primarily using PCR amplification of microsatellite markers and electrophoresis of the radiolabeled [Jeffreys et al, 1985] or fluorescently labeled PCR products [Weber et al, 1990] and compared between paired normal and disease DNAs.

A number of other methods have also been developed to quantify nucleic acids. More recently, PCR and RT-PCR methods have been developed which are capable of measuring the amount of a nucleic acid in a sample.

A gene sequence contained in all samples at relatively constant quantity is typically utilized for sample amplification efficiency normalization. This approach, however, suffers from several drawbacks. The method requires that each sample has equal input amounts of the nucleic acid and that the amplification efficiency between samples is identical until the time of analysis. Furthermore, it is difficult using the conventional methods of PCR quantitation such as gel electrophoresis or plate capture hybridization to determine that all samples are in fact analyzed during the log phase of the reaction as required by the method.

Another method called quantitative competitive (QC)-PCR, as the name implies, relies on the inclusion of an internal control competitor in each reaction. The efficiency of each reaction is normalized to the internal competitor. A known amount of internal competitor is typically added to each sample. The unknown target PCR product is compared with the known competitor PCR product to obtain relative quantitation. A difficulty with this general approach lies in developing an internal control that amplifies with the same efficiency than the target molecule.

### 5′ Fluorogenic Nuclease Assays

Fluorogenic nuclease assays are a real time quantitation method that uses a probe to monitor formation of amplification product. The basis for this method of monitoring the formation of amplification product is to measure continuously PCR product accumulation using a dual-labeled fluorogenic oligonucleotide probe, an approach frequently referred to in the literature simply as the “TaqMan method” [Piatak et al., 1993, Heid et al. 1995, Gibson et al, 1996, Holland et al., 1991].

The probe used in such assays is typically a short (about 20-25 bases) oligonucleotide that is labeled with two different fluorescent dyes. The 5′ terminus of the probe is attached to a reporter dye and the 3′ terminus is attached to a quenching dye, although the dyes could be attached at other locations on the probe as well. The probe is designed to have at least substantial sequence complementarity with the probe binding site. Upstream and downstream PCR primers which bind to flanking regions of the locus are added to the reaction mixture. When the probe is intact, energy transfer between the two fluorophors occurs and the quencher quenches emission from the reporter. During the extension phase of PCR, the probe is cleaved by the 5′ nuclease activity of a nucleic acid polymerase such as Taq polymerase, thereby releasing the reporter from the oligonucleotide-quencher and resulting in an increase of reporter emission intensity which can be measured by an appropriate detector.

One detector which is specifically adapted for measuring fluorescence emissions such as those created during a fluorogenic assay is the ABI 7700 or 4700 HT manufactured by Applied Biosystems, Inc. in Foster City, Calif. The ABI 7700 uses fiber optics connected with each well in a 96-or 384 well PCR tube arrangement. The instrument includes a laser for exciting the labels and is capable of measuring the fluorescence spectra intensity from each tube with continuous monitoring during PCR amplification. Each tube is reexamined every 8.5 seconds.

Computer software provided with the instrument is capable of recording the fluorescence intensity of reporter and quencher over the course of the amplification. The recorded values will then be used to calculate the increase in normalized reporter emission intensity on a continuous basis. The increase in emission intensity is plotted versus time, i.e., the number of amplification cycles, to produce a continuous measure of amplification. To quantify the locus in each amplification reaction, the amplification plot is examined at a point during the log phase of product accumulation. This is accomplished by assigning a fluorescence threshold intensity above background and determining the point at which each amplification plot crosses the threshold (defined as the threshold cycle number or Ct). Differences in threshold cycle number are used to quantify the relative amount of PCR target contained within each tube. Assuming that each reaction functions at 100% PCR efficiency, a difference of one Ct represents a two-fold difference in the amount of starting template. The fluorescence value can be used in conjunction with a standard curve to determine the amount of amplification product present.

### Non-Probe-Based Detection Methods

A variety of options are available for measuring the amplification products as they are formed. One method utilizes labels, such as dyes, which only bind to double stranded DNA. In this type of approach, amplification product (which is double stranded) binds dye molecules in solution to form a complex. With the appropriate dyes, it is possible to distinguish between dye molecules free in solution and dye molecules bound to amplification product. For example, certain dyes fluoresce only when bound to amplification product. Examples of dyes which can be used in methods of this general type include, but are not limited to, Syber Green™ and Pico Green from Molecular Probes, Inc. of Eugene, Oreg., ethidium bromide, propidium iodide, chromomycin, acridine orange, Hoechst 33258, Toto-1, Yoyo-1, DAPI (4′,6-diamidino-2-phenylindole hydrochloride).

### Probe-Based Detection Methods

These detection methods involve some alteration to the structure or conformation of a probe hybridized to the locus between the amplification primer pair. In some instances, the alteration is caused by the template-dependent extension catalyzed by a nucleic acid polymerase during the amplification process. The alteration generates a detectable signal which is an indirect measure of the amount of amplification product formed.

For example, some methods involve the degradation or digestion of the probe during the extension reaction. These methods are a consequence of the 5′-3′ nuclease activity associated with some nucleic acid polymerases. Polymerases having this activity cleave mononucleotides or small oligonucleotides from an oligonucleotide probe annealed to its complementary sequence located within the locus.

The 3′ end of the upstream primer provides the initial binding site for the nucleic acid polymerase. As the polymerase catalyzes extension of the upstream primer and encounters the bound probe, the nucleic acid polymerase displaces a portion of the 5′ end of the probe and through its nuclease activity cleaves mononucleotides or oligonucleotides from the probe.

The upstream primer and the probe can be designed such that they anneal to the complementary strand in close proximity to one another. In fact, the 3′ end of the upstream primer and the 5′ end of the probe may abut one another. In this situation, extension of the upstream primer is not necessary in order for the nucleic acid polymerase to begin cleaving the probe. In the case in which intervening nucleotides separate the upstream primer and the probe, extension of the primer is necessary before the nucleic acid polymerase encounters the 5′ end of the probe. Once contact occurs and polymerization continues, the 5′-3′ exonuclease activity of the nucleic acid polymerase begins cleaving mononucleotides or oligonucleotides from the 5′ end of the probe. Digestion of the probe continues until the remaining portion of the probe dissociates from the complementary strand.

In solution, the two end sections can hybridize with each other to form a hairpin loop. In this conformation, the reporter and quencher dye are in sufficiently close proximity that fluorescence from the reporter dye is effectively quenched by the quencher dye. Hybridized probe, in contrast, results in a linearized conformation in which the extent of quenching is decreased. Thus, by monitoring emission changes for the two dyes, it is possible to indirectly monitor the formation of amplification product.

### Probes

The labeled probe is selected so that its sequence is substantially complementary to a segment of the test locus or a reference locus. As indicated above, the nucleic acid site to which the probe binds should be located between the primer binding sites for the upstream and downstream amplification primers.

### Primers

The primers used in the amplification are selected so as to be capable of hybridizing to sequences at flanking regions of the locus being amplified. The primers are chosen to have at least substantial complementarity with the different strands of the nucleic acid being amplified. When a probe is utilized to detect the formation of amplification products, the primers are selected in such that they flank the probe, i.e. are located upstream and downstream of the probe.

The primer must have sufficient length so that it is capable of priming the synthesis of extension products in the presence of an agent for polymerization. The length and composition of the primer depends on many parameters, including, for example, the temperature at which the annealing reaction is conducted, proximity of the probe binding site to that of the primer, relative concentrations of the primer and probe and the particular nucleic acid composition of the probe. Typically the primer includes 15-30 nucleotides. However, the length of the primer may be more or less depending on the complexity of the primer binding site and the factors listed above.

### Labels for Probes and Primers

The labels used for labeling the probes or primers of the current invention and which can provide the signal corresponding to the quantity of amplification product can take a variety of forms. As indicated above with regard to the 5′ fluorogenic nuclease method, a fluorescent signal is one signal which can be measured. However, measurements may also be made, for example, by monitoring radioactivity, colorimetry, absorption, magnetic parameters, or enzymatic activity. Thus, labels which can be employed include, but are not limited to, fluorophors, chromophores, radioactive isotopes, electron dense reagents, enzymes, and ligands having specific binding partners (e.g., biotin-avidin).

Monitoring changes in fluorescence is a particularly useful way to monitor the accumulation of amplification products. A number of labels useful for attachment to probes or primers are commercially available including fluorescein and various fluorescein derivatives such as FAM, HEX, TET and JOE (all which are available from Applied Biosystems, Foster City, Calif.); lucifer yellow, and coumarin derivatives.

Labels may be attached to the probe or primer using a variety of techniques and can be attached at the 5′ end, and/or the 3′ end and/or at an internal nucleotide. The label can also be attached to spacer arms of various sizes which are attached to the probe or primer. These spacer arms are useful for obtaining a desired distance between multiple labels attached to the probe or primer.

In some instances, a single label may be utilized; whereas, in other instances, such as with the 5′ fluorogenic nuclease assays for example, two or more labels are attached to the probe. In cases wherein the probe includes multiple labels, it is generally advisable to maintain spacing between the labels which is sufficient to permit separation of the labels during digestion of the probe through the 5′-3′ nuclease activity of the nucleic acid polymerase.

### Patients Exhibiting Symptoms of Disease

A number of diseases are associated with changes in the copy number of a certain gene. For patients having symptoms of a disease, the real-time PCR method can be used to determine if the patient has copy number alterations which are known to be linked with diseases that are associated with the symptoms the patient has.

### EP3-11/EP3-12 Expression

### EP3-11 OR EP3-12 Fusion Proteins

Fusion proteins are useful for generating antibodies against EP3-11 OR EP3-12 amino acid sequences and for use in various assay systems. For example, fusion proteins can be used to identify proteins which interact with portions of EP3-11 OR EP3-12 peptide. Protein affinity chromatography or library-based assays for protein-protein interactions, such as the yeast two-hybrid or phage display systems, can be used for this purpose. Such methods are well known in the art and also can be used as drug screens.

A EP3-11 OR EP3-12 fusion protein comprises two polypeptide segments fused together by means of a peptide bond. The first polypeptide segment can comprise at least 54, 75, 100, 125, 139, 150, 175, 200, 225, 250, or 275 contiguous amino acids of SEQ ID NO:22 SEQ ID NO:23 or of a biologically active variant, such as those described above. The first polypeptide segment also can comprise full-length EP3-11 OR EP3-12.

The second polypeptide segment can be a full-length protein or a protein fragment. Proteins commonly used in fusion protein construction include, but are not limited to β galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, herpes simplex virus (HSV) BP16 protein fusions and G-protein fusions (for example G(alpha)16, Gs, Gi). A fusion protein also can be engineered to contain a cleavage site located adjacent to the EP3-11 OR EP3-12.

### Preparation of Polynucleotides

A naturally occurring EP3-11 OR EP3-12 polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated EP3-11 OR EP3-12 polynucleotides. For example, restriction enzymes and probes can be used to isolate polynucleotide fragments which comprises EP3-11 OR EP3-12 nucleotide sequences. Isolated polynucleotides are in preparations which are free or at least 70, 80, or 90% free of other molecules.

EP3-11 OR EP3-12 cDNA molecules can be made with standard molecular biology techniques, using EP3-11 OR EP3-12 mRNA as a template. EP3-11 OR EP3-12 cDNA molecules can thereafter be replicated using molecular biology techniques known in the art. An amplification technique, such as PCR, can be used to obtain additional copies of polynucleotides of the invention, using either human genomic DNA or cDNA as a template.

Alternatively, synthetic chemistry techniques can be used to synthesizes EP3-11 OR EP3-12 polynucleotides. The degeneracy of the genetic code allows alternate nucleotide sequences to be synthesized which will encode EP3-11 OR EP3-12 having, for example, an amino acid sequence shown in SEQ ID NO:22 or SEQ ID NO:23 or a biologically active variant thereof.

### Extending Polynucleotides

Various PCR-based methods can be used to extend nucleic acid sequences encoding human EP3-11 OR EP3-12, for example to detect upstream sequences of the EP3-11 OR EP3-12 gene such as promoters and regulatory elements. For example, restriction-site PCR uses universal primers to retrieve unknown sequence adjacent to a known locus. Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region. Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA. In this method, multiple restriction enzyme digestions and ligations also can be used to place an engineered double-stranded sequence into an unknown fragment of the DNA molecule before performing PCR.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate equipment and software (e.g. GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA which might be present in limited amounts in a particular sample.

### Obtaining Polypeptides

EP3-11 OR EP3-12 can be obtained, for example, by purification from human cells, by expression of EP3-11 OR EP3-12 polynucleotides, or by direct chemical synthesis.

### Protein Purification

EP3-11 OR EP3-12 can be purified from any human cell which expresses the receptor, including those which have been transfected with expression constructs which express EP3-11 OR EP3-12. A purified EP3-11 OR EP3-12 is separated from other compounds which normally associate with EP3-11 OR EP3-12 in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis.

### Expression of EP3-11 OR EP3-12 Polynucleotides

To express EP3-11 OR EP3-12, EP3-11 OR EP3-12 polynucleotide can be inserted into an expression vector which contains the necessary elements for the transcription and translation of the inserted coding sequence. Methods which are well known to those skilled in the art can be used to construct expression vectors containing sequences encoding EP3-11 OR EP3-12 and appropriate transcriptional and translational control elements. These methods include in vitro recombinant DNA techniques, synthetic techniques, and in vivo genetic recombination.

A variety of expression vector/host systems can be utilized to contain and express sequences encoding EP3-11 OR EP3-12. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding EP3-11 OR EP3-12, vectors based on SV40 or EBV can be used with an appropriate selectable marker.

### Bacterial and Yeast Expression Systems

In bacterial systems, a number of expression vectors can be selected. For example, when a large quantity of EP3-11 OR EP3-12 is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding EP3-11 OR EP3-12 can be ligated into the vector in frame with sequences for the amino-terminal Met and the subsequent 7 residues of β-galactosidase so that a hybrid protein is produced. pIN vectors or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

### Plant and Insect Expression Systems

If plant expression vectors are used, the expression of sequences encoding EP3-11 OR EP3-12 can be driven by any of a number of promoters. For example, viral promoters such as the 35S and 19S promoters of CaMV can be used alone or in combination with the omega leader sequence from TMV. Alternatively, plant promoters such as the small subunit of RUBISCO or heat shock promoters can be used. These constructs can be introduced into plant cells by direct DNA transformation or by pathogen-mediated transfection.

An insect system also can be used to express EP3-11 OR EP3-12. For example, in one such system Autographa califormica nuclear polyhedrosis virus (AcNPV) is used as a vector to express foreign genes in Spodoptera frugiperda cells or in Trichoplusia larvae. Sequences encoding EP3-11 OR EP3-12 can be cloned into a non-essential region of the virus, such as the polyhedrin gene, and placed under control of the polyhedrin promoter. Successful insertion of EP3-11 OR EP3-12 will render the polyhedrin gene inactive and produce recombinant virus lacking coat protein. The recombinant viruses can then be used to infect S. frugiperda cells or Trichoplusia larvae in which EP3-11 OR EP3-12 can be expressed.

### Mammalian Expression Systems

A number of viral-based expression systems can be used to express EP3-11 OR EP3-12 in mammalian host cells. For example, if an adenovirus is used as an expression vector, sequences encoding EP3-11 OR EP3-12 can be ligated into an adenovirus transcription/-translation complex comprising the late promoter and tripartite leader sequence. Insertion in a non-essential E1 or E3 region of the viral genome can be used to obtain a viable virus which is capable of expressing EP3-11 OR EP3-12 in infected host cells. If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles). Specific initiation signals also can be used to achieve more efficient translation of sequences encoding EP3-11 OR EP3-12. Such signals include the ATG initiation codon and adjacent sequences. In cases where sequences encoding EP3-11 OR EP3-12, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic.

### Host Cells

A host cell strain can be chosen for its ability to modulate the expression of the inserted sequences or to process the expressed EP3-11 OR EP3-12 in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express EP3-11 OR EP3-12 can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced EP3-11 OR EP3-12 sequences. Resistant clones of stably transformed cells can be proliferated using tissue culture techniques appropriate to the cell type. Any number of selection systems can be used to recover transformed cell lines. These include, but are not limited to, the herpes simplex virus thymidine kinase and adenine phosphoribosyltransferase genes which can be employed in tk⁻ or aprt⁻ cells, respectively. Also, antimetabolite, antibiotic, or herbicide resistance can be used as the basis for selection. For example, dhfr confers resistance to methotrexate, npt confers resistance to the aminoglycosides, neomycin and G-418, and als and pat confer resistance to chlorsulfuron and phosphinotricin acetyltransferase, respectively. Additional selectable genes have been described. For example, trpB allows cells to utilize indole in place of tryptophan, or hisD, which allows cells to utilize histinol in place of histidine. Visible markers such as anthocyanins, β-glucuronidase and its substrate GUS, and luciferase and its substrate luciferin, can be used to identify transformants and to quantify the amount of transient or stable protein expression attributable to a specific vector system

### Detecting Polypeptide Expression

Although the presence of marker gene expression suggests that a EP3-11 OR EP3-12 polynucleotide is also present, its presence and expression may need to be confirmed. For example, if a sequence encoding EP3-11 OR EP3-12 is inserted within a marker gene sequence, transformed cells containing sequences which encode EP3-11 OR EP3-12 can be identified by the absence of marker gene function. Alternatively, a marker gene can be placed in tandem with a sequence encoding EP3-11 OR EP3-12 under the control of a single promoter. Expression of the marker gene in response to induction or selection usually indicates expression of EP3-11 OR EP3-12 polynucleotide.

Alternatively, host cells which contain a EP3-11 OR EP3-12 polynucleotide and which express EP3-11 OR EP3-12 can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a polynucleotide sequence encoding EP3-11 OR EP3-12 can be detected by DNA-DNA or DNA-RNA hybridization or amplification using probes or fragments or fragments of polynucleotides encoding EP3-11 OR EP3-12. Nucleic acid amplification-based assays involve the use of oligonucleotides selected from sequences encoding EP3-11 OR EP3-12 to detect transformants which contain a EP3-11 OR EP3-12 polynucleotide.

A variety of protocols for detecting and measuring the expression of EP3-11 OR EP3-12, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on EP3-11 OR EP3-12 can be used, or a competitive binding assay can be employed.

A wide variety of labels and conjugation techniques are known by those skilled in the art and can be used in various nucleic acid and amino acid assays. Means for producing labeled hybridization or PCR probes for detecting sequences related to polynucleotides encoding EP3-11 OR EP3-12 include oligolabeling, nick translation, end-labeling, or PCR amplification using a labeled nucleotide. Alternatively, sequences encoding EP3-11 OR EP3-12 can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

### Expression and Purification of Polypeptides

Host cells transformed with nucleotide sequences encoding EP3-11 OR EP3-12 can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode EP3-11 OR EP3-12 can be designed to contain signal sequences which direct secretion of soluble EP3-11 OR EP3-12 through a prokaryotic or eukaryotic cell membrane or which direct the membrane insertion of membrane-bound EP3-11 OR EP3-12.

As discussed above, other constructions can be used to join a sequence encoding EP3-11 OR EP3-12 to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor XA or enterokinase (Invitrogen, San Diego, Calif.) between the purification domain and EP3-11 OR EP3-12 also can be used to facilitate purification. One such expression vector provides for expression of a fusion protein containing EP3-11 OR EP3-12 and 6 histidine residues preceding a thioredoxin or an enterokinase cleavage site. The histidine residues facilitate purification by IMAC (immobilized metal ion affinity chromatography), while the enterokinase cleavage site provides a means for purifying EP3-11 OR EP3-12 from the fusion protein.

### Chemical Synthesis

Sequences encoding EP3-11 OR EP3-12 can be synthesized, in whole or in part, using chemical methods well known in the art. Alternatively, EP3-11 OR EP3-12 itself can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of EP3-11 OR EP3-12 can be separately synthesized and combined using chemical methods to produce a full-length molecule.

The newly synthesized peptide can be substantially purified by preparative high performance liquid chromatography. The composition of a synthetic EP3-11 OR EP3-12 can be confirmed by amino acid analysis or sequencing. Additionally, any portion of the amino acid sequence of EP3-11 OR EP3-12 can be altered during direct synthesis and/or combined using chemical methods with sequences from other proteins to produce a variant polypeptide or a fusion protein.

### Production of Altered Polypeptides

As will be understood by those of skill in the art, it may be advantageous to produce EP3-11 OR EP3-12-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences referred to herein can be engineered using methods generally known in the art to alter EP3-11 OR EP3-12-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

### Antibodies

Any type of antibody known in the art can be generated to bind specifically to an epitope of EP3-11 OR EP3-12. “Antibody” as used herein includes intact immunoglobulin molecules, as well as fragments thereof, such as Fab, F(ab′)₂, and Fv, which are capable of binding an epitope of EP3-11 OR EP3-12. Typically, at least 6, 8, 10, or 12 contiguous amino acids are required to form an epitope. However, epitopes which involve non-contiguous amino acids may require more, e.g., at least 15, 25, or 50 amino acid. An antibody which specifically binds to an epitope of EP3-11 OR EP3-12 can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to EP3-11 OR EP3-12 provides a detection signal at least 5-, 10-, or 20-fold higher than a detection signal provided with other proteins when used in an immunochemical assay. Preferably, antibodies which specifically bind to EP3-11 OR EP3-12 do not detect other proteins in immunochemical assays and can immunoprecipitate EP3-11 OR EP3-12 from solution.

EP3-11 OR EP3-12 can be used to immunize a mammal, such as a mouse, rat, rabbit, guinea pig, monkey, or human, to produce polyclonal antibodies. If desired, EP3-11 OR EP3-12 can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to EP3-11 OR EP3-12 can be prepared using any technique which provides for the production of antibody molecules by continuous cell lines in culture. These techniques include, but are not limited to, the hybridoma technique, the human B-cell hybridoma technique, and the EBV-hybridoma technique.

In addition, techniques developed for the production of “chimeric antibodies,” the splicing of mouse antibody genes to human antibody genes to obtain a molecule with appropriate antigen specificity and biological activity, can be used. Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, techniques described for the production of single chain antibodies can be adapted using methods known in the art to produce single chain antibodies which specifically bind to EP3-11 OR EP3-12. Antibodies with related specificity, but of distinct idiotypic composition, can be generated by chain shuffling from random combinatorial immunoglobin libraries. Single-chain antibodies also can be constructed using a DNA amplification method, such as PCR, using hybridoma cDNA as a template. Single-chain antibodies can be mono- or bispecific, and can be bivalent or tetravalent. Construction of tetravalent, bispecific single-chain antibodies is taught. A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage.

Antibodies which specifically bind to EP3-11 OR EP3-12 also can be produced by inducing in vivo production in the lymphocyte population or by screening immunoglobulin libraries or panels of highly specific binding reagents. Other types of antibodies can be constructed and used therapeutically in methods of the invention.

Antibodies according to the invention can be purified by methods well known in the art. For example, antibodies can be affinity purified by passage over a column to which EP3-11 OR EP3-12 is bound. The bound antibodies can then be eluted from the column using a buffer with a high salt concentration.

### Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of EP3-11 OR EP3-12 gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters.

Modifications of EP3-11 OR EP3-12 gene expression can be obtained by designing antisense oligonucleotides which will form duplexes to the control, 5′, or regulatory regions of the EP3-11 OR EP3-12 gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. An antisense oligonucleotide also can be designed to block translation of mRNA by preventing the transcript from binding to ribosomes.

Precise complementarity is not required for successful complex formation between an antisense oligonucleotide and the complementary sequence of a EP3-11 OR EP3-12 polynucleotide. Antisense oligonucleotides which comprise, for example, 2, 3, 4, or 5 or more stretches of contiguous nucleotides which are precisely complementary to a EP3-11 OR EP3-12 polynucleotide, each separated by a stretch of contiguous nucleotides which are not complementary to adjacent EP3-11 OR EP3-12 nucleotides, can provide sufficient targeting specificity for EP3-11 OR EP3-12 mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular EP3-11 OR EP3-12 polynucleotide sequence. Antisense oligonucleotides can be modified without affecting their ability to hybridize to a EP3-11 OR EP3-12 polynucleotide. These modifications can be internal or at one or both ends of the antisense molecule. For example, inter-nucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, also can be employed in a modified antisense oligonucleotide.

### Ribozymes

Ribozymes are RNA molecules with catalytic activity. Ribozymes can be used to inhibit gene function by cleaving an RNA sequence, as is known in the art. The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences. The coding sequence of a EP3-11 OR EP3-12 polynucleotide can be used to generate ribozymes which will specifically bind to mRNA transcribed from a EP3-11 OR EP3-12 polynucleotide. Methods of designing and constructing ribozymes which can cleave other RNA molecules in trans in a highly sequence specific manner have been developed and described in the art. For example, the cleavage activity of ribozymes can be targeted to specific RNAs by engineering a discrete “hybridization” region into the ribozyme. The hybridization region contains a sequence complementary to the target RNA and thus specifically hybridizes with the Specific ribozyme cleavage sites within a EP3-11 OR EP3-12 RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate EP3-11 OR EP3-12 RNA targets also can be evaluated by testing accessibility to hybridization with complementary oligonucleotides using ribonuclease protection assays. The nucleotide sequences shown in SEQ ID NO:20 or SEQ ID NO:21 and its complement provide sources of suitable hybridization region sequences. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease EP3-11 OR EP3-12 expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells. Ribozymes also can be engineered to provide an additional level of regulation, so that destruction of mRNA occurs only when both a ribozyme and a target gene are induced in the cells.

### Screening/Screening Assays

### Regulators

Regulators as used herein, refers to EP3-11 OR EP3-12 agonists and EP3-11 OR EP3-12 antagonists. Agonists of EP3-11 OR EP3-12 are molecules which, when bound to EP3-11 OR EP3-12, increase or prolong the activity of EP3-11 OR EP3-12. Agonists of EP3-11 OR EP3-12 include proteins, nucleic acids, carbohydrates, small molecules, or any other molecule which activate EP3-11 OR EP3-12. Antagonists of EP3-11 OR EP3-12 are molecules which, when bound to EP3-11 OR EP3-12, decrease the amount or the duration of the activity of EP3-11 OR EP3-12. Antagonists include proteins, nucleic acids, carbohydrates, antibodies, small molecules, or any other molecule which decrease the activity of EP3-11 OR EP3-12.

The term “modulate,” as it appears herein, refers to a change in the activity of EP3-11 OR EP3-12. For example, modulation may cause an increase or a decrease in protein activity, binding characteristics, or any other biological, functional, or immunological properties of EP3-11 OR EP3-12.

As used herein, the terms “specific binding” or “specifically binding” refer to that interaction between a protein or peptide and an agonist, an antibody, or an antagonist. The interaction is dependent upon the presence of a particular structure of the protein recognized by the binding molecule (i.e., the antigenic determinant or epitope). For example, if an antibody is specific for epitope “A,” the presence of a polypeptide containing the epitope A, or the presence of free unlabeled A, in a reaction containing free labeled A and the antibody will reduce the amount of labeled A that binds to the antibody.

The invention provides methods (also referred to herein as “screening assays”) for identifying compounds which can be used for the treatment of cardiovascular diseases, inflammation, reproduction disorders and cancer. The methods entail the identification of candidate or test compounds or agents (e.g., peptides, peptidomimetics, small molecules or other molecules) which bind to EP3-11 OR EP3-12 and/or have a stimulatory or inhibitory effect on the biological activity of EP3-11 OR EP3-12 or its expression and then determining which of these compounds have an effect on symptoms or diseases regarding the cardiovascular diseases, inflammation, reproduction disorders and cancer in an in vivo assay.

Candidate or test compounds or agents which bind to EP3-11 OR EP3-12 and/or have a stimulatory or inhibitory effect on the activity or the expression of EP3-11 OR EP3-12 are identified either in assays that employ cells which express EP3-11 OR EP3-12 on the cell surface (cell-based assays) or in assays with isolated EP3-11 OR EP3-12 (cell-free assays). The various assays can employ a variety of variants of EP3-11 OR EP3-12 (e.g., full-length EP3-11 OR EP3-12, a biologically active fragment of EP3-11 OR EP3-12, or a fusion protein which includes all or a portion of EP3-11 OR EP3-12). Moreover, EP3-11 OR EP3-12 can be derived from any suitable mammalian species (e.g., human EP3-11 OR EP3-12, rat EP3-11 OR EP3-12 or murine EP3-11 OR EP3-12). The assay can be a binding assay entailing direct or indirect measurement of the binding of a test compound or a known EP3-11 OR EP3-12 ligand to EP3-11 OR EP3-12. The assay can also be an activity assay entailing direct or indirect measurement of the activity of EP3-11 OR EP3-12. The assay can also be an expression assay entailing direct or indirect measurement of the expression of EP3-11 OR EP3-12 mRNA or EP3-11 OR EP3-12 protein. The various screening assays are combined with an in vivo assay entailing measuring the effect of the test compound on the symptoms of a cardiovascular diseases, inflammation, reproduction disorders and cancer.

In one embodiment, the invention provides assays for screening candidate or test compounds which bind to or modulate the activity of a membrane-bound (cell surface expressed) form of EP3-11 OR EP3-12. Such assays can employ full-length EP3-11 OR EP3-12, a biologically active fragment of EP3-11 OR EP3-12, or a fusion protein which includes all or a portion of EP3-11 OR EP3-12. As described in greater detail below, the test compound can be obtained by any suitable means, e.g., from conventional compound libraries. Determining the ability of the test compound to bind to a membrane-bound form of EP3-11 OR EP3-12 can be accomplished, for example, by coupling the test compound with a radioisotope or enzymatic label such that binding of the test compound to the EP3-11 OR EP3-12-expressing cell can be measured by detecting the labeled compound in a complex. For example, the test compound can be labeled with . . . ¹²⁵I, . . . ³⁵S, . . . ¹⁴C, or . . . ³H, either directly or indirectly, and the radioisotope detected by direct counting of radioemmission or by scintillation counting. Alternatively, the test compound can be enzymatically labeled with, for example, horseradish peroxidase, alkaline phosphatase, or luciferase, and the enzymatic label detected by determination of conversion of an appropriate substrate to product.

In a competitive binding format, the assay comprises contacting EP3-11 OR EP3-12-expressing cell with a known compound which binds to EP3-11 OR EP3-12 to form an assay mixture, contacting the assay mixture with a test compound, and determining the ability of the test compound to interact with the EP3-11 OR EP3-12-expressing cell, wherein determining the ability of the test compound to interact with the EP3-11 OR EP3-12-expressing cell comprises determining the ability of the test compound to preferentially bind the EP3-11 OR EP3-12-expressing cell as compared to the known compound.

In another embodiment, the assay is a cell-based assay comprising contacting a cell expressing a membrane-bound form of EP3-11 OR EP3-12 (e.g., full-length EP3-11 OR EP3-12, a biologically active fragment of EP3-11 OR EP3-12, or a fusion protein which includes all or a portion of EP3-11 OR EP3-12) expressed on the cell surface with a test compound and determining the ability of the test compound to modulate (e.g., stimulate or inhibit) the activity of the membrane-bound form of EP3-11 OR EP3-12. Determining the ability of the test compound to modulate the activity of the membrane-bound form of EP3-11 OR EP3-12 can be accomplished by any method suitable for measuring the activity of EP3-11 OR EP3-12, e.g., any method suitable for measuring the activity of a G-protein coupled receptor or other seven-transmembrane receptor (described in greater detail below). The activity of a seven-transmembrane receptor can be measured in a number of ways, not all of which are suitable for any given receptor. Among the measures of activity are: alteration in intracellular Ca.sup.2+ concentration, activation of phospholipase C, alteration in intracellular inositol triphosphate (IP.sub.3) concentration, alteration in intracellular diacylglycerol (DAG) concentration, and alteration in intracellular adenosine cyclic 3′,5′-monophosphate (cAMP) concentration.

Determining the ability of the test compound to modulate the activity of EP3-11 OR EP3-12 can be accomplished, for example, by determining the ability of EP3-11 OR EP3-12 to bind to or interact with a target molecule. The target molecule can be a molecule with which EP3-11 OR EP3-12 binds or interacts with in nature, for example, a molecule on the surface of a cell which expresses EP3-11 OR EP3-12, a molecule on the surface of a second cell, a molecule in the extracellular milieu, a molecule associated with the internal surface of a cell membrane or a cytoplasmic molecule. The target molecule can be a component of a signal transduction pathway which facilitates transduction of an extracellular signal (e.g., a signal generated by binding of a EP3-11 OR EP3-12 ligand, through the cell membrane and into the cell. The target molecule can be, for example, a second intracellular protein which has catalytic activity or a protein which facilitates the association of downstream signaling molecules with EP3-11 OR EP3-12.

Determining the ability of EP3-11 OR EP3-12 to bind to or interact with a target molecule can be accomplished by one of the methods described above for determining direct binding. In one embodiment, determining the ability of a polypeptide of the invention to bind to or interact with a target molecule can be accomplished by determining the activity of the target molecule. For example, the activity of the target molecule can be determined by detecting induction of a cellular second messenger of the target (e.g., intracellular Ca.sup.2+, diacylglycerol, IP3, etc.), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g., luciferase), or detecting a cellular response.

The present invention also includes cell-free assays. Such assays involve contacting a form of EP3-11 OR EP3-12 (e.g., full-length EP3-11 OR EP3-12, a biologically active fragment of EP3-11 OR EP3-12, or a fusion protein comprising all or a portion of EP3-11 OR EP3-12) with a test compound and determining the ability of the test compound to bind to EP3-11 OR EP3-12. Binding of the test compound to EP3-11 OR EP3-12 can be determined either directly or indirectly as described above. In one embodiment, the assay includes contacting EP3-11 OR EP3-12 with a known compound which binds EP3-11 OR EP3-12 to form an assay mixture, contacting the assay mixture with a test compound, and determining the ability of the test compound to interact with EP3-11 OR EP3-12, wherein determining the ability of the test compound to interact with EP3-11 OR EP3-12 comprises determining the ability of the test compound to preferentially bind to EP3-11 OR EP3-12 as compared to the known compound.

The cell-free assays of the present invention are amenable to use of either a membrane-bound form of EP3-11 OR EP3-12 or a soluble fragment thereof. In the case of cell-free assays comprising the membrane-bound form of the polypeptide, it may be desirable to utilize a solubilizing agent such that the membrane-bound form of the polypeptide is maintained in solution. Examples of such solubilizing agents include but are not limited to non-ionic detergents such as n-octylglucoside, n-dodecylglucoside, n-dodecylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamido-propyl)dimethylamminio]-2-hydroxy-1-propane sulfonate (CHAPSO), or N-dodecyl=N,N-dimethyl-3-ammonio-1-propane sulfonate.

In various embodiments of the above assay methods of the present invention, it may be desirable to immobilize EP3-11 OR EP3-12 (or a EP3-11 OR EP3-12 target molecule) to facilitate separation of complexed from uncomplexed forms of one or both of the proteins, as well as to accommodate automation of the assay. Binding of a test compound to EP3-11 OR EP3-12, or interaction of EP3-11 OR EP3-12 with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase (GST) fusion proteins or glutathione-S-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or EP3-11 OR EP3-12, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of EP3-11 OR EP3-12 can be determined using standard techniques.

Other techniques for immobilizing proteins on matrices can also be used in the screening assays of the invention. For example, either EP3-11 OR EP3-12 or its target molecule can be immobilized utilizing conjugation of biotin and streptavidin. Biotinylated polypeptide of the invention or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated plates (Pierce Chemical). Alternatively, antibodies reactive with EP3-11 OR EP3-12 or target molecules but which do not interfere with binding of the polypeptide of the invention to its target molecule can be derivatized to the wells of the plate, and unbound target or polypeptidede of the invention trapped in the wells by antibody conjugation. Methods for detecting such complexes, in addition to those described above for the GST-immobilized complexes, include immunodetection of complexes using antibodies reactive with EP3-11 OR EP3-12 or target molecule, as well as enzyme-linked assays which rely on detecting an enzymatic activity associated with EP3-11 OR EP3-12 or target molecule.

The screening assay can also involve monitoring the expression of EP3-11 OR EP3-12. For example, regulators of expression of EP3-11 OR EP3-12 can be identified in a method in which a cell is contacted with a candidate compound and the expression of EP3-11 OR EP3-12 protein or mRNA in the cell is determined. The level of expression of EP3-11 OR EP3-12 protein or mRNA the presence of the candidate compound is compared to the level of expression of EP3-11 OR EP3-12 protein or mRNA in the absence of the candidate compound. The candidate compound can then be identified as a regulator of expression of EP3-11 OR EP3-12 based on this comparison. For example, when expression of EP3-11 OR EP3-12 protein or mRNA protein is greater (statistically significantly greater) in the presence of the candidate compound than in its absence, the candidate compound is identified as a stimulator of EP3-11 OR EP3-12 protein or mRNA expression. Alternatively, when expression of EP3-11 OR EP3-12 protein or mRNA is less (statistically significantly less) in the presence of the candidate compound than in its absence, the candidate compound is identified as an inhibitor of EP3-11 OR EP3-12 protein or mRNA expression. The level of EP3-11 OR EP3-12 protein or mRNA expression in the cells can be determined by methods described below.

### Binding Assays

For binding assays, the test compound is preferably a small molecule which binds to and occupies the active site of EP3-11 OR EP3-12 receptor polypeptide, thereby making the ligand binding site inaccessible to substrate such that normal biological activity is prevented. Examples of such small molecules include, but are not limited to, small peptides or peptide-like molecules. Potential ligands which bind to a polypeptide of the invention include, but are not limited to, the natural ligands of known EP3-11 OR EP3-12 receptor GPCRs and analogues or derivatives thereof.

In binding assays, either the test compound or the EP3-11 OR EP3-12 receptor polypeptide can comprise a detectable label, such as a fluorescent, radioisotopic, chemiluminescent, or enzymatic label, such as horseradish peroxidase, alkaline phosphatase, or luciferase. Detection of a test compound which is bound to EP3-11 OR EP3-12 receptor polypeptide can then be accomplished, for example, by direct counting of radioemmission, by scintillation counting, or by determining conversion of an appropriate substrate to a detectable product. Alternatively, binding of a test compound to a EP3-11 OR EP3-12 receptor polypeptide can be determined without labeling either of the interactants. For example, a microphysiometer can be used to detect binding of a test compound with a EP3-11 OR EP3-12 receptor polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and EP3-11 OR EP3-12.

Determining the ability of a test compound to bind to EP3-11 OR EP3-12 also can be accomplished using a technology such as real-time Bimolecular Interaction Analysis (BIA). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a EP3-11 OR EP3-12-like polypeptide can be used as a “bait protein” in a two-hybrid assay or three-hybrid, to identify other proteins which bind to or interact with EP3-11 OR EP3-12 and modulate its activity.

The two-hybrid system is based on the modular nature of most transcription factors, which consist of separable DNA-binding and activation domains. Briefly, the assay utilizes two different DNA constructs. For example, in one construct, polynucleotide encoding EP3-11 OR EP3-12 can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with EP3-11 OR EP3-12.

It may be desirable to immobilize either the EP3-11 OR EP3-12 (or polynucleotide) or the test compound to facilitate separation of the bound form from unbound forms of one or both of the interactants, as well as to accommodate automation of the assay. Thus, either the EP3-11 OR EP3-12-like polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach EP3-11 OR EP3-12-like polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to EP3-11 OR EP3-12 (or a polynucleotide encoding for EP3-11 OR EP3-12) can be accomplished in any vessel suitable for containing the reactants. Examples of such vessels include microtiter plates, test tubes, and microcentrifuge tubes.

In one embodiment, EP3-11 OR EP3-12 is a fusion protein comprising a domain that allows binding of EP3-11 OR EP3-12 to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed EP3-11 OR EP3-12; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either EP3-11 OR EP3-12 (or a polynucleotide encoding EP3-11 OR EP3-12) or a test compound can be immobilized utilizing conjugation of biotin and streptavidin. Biotinylated EP3-11 OR EP3-12 (or a polynucleotide encoding biotinylated EP3-11 OR EP3-12) or test compounds can be prepared from biotin-NHS (N-hydroxysuccinimide) using techniques well known in the art (e.g., biotinylation kit, Pierce Chemicals, Rockford, Ill.) and immobilized in the wells of streptavidin-coated plates (Pierce Chemical). Alternatively, antibodies which specifically bind to EP3-11 OR EP3-12, polynucleotide, or a test compound, but which do not interfere with a desired binding site, such as the active site of EP3-11 OR EP3-12, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to EP3-11 OR EP3-12 receptor polypeptide or test compound, enzyme-linked assays which rely on detecting an activity of EP3-11 OR EP3-12 receptor polypeptide, and SDS gel electrophoresis under non-reducing conditions.

Screening for test compounds which bind to a EP3-11 OR EP3-12 receptor polypeptide or polynucleotide also can be carried out in an intact cell. Any cell which comprises a EP3-11 OR EP3-12 receptor polypeptide or polynucleotide can be used in a cell-based assay system. A EP3-11 OR EP3-12 receptor polynucleotide can be naturally occurring in the cell or can be introduced using techniques such as those described above. Binding of the test compound to EP3-11 OR EP3-12 or a polynucleotide encoding EP3-11 OR EP3-12 is determined as described above.

### Functional Assays

Test compounds can be tested for the ability to increase or decrease EP3-11 OR EP3-12 activity of a EP3-11 OR EP3-12 receptor polypeptide. The EP3-11 OR EP3-12 activity can be measured, for example, using methods described in the specific examples, below. EP3-11 OR EP3-12 activity can be measured after contacting either a purified EP3-11 OR EP3-12, a cell membrane preparation, or an intact cell with a test compound. A test compound which decreases EP3-11 OR EP3-12 activity by at least about 10, preferably about 50, more preferably about 75, 90, or 100% is identified as a potential agent for decreasing EP3-11 OR EP3-12 activity. A test compound which increases EP3-11 OR EP3-12 activity by at least about 10, preferably about 50, more preferably about 75, 90, or 100% is identified as a potential agent for increasing EP3-11 OR EP3-12 activity.

One such screening procedure involves the use of melanophores which are transfected to express EP3-11 OR EP3-12. Thus, for example, such an assay may be employed for screening for a compound which inhibits activation of the receptor polypeptide of the present invention by contacting the melanophore cells which encode the receptor with both the receptor ligand and a compound to be screened. Inhibition of the signal generated by the ligand indicates that a compound is a potential antagonist for the receptor, i.e., inhibits activation of the receptor. The screen may be employed for identifying a compound which activates the receptor by contacting such cells with compounds to be screened and determining whether each compound generates a signal, i.e., activates the receptor.

Other screening techniques include the use of cells which express EP3-11 OR EP3-12 (for example, transfected CHO cells) in a system which measures extracellular pH changes caused by receptor activation. For example, compounds may be contacted with a cell which expresses the receptor polypeptide of the present invention and a second messenger response, e.g., signal transduction or pH changes, can be measured to determine whether the potential compound activates or inhibits the receptor. Another such screening technique involves introducing RNA encoding EP3-11 OR EP3-12 into Xenopus oocytes to transiently express the receptor. The receptor oocytes can then be contacted with the receptor ligand and a compound to be screened, followed by detection of inhibition or activation of a calcium signal in the case of screening for compounds which are thought to inhibit activation of the receptor.

Another screening technique involves expressing EP3-11 OR EP3-12 in cells in which the receptor is linked to a phospholipase C or D. Such cells include endothelial cells, smooth muscle cells, embryonic kidney cells, etc. The screening may be accomplished as described above by quantifying the degree of activation of the receptor from changes in the phospholipase activity.

### Gene Expression

In another embodiment, test compounds which increase or decrease EP3-11 OR EP3-12 gene expression are identified. As used herein, the term “correlates with expression of a “polynucleotide” indicates that the detection of the presence of nucleic acids, the same or related to a nucleic acid sequence encoding EP3-11 OR EP3-12, by northern analysis or relative PCR is indicative of the presence of nucleic acids encoding EP3-11 OR EP3-12 in a sample, and thereby correlates with expression of the transcript from the polynucleotide encoding EP3-11 OR EP3-12. The term “microarray,” as used herein, refers to an array of distinct polynucleotides or oligonucleotides arrayed on a substrate, such as paper, nylon or any other type of membrane, filter, chip, glass slide, or any other suitable solid support. A EP3-11 OR EP3-12 polynucleotide is contacted with a test compound, and the expression of an RNA or polypeptide product of EP3-11 OR EP3-12 polynucleotide is determined. The level of expression of appropriate mRNA or polypeptide in the presence of the test compound is compared to the level of expression of mRNA or polypeptide in the absence of the test compound. The test compound can then be identified as a regulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of EP3-11 OR EP3-12 mRNA or polypeptide expression in the cells can be determined by methods well known in the art for detecting mRNA or polypeptide. Either qualitative or quantitative methods can be used. The presence of polypeptide products of EP3-11 OR EP3-12 receptor polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into EP3-11 OR EP3-12.

Such screening can be carried out either in a cell-free assay system or in an intact cell. Any cell which expresses EP3-11 OR EP3-12 polynucleotide can be used in a cell-based assay system. The EP3-11 OR EP3-12 polynucleotide can be naturally occurring in the cell or can be introduced using techniques such as those described above. Either a primary culture or an established cell line can be used.

### Test Compounds

Suitable test compounds for use in the screening assays of the invention can be obtained from any suitable source, e.g., conventional compound libraries. The test compounds can also be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds.

### Modeling of Regulators

Computer modeling and searching technologies permit identification of compounds, or the improvement of already identified compounds, that can modulate EP3-11 OR EP3-12 expression or activity. Having identified such a compound or composition, the active sites or regions are identified. Such active sites might typically be ligand binding sites, such as the interaction domain of the ligand with EP3-11 OR EP3-12. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intramolecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential EP3-11 OR EP3-12 modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

### Therapeutic Indications and Methods

It was found by the present applicant that EP3-11 OR EP3-12 is expressed in different human tissues.

### Cardiovascular Diseases

Heart failure is defined as a pathophysiological state in which an abnormality of cardiac function is responsible for the failure of the heart to pump blood at a rate commensurate with the requirement of the metabolizing tissue. It includes all forms of pumping failures such as high-output and low-output, acute and chronic, right-sided or left-sided, systolic or diastolic, independent of the underlying cause.

Myocardial infarction (MD is generally caused by an abrupt decrease in coronary blood flow that follows a thrombotic occlusion of a coronary artery previously narrowed by arteriosclerosis. MI prophylaxis (primary and secondary prevention) is included as well as the acute treatment of MI and the prevention of complications.

Ischemic diseases are conditions in which the coronary flow is restricted resulting in a perfusion which is inadequate to meet the myocardial requirement for oxygen. This group of diseases includes stable angina, unstable angina and asymptomatic ischemia.

Arrhythmias include all forms of atrial and ventricular tachyarrhythmia (atrial tachycardia, atrial flutter, atrial fibrillation, atrio-ventricular reentrant tachycardia, preexitation syndrome, ventricular tachycardia, ventricular flutter, ventricular fibrillation) as well as bradycardic forms of arrhythmias.

Hypertensive vascular diseases include primary as well as all kinds of secondary arterial hypertension (renal, endocrine, neurogenic, others). The genes may be used as drug targets for the treatment of hypertension as well as for the prevention of all complications.

Peripheral vascular diseases are defined as vascular diseases in which arterial and/or venous flow is reduced resulting in an imbalance between blood supply and tissue oxygen demand. It includes chronic peripheral arterial occlusive disease (PAOD), acute arterial thrombosis and embolism, inflammatory vascular disorders, Raynaud's phenomenon and venous disorders.

The EP3-11 OR EP3-12 receptor is highly expressed in different tissues cardiovascular tissues as vessels, heart and kidney. The expression in the above mentioned tissues suggests an association between EP3-11 OR EP3-12 and cardiovascular diseases.

Cardiovascular diseases which can be treated, include but are not limited to the following disorders of the heart and the vascular system: congestive heart failure, myocardial infarction, ischemic diseases of the heart, all kinds of atrial and ventricular arrhythmias, hypertensive vascular diseases and peripheral vascular diseases.

### Inflammatory Diseases

The human prostaglandin e2, ep3 is highly expressed in different tissues which are involved in inflammatory processes The expression in the above mentioned tissues demonstrates that the human prostaglandin e2, ep3 or mRNA can be utilized to diagnose of inflammatory diseases. Additionally the activity of the human prostaglandin e2, ep3 can be modulated to treat inflammatory diseases.

Inflammatory diseases comprise diseases triggered by cellular or non cellular mediators of the immune system or tissues causing the inflammation of body tissues and subsequently producing an acute or chronic inflammatory condition. Examples for such inflammatory diseases are hypersensitivity reactions of type I IV, for example but not limited to hypersensitivity diseases of the lung including asthma, atopic diseases, allergic rhinitis or conjunctivitis, angioedema of the lids, hereditary angioedema, antireceptor hypersensitivity reactions and autoimmune diseases, Hashimoto's thyroiditis, systemic lupus erythematosus, Goodpasture's syndrome, pemphigus, myasthenia gravis, Grave's and Raynaud's disease, type B insulin resistant diabetes, rheumatoid arthritis, psoriasis, Crohn's disease, scleroderma, mixed connective tissue disease, polymyositis, sarcoidosis, glomerulonephritis, acute or chronic host versus graft reactions.

### Reproduction

The human prostaglandin e2, ep3 is highly expressed in tissues of the reproduction system as uterus. The expression in the above mentioned tissues demonstrates that the human prostaglandin e2, ep3 or mRNA can be utilized to diagnose of reproduction disorders. Additionally the activity of the human prostaglandin e2, ep3 can be modulated to treat reproduction disorders.

Disorders of the male reproductive system include but are not limited to balanoposthitis, balanitis xerotica obliterans, phimosis, paraphimosis, erythroplasia of Queyrat, skin cancer of the penis, Bowen's and Paget's diseases, syphilis, herpes simplex infections, genital warts, molluscum contagiosum, priapism, peyronie's disease, benign prostatic hyperplasia (BPH), prostate cancer, prostatitis, testicular cancer, testicular torsion, inguinal hernia, epididymo orchitis, mumps, hydroceles, spermatoceles, or varicoceles.

Impotence (erectile dysfunction) may results from vascular impairment, neurologic disorders, drugs, abnormalities of the penis, or psychologic problems.

Examples of disorders of the female reproductive include premature menopause, pelvic pain, vaginitis, vulvitis, vulvovaginitis, pelvic inflammatory disease, fibroids, menstrual disorders (premenstrual syndrome (PMS), dysmenorrhea, amenorrhea, primary amenorrhea, secondary amenorrhea, menorrhagia, hypomenorrhea, polymenorrhea, oligomenorrhea, metrorrhagia, menometrorrhagia, Postmenopausal bleeding), bleeding caused by a physical disorder, dysfunctional uterine bleeding, polycystic ovary syndrome (Stein Leventhal syndrome), endometriosis, cancer of the uterus, cancer of the cervix, cancer of the ovaries, cancer of the vulva, cancer of the vagina, cancer of the fallopian tubes, hydatidiform mole,

Infertility may be caused by problems with sperm, ovulation, the fallopian tubes, and the cervix as well as unidentified factors.

Complications of pregnancy include miscarriage and stillbirth, ectopic pregnancy, anemia, Rh incompatibility, problems with the placenta, excessive vomiting, preeclampsia, eclampsia, and skin rashes (e.g. herpes gestationis, urticaria of pregnancy) as well as preterm labor and premature rupture of the membranes.

Breast disorders may be noncancerous (benign) or cancerous (malignant). Examples of breast disorders are but are not limited to breast pain, cysts, fibrocystic breast disease, fibrous lumps, nipple discharge, breast infection, breast cancer (ductal carcinoma, lobular carcinoma, medullary carcinoma, tubular carcinoma, and inflammatory breast cancer), Paget's disease of the nipple or Cystosarcoma phyllodes.

### Cancer Disorders

The human prostaglandin e2, ep3 is highly expressed in different cancer related tissues. The expression in the above mentioned tissues and in particular the differential expression between diseased tissue uterus tumor and healthy tissue uterus demonstrates that the human prostaglandin e2, ep3 or mRNA can be utilized to diagnose of cancer. Additionally the activity of the human prostaglandin e2, ep3 can be modulated to treat cancer.

Cancer disorders within the scope of the invention comprise any disease of an organ or tissue in mammals characterized by poorly controlled or uncontrolled multiplication of normal or abnormal cells in that tissue and its effect on the body as a whole. Cancer diseases within the scope of the invention comprise benign neoplasms, dysplasias, hyperplasias as well as neoplasms showing metastatic growth or any other transformations like e.g. leukoplakias which often precede a breakout of cancer. Cells and tissues are cancerous when they grow more rapidly than normal cells, displacing or spreading into the surrounding healthy tissue or any other tissues of the body described as metastatic growth, assume abnormal shapes and sizes, show changes in their nucleocytoplasmatic ratio, nuclear polychromasia, and finally may cease. Cancerous cells and tissues may affect the body as a whole when causing paraneoplastic syndromes or if cancer occurs within a vital organ or tissue, normal function will be impaired or halted, with possible fatal results. The ultimate involvement of a vital organ by cancer, either primary or metastatic, may lead to the death of the mammal affected. Cancer tends to spread, and the extent of its spread is usually related to an individual's chances of surviving the disease. Cancers are generally said to be in one of three stages of growth: early, or localized, when a tumor is still confined to the tissue of origin, or primary site; direct extension, where cancer cells from the tumour have invaded adjacent tissue or have spread only to regional lymph nodes; or metastasis, in which cancer cells have migrated to distant parts of the body from the primary site, via the blood or lymph systems, and have established secondary sites of infection. Cancer is said to be malignant because of its tendency to cause death if not treated. Benign tumors usually do not cause death, although they may if they interfere with a normal body function by virtue of their location, size, or paraneoplastic side effects. Hence benign tumors fall under the definition of cancer within the scope of the invention as well. In general, cancer cells divide at a higher rate than do normal cells, but the distinction between the growth of cancerous and normal tissues is not so much the rapidity of cell division in the former as it is the partial or complete loss of growth restraint in cancer cells and their failure to differentiate into a useful, limited tissue of the type that characterizes the functional equilibrium of growth of normal tissue. Cancer tissues may express certain molecular receptors and probably are influenced by the host's susceptibility and immunity and it is known that certain cancers of the breast and prostate, for example, are considered dependent on specific hormones for their existence. The term “cancer” under the scope of the invention is not limited to simple benign neoplasia but comprises any other benign and malign neoplasia like 1) Carcinoma, 2) Sarcoma, 3) Carcinosarcoma, 4) Cancers of the blood forming tissues, 5) tumors of nerve tissues including the brain, 6) cancer of skin cells. Cancer according to 1) occurs in epithelial tissues, which cover the outer body (the skin) and line mucous membranes and the inner cavitary structures of organs e.g. such as the breast, lung, the respiratory and gastrointestinal tracts, the endocrine glands, and the genitourinary system. Ductal or glandular elements may persist in epithelial tumors, as in adenocarcinomas like e.g. thyroid adenocarcinoma, gastric adenocarcinoma, uterine adenocarcinoma. Cancers of the pavement cell epithelium of the skin and of certain mucous membranes, such as e.g. cancers of the tongue, lip, larynx, urinary bladder, uterine cervix, or penis, may be termed epidermoid or squamous cell carcinomas of the respective tissues and are in the scope of the definition of cancer as well. Cancer according to 2) develops in connective tissues, including fibrous tissues, adipose (fat) tissues, muscle, blood vessels, bone, and cartilage like e.g. osteogenic sarcoma; liposarcoma, fibrosarcoma, synovial sarcoma. Cancer according to 3) is cancer that develops in both epithelial and connective tissue. Cancer disease within the scope of this definition may be primary or secondary, whereby primary indicates that the cancer originated in the tissue where it is found rather than was established as a secondary site through metastasis from another lesion. Cancers and tumor diseases within the scope of this definition may be benign or malign and may affect all anatomical structures of the body of a mammal. By example but not limited to they comprise cancers and tumor diseases of I) the bone marrow and bone marrow derived cells (leukemias), II) the endocrine and exocrine glands like e.g. thyroid, parathyroid, pituitary, adrenal glands, salivary glands, pancreas III) the breast, like e.g. benign or malignant tumors in the mammary glands of either a male or a female, the mammary ducts, adenocarcinoma, medullary carcinoma, comedo carcinoma, Paget's disease of the nipple, inflammatory carcinoma of the young woman, IV) the lung, V) the stomach, VI) the liver and spleen, VII) the small intestine, VIII) the colon, IX) the bone and its supportive and connective tissues like malignant or benign bone tumour, e.g. malignant osteogenic sarcoma, benign osteoma, cartilage tumors; like malignant chondrosarcoma or benign chondroma; bone marrow tumors like malignant myeloma or benign eosinophilic granuloma, as well as metastatic tumors from bone tissues at other locations of the body; X) the mouth, throat, larynx, and the esophagus, XI) the urinary bladder and the internal and external organs and structures of the urogenital system of male and female like ovaries, uterus, cervix of the uterus, testes, and prostate gland, XII) the prostate, XIII) the pancreas, like ductal carcinoma of the pancreas; XIV) the lymphatic tissue like lymphomas and other tumors of lymphoid origin, XV) the skin, XVI) cancers and tumor diseases of all anatomical structures belonging to the respiration and respiratory systems including thoracal muscles and linings, XVII) primary or secondary cancer of the lymph nodes XVIII) the tongue and of the bony structures of the hard palate or sinuses, XVIV) the mouth, cheeks, neck and salivary glands, XX) the blood vessels including the heart and their linings, XXI) the smooth or skeletal muscles and their ligaments and linings, XXII) the peripheral, the autonomous, the central nervous system including the cerebellum, XXIII) the adipose tissue.

### APPLICATIONS

The present invention provides for both prophylactic and therapeutic methods for cardiovascular diseases, inflammation, reproduction disorders and cancer.

The regulatory method of the invention involves contacting a cell with an agent that modulates one or more of the activities of EP3-11 OR EP3-12. An agent that modulates activity can be an agent as described herein, such as a nucleic acid or a protein, a naturally-occurring cognate ligand of the polypeptide, a peptide, a peptidomimetic, or any small molecule. In one embodiment, the agent stimulates one or more of the biological activities of EP3-11 OR EP3-12. Examples of such stimulatory agents include the active EP3-11 OR EP3-12 and nucleic acid molecules encoding a portion of EP3-11 OR EP3-12. In another embodiment, the agent inhibits one or more of the biological activities of EP3-11 OR EP3-12. Examples of such inhibitory agents include antisense nucleic acid molecules and antibodies. These regulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by unwanted expression or activity of EP3-11 OR EP3-12 or a protein in the EP3-11 OR EP3-12 signaling pathway. In one embodiment, the method involves administering an agent like any agent identified or being identifiable by a screening assay as described herein, or combination of such agents that modulate say upregulate or downregulate the expression or activity of EP3-11 OR EP3-12 or of any protein in the EP3-11 OR EP3-12 signaling pathway. In another embodiment, the method involves administering a regulator of EP3-11 OR EP3-12 as therapy to compensate for reduced or undesirably low expression or activity of EP3-11 OR EP3-12 or a protein in the EP3-11 OR EP3-12 signalling pathway.

Stimulation of activity or expression of EP3-11 OR EP3-12 is desirable in situations in which activity or expression is abnormally low and in which increased activity is likely to have a beneficial effect. Conversely, inhibition of activity or expression of EP3-11 OR EP3-12 is desirable in situations in which activity or expression of EP3-11 OR EP3-12 is abnormally high and in which decreasing its activity is likely to have a beneficial effect.

This invention is further illustrated by the following examples which should not be construed as limiting. The contents of all references, patents and published patent applications cited throughout this application are hereby incorporated by reference.

### Pharmaceutical Compositions

This invention further pertains to novel agents identified by the above-described screening assays and uses thereof for treatments as described herein.

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes pharmaceutical compositions comprising a regulator of EP3-11 OR EP3-12 expression or activity (and/or a regulator of the activity or expression of a protein in the EP3-11 OR EP3-12 signalling pathway) as well as methods for preparing such compositions by combining one or more such regulators and a pharmaceutically acceptable carrier. Also within the invention are pharmaceutical compositions comprising a regulator identified using the screening assays of the invention packaged with instructions for use. For regulators that are antagonists of EP3-11 OR EP3-12 activity or which reduce EP3-11 OR EP3-12 expression, the instructions would specify use of the pharmaceutical composition for treatment of cardiovascular diseases, inflammation, reproduction disorders and cancer. For regulators that are agonists of EP3-11 OR EP3-12 activity or increase EP3-11 OR EP3-12 expression, the instructions would specify use of the pharmaceutical composition for treatment of cardiovascular diseases, inflammation, reproduction disorders and cancer.

An antagonist of EP3-11 OR EP3-12 may be produced using methods which are generally known in the art. In particular, purified EP3-11 OR EP3-12 may be used to produce antibodies or to screen libraries of pharmaceutical agents to identify those which specifically bind EP3-11 OR EP3-12. Antibodies to EP3-11 OR EP3-12 may also be generated using methods that are well known in the art. Such antibodies may include, but are not limited to, polyclonal, monoclonal, chimeric, single chain antibodies, Fab fragments, and fragments produced by a Fab expression library. Neutralizing antibodies like those which inhibit dimer formation are especially preferred for therapeutic use.

In another embodiment of the invention, the polynucleotides encoding EP3-11 OR EP3-12, or any fragment or complement thereof, may be used for therapeutic purposes. In one aspect, the complement of the polynucleotide encoding EP3-11 OR EP3-12 may be used in situations in which it would be desirable to block the transcription of the mRNA. In particular, cells may be transformed with sequences complementary to polynucleotides encoding EP3-11 OR EP3-12. Thus, complementary molecules or fragments may be used to modulate EP3-11 OR EP3-12 activity, or to achieve regulation of gene function. Such technology is now well known in the art, and sense or antisense oligonucleotides or larger fragments can be designed from various locations along the coding or control regions of sequences encoding EP3-11 OR EP3-12.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors which will express nucleic acid sequence complementary to the polynucleotides of the gene encoding EP3-11 OR EP3-12.

Any of the therapeutic methods described above may be applied to any subject in need of such therapy, including, for example, mammals such as dogs, cats, cows, horses, rabbits, monkeys, and most preferably, humans.

An additional embodiment of the invention relates to the administration of a pharmaceutical composition containing EP3-11 OR EP3-12 in conjunction with a pharmaceutically acceptable carrier, for any of the therapeutic effects discussed above. Such pharmaceutical compositions may consist of EP3-11 OR EP3-12, antibodies to EP3-11 OR EP3-12, and mimetics, agonists, antagonists, or inhibitors of EP3-11 OR EP3-12. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin. Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration. For pharmaceutical compositions which include an antagonist of EP3-11 OR EP3-12 activity, a compound which reduces expression of EP3-11 OR EP3-12, or a compound which reduces expression or activity of a protein in the EP3-11 OR EP3-12 signaling pathway or any combination thereof, the instructions for administration will specify use of the composition for cardiovascular diseases, inflammation, reproduction disorders and cancer. For pharmaceutical compositions which include an agonist of EP3-11 OR EP3-12 activity, a compound which increases expression of EP3-11 OR EP3-12, or a compound which increases expression or activity of a protein in the EP3-11 OR EP3-12 signaling pathway or any combination thereof, the instructions for administration will specify use of the composition for cardiovascular diseases, inflammation, reproduction disorders and cancer.

### Diagnostics

In another embodiment, antibodies which specifically bind EP3-11 OR EP3-12 may be used for the diagnosis of disorders characterized by the expression of EP3-11 OR EP3-12, or in assays to monitor patients being treated with EP3-11 OR EP3-12 or agonists, antagonists, and inhibitors of EP3-11 OR EP3-12. Antibodies useful for diagnostic purposes may be prepared in the same manner as those described above for therapeutics. Diagnostic assays for EP3-11 OR EP3-12 include methods which utilize the antibody and a label to detect EP3-11 OR EP3-12 in human body fluids or in extracts of cells or tissues. The antibodies may be used with or without modification, and may be labeled by covalent or non-covalent joining with a reporter molecule. A wide variety of reporter molecules, several of which are described above, are known in the art and may be used.

A variety of protocols for measuring EP3-11 OR EP3-12, including ELISAs, RIAs, and FACS, are known in the art and provide a basis for diagnosing altered or abnormal levels of EP3-11 OR EP3-12 expression. Normal or standard values for EP3-11 OR EP3-12 expression are established by combining body fluids or cell extracts taken from normal mammalian subjects, preferably human, with antibody to EP3-11 OR EP3-12 under conditions suitable for complex formation The amount of standard complex formation may be quantified by various methods, preferably by photometric means. Quantities of EP3-11 OR EP3-12 expressed in subject samples from biopsied tissues are compared with the standard values. Deviation between standard and subject values establishes the parameters for diagnosing disease.

In another embodiment of the invention, the polynucleotides encoding EP3-11 OR EP3-12 may be used for diagnostic purposes. The polynucleotides which may be used include oligonucleotide sequences, complementary RNA and DNA molecules, and PNAs. The polynucleotides may be used to detect and quantitate gene expression in biopsied tissues in which expression of EP3-11 OR EP3-12 may be correlated with disease. The diagnostic assay may be used to distinguish between absence, presence, and excess expression of EP3-11 OR EP3-12, and to monitor regulation of EP3-11 OR EP3-12 levels during therapeutic intervention.

Polynucleotide sequences encoding EP3-11 OR EP3-12 may be used for the diagnosis of a cardiovascular diseases, inflammation, reproduction disorders and cancer disorder associated with expression of EP3-11 OR EP3-12. The polynucleotide sequences encoding EP3-11 OR EP3-12 may be used in Southern-, Northern-, or dot-blot analysis, or other membrane-based technologies; in PCR technologies; in dipstick, pin, and ELISA assays; and in microarrays utilizing fluids or tissues from patient biopsies to detect altered EP3-11 OR EP3-12 expression. Such qualitative or quantitative methods are well known in the art.

In a particular aspect, the nucleotide sequences encoding EP3-11 OR EP3-12 may be useful in assays that detect the presence of associated disorders, particularly those mentioned above. The nucleotide sequences encoding EP3-11 OR EP3-12 may be labeled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered from that of a comparable control sample, the nucleotide sequences have hybridized with nucleotide sequences in the sample, and the presence of altered levels of nucleotide sequences encoding EP3-11 OR EP3-12 in the sample indicates the presence of the associated disorder. Such assays may also be used to evaluate the efficacy of a particular therapeutic treatment regimen in animal studies, in clinical trials, or in monitoring the treatment of an individual patient.

In order to provide a basis for the diagnosis of cardiovascular diseases, inflammation, reproduction disorders and cancer associated with expression of EP3-11 OR EP3-12, a normal or standard profile for expression is established. This may be accomplished by combining body fluids or cell extracts taken from normal subjects, either animal or human, with a sequence, or a fragment thereof, encoding EP3-11 OR EP3-12, under conditions suitable for hybridization or amplification. Standard hybridization may be quantified by comparing the values obtained from normal subjects with values from an experiment in which a known amount of a substantially purified polynucleotide is used. Standard values obtained from normal samples may be compared with values obtained from samples from patients who are symptomatic for a disorder. Deviation from standard values is used to establish the presence of a disorder.

In another embodiment, one may use competitive drug screening assays in which neutralizing antibodies capable of binding EP3-11 OR EP3-12 specifically compete with a test compound for binding EP3-11 OR EP3-12. In this manner, antibodies can be used to detect the presence of any peptide which shares one or more antigenic determinants with EP3-11 OR EP3-12.

G-protein coupled receptors are ubiquitous in the mammalian host and are responsible for many biological functions, including many pathologies. Accordingly, it is desirable to find compounds and drugs which stimulate a G-protein coupled receptor on the one hand and which can inhibit the function of a G-protein coupled receptor on the other hand. For example, compounds which activate the G-protein coupled receptor may be employed for therapeutic purposes, such as the treatment of: asthma, Parkinson's disease, acute heart failure, urinary retention, and osteoporosis. In particular, compounds which activate the receptors of the present invention are useful in treating various cardiovascular ailments such as caused by the lack of pulmonary blood flow or hypertension. In addition these compounds may also be used in treating various physiological disorders relating to abnormal control of fluid and electrolyte homeostasis and in diseases associated with abnormal angiotensin-induced aldosterone secretion.

In general, compounds which inhibit activation of the G-protein coupled receptor may be employed for a variety of therapeutic purposes, for example, for the treatment of hypotension and/or hypertension, angina pectoris, myocardial infarction, ulcers, asthma, allergies, benign prostatic hypertrophy, and psychotic and neurological disorders including schizophrenia, manic excitement, depression, delirium, dementia or severe mental retardation, dyskinesias, such as Huntington's disease or Tourett's syndrome, among others. Compounds which inhibit G-protein coupled receptors have also been useful in reversing endogenous anorexia and in the control of bulimia.

### Biomarker

One of ordinary skill in the art knows several methods and devices for the detection and analysis of the markers of the instant invention. With regard to polypeptides or proteins in patient test samples, immunoassay devices and methods are often used. These devices and methods can utilize labelled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labelled molecule.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassay (RIAs), competitive binding assays, planar waveguide technology, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like. For an example of how this procedure is carried out on a machine, one can use the RAMP Biomedical device, called the Clinical Reader Sup™, which uses the fluorescent tag method, though the skilled artisan will know of many different machines and manual protocols to perform the same assay. Diluted whole blood is applied to the sample well. The red blood cells are retained in the sample pad, and the separated plasma migrates along the strip. Fluorescent dyed latex particles bind to the analyte and are immobilized at the detection zone. Additional particles are immobilized at the internal control zone. The fluorescence of the detection and internal control zones are measured on the RAMP Clinical Reader Sup™, and the ratio between these values is calculated. This ratio is used to determine the analyte concentration by interpolation from a lot-specific standard curve supplied by the manufacturer in each test kit for each assay.

The use of immobilized antibodies specific for the markers is also contemplated by the present invention and is well known by one of ordinary skill in the art. The antibodies could be immobilized onto a variety of solid supports, such as magnetic or chromatographic matrix particles, the surface of an assay place (such as microtiter wells), pieces of a solid substrate material (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a coloured spot.

The analysis of a plurality of markers may be carried out separately or simultaneously with one test sample. Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvageable tissue, the appropriateness of drug therapies, the effectiveness of various therapies, identification of the severity of the event, identification of the disease severity, and identification of the patient's outcome, including risk of future events.

An assay consisting of a combination of the markers referenced in the instant invention may be constructed to provide relevant information related to differential diagnosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out methods described within the instant invention to optimize clinical sensitivity or specificity in various clinical settings.

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different analytes. Such formats include protein microarrays, or “protein chips” and capillary devices.

Cardiac markers serve an important role in the early detection and monitoring of cardiovascular disease. Markers of disease are typically substances found in a bodily sample that can be easily measured. The measured amount can correlate to underlying disease pathophysiology, presence or absence of a current or imminent cardiac event, probability of a cardiac event in the future. In patients receiving treatment for their condition the measured amount will also correlate with responsiveness to therapy. Markers can include elevated levels of blood pressure, cholesterol, blood sugar, homocysteine and C— reactive protein (CRP). However, current markers, even in combination with other measurements or risk factors, do not adequately identify patients at risk, accurately detect events (i.e., heart attacks), or correlate with therapy. For example, half of patients do not have elevated serum cholesterol or other traditional risk factors.

### Biomarker Classes

EP3-11 OR EP3-12 could be used as a biomarker for cardiovascular diseases, inflammation, reproduction disorders and cancer in different classes:

Disease Biomarker: a biomarker that relates to a clinical outcome or measure of disease.

Efficacy Biomarker: a biomarker that reflects beneficial effect of a given treatment.

Staging Biomarker: a biomarker that distinguishes between different stages of a chronic disorder.

Surrogate Biomarker: a biomarker that is regarded as a valid substitute for a clinical outcomes measure.

Toxicity Biomarker: a biomarker that reports a toxicological effect of a drug on an in vitro or in vivo system.

Mechanism Biomarker: a biomarker that reports a downstream effect of a drug.

Target Biomarker: a biomarker that reports interaction of the drug with its target.

### Determination of a Therapeutically Effective Dose

The determination of a therapeutically effective dose is well within the capability of those skilled in the art. A therapeutically effective dose refers to that amount of active ingredient which increases or decreases EP3-11 OR EP3-12 activity relative to EP3-11 OR EP3-12 activity which occurs in the absence of the therapeutically effective dose. For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 micrograms to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

If the expression product is mRNA, the reagent is preferably an antisense oligonucleotide or a ribozyme. Polynucleotides which express antisense oligonucleotides or ribozymes can be introduced into cells by a variety of methods, as described above. Preferably, a reagent reduces expression of EP3-11 OR EP3-12 gene or the activity of EP3-11 OR EP3-12 by at least about 10, preferably about 50, more preferably about 75, 90, or 100% relative to the absence of the reagent. The effectiveness of the mechanism chosen to decrease the level of expression of EP3-11 OR EP3-12 gene or the activity of EP3-11 OR EP3-12 can be assessed using methods well known in the art, such as hybridization of nucleotide probes to EP3-11 OR EP3-12-specific mRNA, quantitative RT-PCR, immunologic detection of EP3-11 OR EP3-12, or measurement of EP3-11 OR EP3-12 activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects. Any of the therapeutic methods described above can be applied to any subject in need of such therapy, including, for example, mammals such as dogs, cats, cows, horses, rabbits, monkeys, and most preferably, humans.

A “EP3-11 polynucleotide”, within the meaning of the invention, shall be understood as being a nucleic acid molecule selected from a group consisting of (i) a nucleic acid molecules encoding a polypeptide comprising the amino acid sequence of SEQ ID NO: 22, (ii) nucleic acid molecules comprising the sequence of SEQ ID NO: 20 or SEQ ID NO: 16, (iii) nucleic acid molecules having the sequence of SEQ ID NO: 20 or SEQ ID NO: 16, (iv) nucleic acid molecules of which the complementary strand hybridizes under stringent conditions to a nucleic acid molecule of (i), (ii), or (iii), (v) nucleic acid molecules of which the sequence differs from the sequence of a nucleic acid molecule of (iii) due to the degeneracy of the genetic code, and (vi) nucleic acid molecules which have a sequence identity of at least 80%, 85%, 90%, 95%, 98% or 99% to SEQ ID NO: 20 or SEQ ID NO: 16; and wherein the polypeptide encoded by said nucleic acid molecules of (i)-(vi) have EP3-11 activity.

In a preferred embodiment of the invention the aforementioned EP3-11 polynucleotide is not SEQ ID NO: 18.

A “EP3-12 polynucleotide”, within the meaning of the invention, shall be understood as being a nucleic acid molecule selected from a group consisting of (i) nucleic acid molecules encoding a polypeptide comprising the amino acid sequence of SEQ ID NO: 23, (ii) nucleic acid molecules comprising the sequence of SEQ ID NO: 21 or SEQ ID NO: 17, (iii) nucleic acid molecules having the sequence of SEQ ID NO: 21 or SEQ ID NO: 17, (iv) nucleic acid molecules of which the complementary strand hybridizes under stringent conditions to a nucleic acid molecule of (i), (ii), or (iii), (v) nucleic acid molecules of which the sequence differs from the sequence of a nucleic acid molecule of (iii) due to the degeneracy of the genetic code, and (vi) nucleic acid molecules which have a sequence identity of at least 80%, 85%, 90%, 95%, 98% or 99% to SEQ ID NO: 21 or SEQ ID NO: 17; and wherein the polypeptide encoded by said nucleic acid molecules of (i)-(vi) have EP3-12 activity.

In a preferred embodiment of the invention the aforementioned EP3-12 polynucleotide is not SEQ ID NO: 19.

Polypeptides of the invention are those polypeptides which are contained in a group of polypeptides consisting of (i) polypeptides having the sequence of SEQ ID NO:22 or SEQ ID NO:23, (ii) polypeptides comprising the sequence of SEQ ID NO:22 or SEQ ID NO:23, (iii) polypeptides encoded by nucleic acid molecules of the invention and (iv) polypeptides which show at least 99%, 98%, 95%, 90%, or 80% identity with a polypeptide of (i), (ii), or (iii), wherein said purified polypeptide has EP3-11 OR EP3-12 activity.

It is an objective of the invention to provide a vector comprising the nucleic acid molecule of the invention.

Another object of the invention is a host cell containing a vector of the invention.

Another object of the invention is a method of producing a EP3-11 OR EP3-12 comprising the steps of (i) culturing a host cell of the invention under suitable conditions and (ii) recovering the EP3-11 OR EP3-12 from the culture medium.

Another object of the invention is a method for the detection of a polynucleotide encoding a EP3-11 OR EP3-12 in a sample comprising the steps of (i) hybridizing a polynucleotide of the invention to nucleic acid material of the sample, thereby forming a hybridization complex; and (ii) detecting said hybridization complex.

Another object of the invention is a method for the detection of a polynucleotide encoding a EP3-11 OR EP3-12 in a sample comprising the steps of (i) hybridizing a polynucleotide of the invention to nucleic acid material of the sample, thereby forming a hybridization complex; and (ii) detecting said hybridization complex, wherein, before hybridization, the nucleic acid material of the sample is amplified.

Another object of the invention is a method for the detection of a polynucleotide of the invention or a polypeptide of the invention comprising the steps of (i) contacting a sample with a reagent which specifically interacts with a polynucleotide of the invention or a polypeptide of the invention, and (ii) detecting said interaction.

Another object of the invention are diagnostic kits for conducting any of the methods above.

Regulators of a given protein, within the meaning of the invention, are understood as being compounds which alter either directly or indirectly the activity of the given protein either in vivo or in vitro. Alteration of the activity can be, e.g., but not limited to, by allosteric effects or by affecting the expression of the given protein.

Other objects of the invention are methods for screening for regulators of the activity of a EP3-11 OR EP3-12 comprising the steps of (i) contacting a test compound with a polypeptide of the invention, (ii) detect binding of said test compound to said polypeptide of the invention, wherein test compounds that bind under (ii) are identified as potential regulators of the EP3-11 OR EP3-12 activity.

Other objects of the invention are methods of the above, wherein the step of contacting is in or at the surface of a cell.

Other objects of the invention are methods of the above, wherein the step of contacting is in or at the surface of a cell wherein the cell is in vitro.

Other objects of the invention are methods of the above, wherein the step of contacting is in a cell-free system.

Other objects of the invention are methods of the above, wherein the polypeptide of the invention is coupled to a detectable label.

Other objects of the invention are methods of the above, wherein the compound is coupled to a detectable label.

Other objects of the invention are methods of the above, wherein the test compound displaces a ligand which is first bound to the polypeptide.

Other objects of the invention are methods of the above, wherein the polypeptide of the invention is attached to a solid support.

Other objects of the invention are methods of the above, wherein the compound is attached a solid support.

Another object of the invention is a method of screening for regulators of the activity of a EP3-11 OR EP3-12 comprising the steps of

(i) measuring the activity of a polypeptide of the invention at a certain concentration of a test compound or in the absence of said test compound, (ii) measuring the activity of said polypeptide at a different concentrations of said test compound, wherein said test compound is identified as a regulator of the activity of a EP3-11 OR EP3-12 when there is a significant difference between the activities measured in (i) and (ii).

Another object of the invention is a method of screening for regulators of the activity of a EP3-11 OR EP3-12 comprising the steps of (i) measuring the activity of a polypeptide of the invention at a certain concentration of a test compound, (ii) measuring the activity of a polypeptide of the invention at the presence of a compound known to be a regulator of EP3-11 OR EP3-12.

Another object of the invention is a method of screening for regulators of the activity of a EP3-11 OR EP3-12 comprising the aforementioned methods, wherein the activities are measured in a cell.

Another object of the invention is a method of screening for regulators of the activity of a EP3-11 OR EP3-12 comprising the aforementioned methods, wherein the cell is in vitro.

Another object of the invention is a method of screening for regulators of the activity of a EP3-11 OR EP3-12 comprising the aforementioned methods, wherein the activities are measured in a cell-free system.

Another object of the invention is a method of screening for regulators of EP3-11 OR EP3-12 comprising the steps of (i) contacting a test compound with a nucleic acid molecule of the invention, (ii) detect binding of said test compound to said nucleic acid molecule, wherein said test compound is identified as a potential regulator of EP3-11 OR EP3-12 when it binds to said nucleic acid molecule.

Another object of the invention is a method of screening for regulators of EP3-11 OR EP3-12 comprising the steps of (i) contacting a test compound with a nucleic acid molecule of the invention, wherein the nucleic acid molecule is an RNA (ii) detect binding of said test compound to said RNA molecule, wherein said test compound is identified as a potential regulator of EP3-11 OR EP3-12 when it binds to said RNA molecule.

Another object of the invention is a method of screening for regulators of EP3-11 OR EP3-12 comprising the steps of contacting a test compound with a nucleic acid molecule of the invention, detect binding of said test compound to said nucleic acid molecule, wherein said test compound is identified as a potential regulator of EP3-11 OR EP3-12 when it binds to said nucleic acid molecule, wherein the contacting step is (i) in or at the surface of a cell or (ii) in a cell-free system or wherein (iii) the polypeptide or nucleic acid molecule is coupled to a detectable label or wherein (iv) the test compound is coupled to a detectable label.

Another object of the invention is a method of regulating the activity of a EP3-11 OR EP3-12 wherein EP3-11 OR EP3-12 is contacted with a regulator of EP3-11 OR EP3-12.

Another object of the invention is a method of diagnosing a EP3-11 OR EP3-12 related disease in a diseased mammal comprising the steps of (i) measuring the amount of a nucleic acid molecule of the invention in a sample taken from said diseased mammal, (ii) comparing the result of (i) to the amount of said nucleic acid molecule in one or several healthy mammals, wherein a EP3-11 OR EP3-12 related disease is diagnosed in the diseased mammal when the amount of said nucleic acid molecule in the diseased mammal is significantly different from the amount of said nucleic acid molecule in the healthy mammal/mammals.

Other objects of the invention are pharmaceutical compositions comprising (i) a nucleic acid molecule of the invention, (ii) a vector of the invention, or (iii) a polypeptide of the invention.

Another object of the invention are pharmaceutical compositions comprising a regulator of the invention.

Another object of the invention are pharmaceutical compositions comprising a regulator identified by methods of the invention for the treatment of cardiovascular diseases, inflammation, reproduction disorders and cancer in a mammal.

Another object of the invention regards the use of regulators of a EP3-11 OR EP3-12 as identified by any of the aforementioned methods for the preparation of pharmaceutical compositions useful for the treatment of cardiovascular diseases, inflammation, reproduction disorders and cancer in a mammal.

Another object of the invention are methods for the preparation of pharmaceutical compositions useful for the treatment of cardiovascular diseases, inflammation, reproduction disorders and cancer in a mammal comprising the steps of (i) identifying a regulator of EP3-11 OR EP3-12 by any of the before mentioned methods, (ii) determining of whether said regulator ameliorates the symptoms of cardiovascular diseases, inflammation, reproduction disorders and cancer in a mammal, (iii) combining of said regulator with an acceptable pharmaceutical carrier.

Another object of the invention is the use of a regulator of EP3-11 OR EP3-12 as identified by any of the aforementioned methods for (i) the treatment of cardiovascular diseases, inflammation, reproduction disorders and cancer in a mammal, or (ii) use of a regulator of EP3-11 OR EP3-12 for the regulation of EP3-11 OR EP3-12 activity in a mammal having a cardiovascular diseases, inflammation, reproduction disorders and cancer.

Another object of the invention is the use of any of the aforementioned pharmaceutical compositions wherein the regulator of EP3-11 OR EP3-12 is either a small molecule, an RNA molecule, or an antisense oligonucleotide, or a polypeptide, an antibody, or a ribozyme. Small molecules, within the meaning of the invention, are organic molecules of a molecular weight of less than one thousand five hundred grams per mol.

The examples below are provided to illustrate the subject invention. These examples are provided by way of illustration and are not included for the purpose of limiting the invention.

### EXAMPLES

### Example 1

### Expression Profiling

Total cellular RNA was isolated from cells by one of two standard methods: 1) guanidine isothiocyanate/Cesium chloride density gradient centrifugation; or with the Tri-Reagent protocol according to the manufacturer's specifications (Molecular Research Center, Inc., Cincinnati, Ohio). Total RNA prepared by the Tri-reagent protocol was treated with DNAse I to remove genomic DNA contamination.

For relative quantitation of the mRNA distribution of EP3-11 OR EP3-12, total RNA from each cell or tissue source was first reverse transcribed. 85 μg of total RNA was reverse transcribed using 1 μmole random hexamer primers, 0.5 mM each of dATP, dCTP, dGTP and dTTP (Qiagen, Hilden, Germany), 3000 U RnaseQut (Invitrogen, Groningen, Netherlands) in a final volume of 680 μl. The first strand synthesis buffer and Omniscript reverse transcriptase (2 u/μl) were from (Qiagen, Hilden, Germany). The reaction was incubated at 37° C. for 90 minutes and cooled on ice. The volume was adjusted to 6800 μl with water, yielding a final concentration of 12.5 ng/μl of starting RNA.

For relative quantitation of the distribution of EP3-11 OR EP3-12 mRNA in cells and tissues the Perkin Elmer ABI Prism®. 7700 Sequence Detection system or Biorad iCycler was used according to the manufacturer's specifications and protocols. PCR reactions were set up to quantitate EP3-11 OR EP3-12 and the housekeeping genes HPRT (hypoxanthine phosphoribosyltransferase), GAPDH (glyceraldehyde-3-phosphate dehydrogenase), β-actin, and others. Forward and reverse primers and probes for EP3-11 OR EP3-12 were designed using the Perkin Elmer ABI Primer Express™ software and were synthesized by TibMolBiol (Berlin, Germany). The EP3-11 forward primer sequence was: Primer 1a (SEQ ID NO:54). The EP3-11 reverse primer sequence was Primer 2a (SEQ ID NO:55). Probe 1a (SEQ ID NO:56), labelled with FAM (carboxyfluorescein succinimidyl ester) as the reporter dye and TAMRA (carboxytetramethylrhodamine) as the quencher, is used as a probe for EP3-11. The EP3-12 forward primer sequence was: Primer 1b (SEQ ID NO:57). The EP3-12 reverse primer sequence was Primer 2b (SEQ ID NO:58). Probe 1b (SEQ ID NO:59), labelled with FAM (carboxyfluorescein succinimidyl ester) as the reporter dye and TAMRA (carboxytetramethylrhodamine) as the quencher, is used as a probe for EP3-12. The following reagents were prepared in a total of 25 μl: 1× TaqMan buffer A, 5.5 mM MgCl₂, 200 nM of dATP, dCTP, dGTP, and dUTP, 0.025 U/μl AmpliTaq Gold™, 0.01 U/μl AmpErase and Probe 1a or 1b, EP3-11 OR EP3-12 forward and reverse primers each at 200 nM, 200 nM EP3-11 OR EP3-12 FAM/TAMRA-labelled probe, and 5 μl of template cDNA. Thermal cycling parameters were 2 min at 50° C., followed by 10 min at 95° C., followed by 40 cycles of melting at 95° C. for 15 sec and annealing/extending at 60° C. for 1 min.

The expression profile of EP3-11 is shown in FIG. 34. EP3-11 is specifically expressed in heart, aorta, artery, bronchia, penis, kidney, stomach tumor, colon tumor, rectum tumor, adipose tissue, skin, uterus, uterus tumor and breast.

The expression profile of EP3-12 is shown in FIG. 35. EP3-12 is specifically expressed in aorta, artery, heart valves, cerebral cortex, bronchia, adrenal gland, stomach, stomach tumor, small intestine, rectum, adipose tissue, uterus, uterus tumor and fetal tissues.

For relative quantitation of the distribution of EP3-1 to EP3-10 and EP3-13 to EP3-14 mRNA in cells and tissues the following primer and probe combinations were used: EP3-1: SEQ ID NO: 24, SEQ ID NO: 25, SEQ ID NO: 26; EP3-2: SEQ ID NO: 27, SEQ ID NO: 28, SEQ ID NO: 29; EP3-3: SEQ ID NO: 30, SEQ ID NO: 31, SEQ ID NO: 32, EP3-4: SEQ ID NO: 33, SEQ ID NO: 34, SEQ ID NO: 35; EP3-5: SEQ ID NO: 36, SEQ ID NO: 37, SEQ ID NO: 38; EP3-6: SEQ ID NO: 39, SEQ ID NO: 39, SEQ ID NO: 40; EP3-7: SEQ ID NO: 42, SEQ ID NO: 43, SEQ ID NO: 44; EP3-8: SEQ ID NO: 45, SEQ ID NO: 46, SEQ ID NO: 47; EP3-9: SEQ ID NO: 48, SEQ ID NO: 49, SEQ ID NO: 50; EP3-10: SEQ ID NO: 51, SEQ ID NO: 52, SEQ ID NO: 53; EP3-13: SEQ ID NO: 60, SEQ ID NO: 61, SEQ ID NO: 62 and EP3-14: SEQ ID NO: 63, SEQ ID NO: 64, SEQ ID NO: 65.

### Calculation of Corrected CT Values

The CT (threshold cycle) value is calculated as described in the “Quantitative determination of nucleic acids” section. The CF-value (factor for threshold cycle correction) is calculated as follows:
1. PCR reactions were set up to quantitate the housekeeping genes (HKG) for each cDNA sample.
2. CT_{HKG}-values (threshold cycle for housekeeping gene) were calculated as described in the “Quantitative determination of nucleic acids” section.
3. CT_{HKG}-mean values (CT mean value of all HKG tested on one cDNAs) of all HKG for each cDNA are calculated (n=number of HKG):

CT_{HKG}-mean value=(CT_{HKG1}-value+CT_{HKG2}-value+ . . . +CT_{HKG-n}-value)/n
4. CTₚₐₙₙₑₗ mean value (CT mean value of all HKG in all tested cDNAs)=(CT_{HKG1}-mean value+CT_{HKG2}-mean value+ . . . +CT_{HKG-y}-mean value)/y
   (y=number of cDNAs)
5. CF_{cDNA-n} (correction factor for cDNA n)=CTₚₐₙₙₑₗ-mean value−CT_{HKG-n}-mean value
6. CT_{cDNA-n} (CT value of the tested gene for the cDNA n)+CF_{cDNA-n} (correction factor for cDNA n)=CT_{cor-cDNA-n} (corrected CT value for a gene on cDNA n)

### Calculation of Relative Expression

Definition: highest CT_{cor-cDNA-n}≠40 is defined as CT_{cor-cDNA}[high]
Relative Expression=2^{(CTcor-cDNA[high]-CTcor-cDNA-n)}

### Example 2

### Expression of EP3-11 OR EP3-12

Expression of EP3-11 OR EP3-12 is accomplished by subcloning the cDNAs into appropriate expression vectors and transfecting the vectors into expression hosts such as, e.g., E. coli. In a particular case, the vector is engineered such that it contains a promoter for β-galactosidase, upstream of the cloning site, followed by sequence containing the amino-terminal Methionine and the subsequent seven residues of β-galactosidase. Immediately following these eight residues is an engineered bacteriophage promoter useful for artificial priming and transcription and for providing a number of unique endonuclease restriction sites for cloning.

Induction of the isolated, transfected bacterial strain with Isopropyl-β-D-thiogalactopyranoside (IPTG) using standard methods produces a fusion protein corresponding to the first seven residues of β-galactosidase, about 15 residues of “linker”, and the peptide encoded within the cDNA. Since cDNA clone inserts are generated by an essentially random process, there is probability of 33% that the included cDNA will lie in the correct reading frame for proper translation. If the cDNA is not in the proper reading frame, it is obtained by deletion or insertion of the appropriate number of bases using well known methods including in vitro mutagenesis, digestion with exonuclease III or mung bean nuclease, or the inclusion of an oligonucleotide linker of appropriate length.

The EP3-11 OR EP3-12 cDNA is shuttled into other vectors known to be useful for expression of proteins in specific hosts. Oligonucleotide primers containing cloning sites as well as a segment of DNA (about 25 bases) sufficient to hybridize to stretches at both ends of the target cDNA is synthesized chemically by standard methods. These primers are then used to amplify the desired gene segment by PCR. The resulting gene segment is digested with appropriate restriction enzymes under standard conditions and isolated by gel electrophoresis. Alternately, similar gene segments are produced by digestion of the cDNA with appropriate restriction enzymes. Using appropriate primers, segments of coding sequence from more than one gene are ligated together and cloned in appropriate vectors. It is possible to optimize expression by construction of such chimeric sequences.

Suitable expression hosts for such chimeric molecules include, but are not limited to, mammalian cells such as Chinese Hamster Ovary (CHO) and human 293 cells, insect cells such as Sf9 cells, yeast cells such as Saccharomyces cerevisiae and bacterial cells such as E. coli. For each of these cell systems, a useful expression vector also includes an origin of replication to allow propagation in bacteria, and a selectable marker such as the β-lactamase antibiotic resistance gene to allow plasmid selection in bacteria. In addition, the vector may include a second selectable marker such as the neomycin phosphotransferase gene to allow selection in transfected eukaryotic host cells. Vectors for use in eukaryotic expression hosts require RNA processing elements such as 3′ polyadenylation sequences if such are not part of the cDNA of interest.

Additionally, the vector contains promoters or enhancers which increase gene expression. Such promoters are host specific and include MMTV, SV40, and metallothionine promoters for CHO cells; trp, lac, tac and T7 promoters for bacterial hosts; and alpha factor, alcohol oxidase and PGH promoters for yeast. Transcription enhancers, such as the rows sarcoma virus enhancer, are used in mammalian host cells. Once homogeneous cultures of recombinant cells are obtained through standard culture methods, large quantities of recombinantly produced EP3-11 OR EP3-12 are recovered from the conditioned medium and analyzed using chromatographic methods known in the art. For example, EP3-11 OR EP3-12 can be cloned into the expression vector pcDNA3, as exemplified herein. This product can be used to transform, for example, HEK293 or COS by methodology standard in the art. Specifically, for example, using Lipofectamine (Gibco BRL catolog no. 18324-020) mediated gene transfer.

### Example 3

### Isolation of Recombinant EP3-11 OR EP3-12

EP3-11 OR EP3-12 is expressed as a chimeric protein with one or more additional polypeptide domains added to facilitate protein purification. Such purification facilitating domains include, but are not limited to, metal chelating peptides such as histidine-tryptophan modules that allow purification on immobilized metals and the domain utilized in the FLAGS extension/affinity purification system (Immunex Corp., Seattle, Wash.). The inclusion of a cleavable linker sequence such as Factor Xa or enterokinase (Invitrogen, Groningen, The Netherlands) between the purification domain and the EP3-11 OR EP3-12 sequence is useful to facilitate expression of EP3-11 OR EP3-12.

### Example 4

### Testing of Chimeric GPCRs

Functional chimeric GPCRs are constructed by combining the extracellular receptive sequences of a new isoform with the transmembrane and intracellular segments of a known isoform for test purposes. This concept was demonstrated by Kobilka [Kobilka et al. 1988] who created a series of chimeric α2-β2 adrenergic receptors (AR) by inserting progressively greater amounts of α2-AR transmembrane sequence into β2-AR. The binding activity of known agonists changed as the molecule shifted from having more α2 than β2 conformation, and intermediate constructs demonstrated mixed specificity. The specificity for binding antagonists, however, correlated with the source of the domain VII. The importance of T7G domain VII for ligand recognition was also found in chimeras utilizing two yeast α-factor receptors and is significant because the yeast receptors are classified as miscellaneous receptors. Thus, functional role of specific domains appears to be preserved throughout the GPCR family regardless of category.

In parallel fashion, internal segments or cytoplasmic domains from a particular isoform are exchanged with the analogous domains of a known GPCRs and used to identify the structural determinants responsible for coupling the receptors to trimeric G-proteins. A chimeric receptor in which domains V, VI, and the intracellular connecting loop from β2-AR were substituted into α2-AR was shown to bind ligands with α2-AR specificity, but to stimulate adenylate cyclase in the manner of β2-AR. This demonstrates that for adrenergic-type receptors, G-protein recognition is present in domains V and VI and their connecting loop. The opposite situation was predicted and observed for a chimera in which the V->VI loop from α1-AR replaced the corresponding domain on β2-AR and the resulting receptor bound ligands with β2-AR specificity and activated G-protein-mediated phosphatidylinositol turnover in the α1-AR manner. Finally, chimeras constructed from muscarinic receptors also demonstrated that V->VI loop is the major determinant for specificity of G-protein activity.

Chimeric or modified GPCRs containing substitutions in the extracellular and transmembrane regions have shown that these portions of the receptor determine ligand binding specificity. For example, two Serine residues conserved in domain V of all adrenergic and D catecholainine GPCRs are necessary for potent agonist activity. These serines are believed to form hydrogen bonds with the catechol moiety of the agonists within the GPCR binding site. Similarly, an Asp residue present in domain III of all GPCRs which bind biogenic amines is believed to form an ion pair with the ligand amine group in the GPCR binding site.

Functional, cloned GPCRs are expressed in heterologous expression systems and their biological activity assessed. One heterologous system introduces genes for a mammalian GPCR and a mammalian G-protein into yeast cells. The GPCR is shown to have appropriate ligand specificity and affinity and trigger appropriate biological activation (growth arrest and morphological changes) of the yeast cells.

An alternate procedure for testing chimeric receptors is based on the procedure utilizing the purinergic receptor (P₂u). Function is easily tested in cultured K562 human leukemia cells because these cells lack P₂u receptors. K562 cells are transfected with expression vectors containing either normal or chimeric P₂u and loaded with fura-a, fluorescent probe for Ca⁺⁺. Activation of properly assembled and functional P₂u receptors with extracellular UTP or ATP mobilizes intracellular Ca⁺⁺ which reacts with fura-a and is measured spectrofluorometrically.

As with the GPCRs above, chimeric genes are created by combining sequences for extracellular receptive segments of any new GPCR polypeptide with the nucleotides for the transmembrane and intracellular segments of the known P₂u molecule. Bathing the transfected K562 cells in microwells containing appropriate ligands triggers binding and fluorescent activity defining effectors of the GPCR molecule. Once ligand and function are established, the P₂u system is useful for defining antagonists or inhibitors which block binding and prevent such fluorescent reactions.

### Example 5

### Production of EP3-11 OR EP3-12 Specific Antibodies

Two approaches are utilized to raise antibodies to EP3-11 OR EP3-12, and each approach is useful for generating either polyclonal or monoclonal antibodies. In one approach, denatured protein from reverse phase HPLC separation is obtained in quantities up to 75 mg. This denatured protein is used to immunize mice or rabbits using standard protocols; about 100 μg are adequate for immunization of a mouse, while up to 1 mg might be used to immunize a rabbit. For identifying mouse hybridomas, the denatured protein is radioiodinated and used to screen potential murine B-cell hybridomas for those which produce antibody. This procedure requires only small quantities of protein, such that 20 mg is sufficient for labeling and screening of several thousand clones.

In the second approach, the amino acid sequence of an appropriate EP3-11 OR EP3-12 domain, as deduced from translation of the cDNA, is analyzed to determine regions of high antigenicity. Oligopeptides comprising appropriate hydrophilic regions are synthesized and used in suitable immunization protocols to raise antibodies. The optimal amino acid sequences for immunization are usually at the C-terminus, the N-terminus and those intervening, hydrophilic regions of the polypeptide which are likely to be exposed to the external environment when the protein is in its natural conformation.

Typically, selected peptides, about 15 residues in length, are synthesized using an Applied Biosystems Peptide Synthesizer Model 431A using fmoc-chemistry and coupled to keyhole limpet hemocyanin (KLH; Sigma, St. Louis, Mo.) by reaction with M-maleimidobenzoyl-N-hydroxysuccinimide ester, MBS. If necessary, a cysteine is introduced at the N-terminus of the peptide to permit coupling to KLH. Rabbits are immunized with the peptide-KLH complex in complete Freund's adjuvant. The resulting antisera are tested for antipeptide activity by binding the peptide to plastic, blocking with 1% bovine serum albumin, reacting with antisera, washing and reacting with labeled (radioactive or fluorescent), affinity purified, specific goat anti-rabbit IgG.

Hybridomas are prepared and screened using standard techniques. Hybridomas of interest are detected by screening with labeled EP3-11 OR EP3-12 to identify those fusions producing the monoclonal antibody with the desired specificity. In a typical protocol, wells of plates (FAST; Becton-Dickinson, Palo Alto, Calif.) are coated during incubation with affinity purified, specific rabbit anti-mouse (or suitable antispecies 1 g) antibodies at 10 mg/ml. The coated wells are blocked with 1% bovine serum albumin, (BSA), washed and incubated with supernatants from hybridomas. After washing the wells are incubated with labeled EP3-11 OR EP3-12 at 1 mg/ml. Supernatants with specific antibodies bind more labeled EP3-11 OR EP3-12 than is detectable in the background. Then clones producing specific antibodies are expanded and subjected to two cycles of cloning at limiting dilution. Cloned hybridomas are injected into pristane-treated mice to produce ascites, and monoclonal antibody is purified from mouse ascitic fluid by affinity chromatography on Protein A. Monoclonal antibodies with affinities of at least 10⁸ M⁻¹, preferably 10⁹ to 10¹⁰ M⁻¹ or stronger, are typically made by standard procedures.

### Example 6

### Diagnostic Test Using EP3-11 OR EP3-12 Specific Antibodies

Particular EP3-11 OR EP3-12 antibodies are useful for investigating signal transduction and the diagnosis of infectious or hereditary conditions which are characterized by differences in the amount or distribution of EP3-11 OR EP3-12 or downstream products of an active signaling cascade.

Diagnostic tests for EP3-11 OR EP3-12 include methods utilizing antibody and a label to detect EP3-11 OR EP3-12 in human body fluids, membranes, cells, tissues or extracts of such. The polypeptides and antibodies of the present invention are used with or without modification. Frequently, the polypeptides and antibodies are labeled by joining them, either covalently or noncovalently, with a substance which provides for a detectable signal. A wide variety of labels and conjugation techniques are known and have been reported extensively in both the scientific and patent literature. Suitable labels include radionuclides, enzymes, substrates, cofactors, inhibitors, fluorescent agents, chemiluminescent agents, chromogenic agents, magnetic particles and the like.

A variety of protocols for measuring soluble or membrane-bound EP3-11 OR EP3-12, using either polyclonal or monoclonal antibodies specific for the protein, are known in the art. Examples include enzyme-linked immunosorbent assay (ELISA), radioimmunoassay (MA) and fluorescent activated cell sorting (FACS). A two-site monoclonal-based immunoassay utilizing monoclonal antibodies reactive to two non-interfering epitopes on EP3-11 OR EP3-12 is preferred, but a competitive binding assay may be employed.

### Example 7

### Drug Screening

This invention is particularly useful for screening therapeutic compounds by using EP3-11 OR EP3-12 or binding fragments thereof in any of a variety of drug screening techniques. As EP3-11 OR EP3-12 is a G protein coupled receptor any of the methods commonly used in the art may potentially be used to identify EP3-11 OR EP3-12 ligands. For example, the activity of a G protein coupled receptor such as EP3-11 OR EP3-12 can be measured using any of a variety of appropriate functional assays in which activation of the receptor results in an observable change in the level of some second messenger system, such as adenylate cyclase, guanylylcyclase, calcium mobilization, or inositol phospholipid hydrolysis. Alternatively, the polypeptide or fragment employed in such a test is either free in solution, affixed to a solid support, borne on a cell surface or located intracellularly. One method of drug screening utilizes eukaryotic or prokaryotic host cells which are stably transformed with recombinant nucleic acids expressing the polypeptide or fragment. Drugs are screened against such transformed cells in competitive binding assays. Such cells, either in viable or fixed form, are used for standard binding assays.

Measured, for example, is the formation of complexes between EP3-11 OR EP3-12 and the agent being tested. Alternatively, one examines the diminution in complex formation between EP3-11 OR EP3-12 and a ligand caused by the agent being tested.

Thus, the present invention provides methods of screening for drug candidates, drugs, or any other agents which affect signal transduction. These methods, well known in the art, comprise contacting such an agent with EP3-11 OR EP3-12 polypeptide or a fragment thereof and assaying (i) for the presence of a complex between the agent and EP3-11 OR EP3-12 polypeptide or fragment, or (ii) for the presence of a complex between EP3-11 OR EP3-12 polypeptide or fragment and the cell. In such competitive binding assays, the EP3-11 OR EP3-12 polypeptide or fragment is typically labeled. After suitable incubation, free EP3-11 OR EP3-12 polypeptide or fragment is separated from that present in bound form, and the amount of free or uncomplexed label is a measure of the ability of the particular agent to bind to EP3-11 OR EP3-12 or to interfere with the EP3-11 OR EP3-12-agent complex.

Another technique for drug screening provides high throughput screening for compounds having suitable binding affinity to EP3-11 OR EP3-12 polypeptides. Briefly stated, large numbers of different small peptide test compounds are synthesized on a solid substrate, such as plastic pins or some other surface. The peptide test compounds are reacted with EP3-11 OR EP3-12 polypeptide and washed. Bound EP3-11 OR EP3-12 polypeptide is then detected by methods well known in the art. Purified EP3-11 OR EP3-12 are also coated directly onto plates for use in the aforementioned drug screening techniques. In addition, non-neutralizing antibodies are used to capture the peptide and immobilize it on the solid support.

This invention also contemplates the use of competitive drug screening assays in which neutralizing antibodies capable of binding EP3-11 OR EP3-12 specifically compete with a test compound for binding to EP3-11 OR EP3-12 polypeptides or fragments thereof. In this manner, the antibodies are used to detect the presence of any peptide which shares one or more antigenic determinants with EP3-11 OR EP3-12.

### Example 8

### Rational Drug Design

The goal of rational drug design is to produce structural analogs of biologically active polypeptides of interest or of small molecules with which they interact, agonists, antagonists, or inhibitors. Any of these examples are used to fashion drugs which are more active or stable forms of the polypeptide or which enhance or interfere with the function of a polypeptide in vivo.

In one approach, the three-dimensional structure of a protein of interest, or of a protein-inhibitor complex, is determined by x-ray crystallography, by computer modeling or, most typically, by a combination of the two approaches. Both the shape and charges of the polypeptide must be ascertained to elucidate the structure and to determine active site(s) of the molecule. Less often, useful information regarding the structure of a polypeptide is gained by modeling based on the structure of homologous proteins. In both cases, relevant structural information is used to design efficient inhibitors. Useful examples of rational drug design include molecules which have improved activity or stability or which act as inhibitors, agonists, or antagonists of native peptides.

It is also possible to isolate a target-specific antibody, selected by functional assay, as described above, and then to solve its crystal structure. This approach, in principle, yields a pharmacore upon which subsequent drug design is based. It is possible to bypass protein crystallography altogether by generating anti-idiotypic antibodies (anti-ids) to a functional, pharmacologically active antibody. As a mirror image of a mirror image, the binding site of the anti-ids is expected to be an analog of the original receptor. The anti-id is then used to identify and isolate peptides from banks of chemically or biologically produced peptides. The isolated peptides then act as the pharmacore.

By virtue of the present invention, sufficient amount of polypeptide are made available to perform such analytical studies as X-ray crystallography. In addition, knowledge of the EP3-11 OR EP3-12 amino acid sequence provided herein provides guidance to those employing computer modeling techniques in place of or in addition to x-ray crystallography.

### Example 9

### Use and Administration of Antibodies, Inhibitors, or Antagonists

Antibodies, inhibitors, or antagonists of EP3-11 OR EP3-12 or other treatments and compounds that are limiters of signal transduction (LSTs), provide different effects when administered therapeutically. LSTs are formulated in a nontoxic, inert, pharmaceutically acceptable aqueous carrier medium preferably at a pH of about 5 to 8, more preferably 6 to 8, although pH may vary according to the characteristics of the antibody, inhibitor, or antagonist being formulated and the condition to be treated. Characteristics of LSTs include solubility of the molecule, its half-life and antigenicity/immunogenicity. These and other characteristics aid in defining an effective carrier. Native human proteins are preferred as LSTs, but organic or synthetic molecules resulting from drug screens are equally effective in particular situations.

LSTs are delivered by known routes of administration including but not limited to topical creams and gels; transmucosal spray and aerosol; transdermal patch and bandage; injectable, intravenous and lavage formulations; and orally administered liquids and pills particularly formulated to resist stomach acid and enzymes. The particular formulation, exact dosage, and route of administration is determined by the attending physician and varies according to each specific situation.

Such determinations are made by considering multiple variables such as the condition to be treated, the LST to be administered, and the pharmacokinetic profile of a particular LST. Additional factors which are taken into account include severity of the disease state, patient's age, weight, gender and diet, time and frequency of LST administration, possible combination with other drugs, reaction sensitivities, and tolerance/response to therapy. Long acting LST formulations might be administered every 3 to 4 days, every week, or once every two weeks depending on half-life and clearance rate of the particular LST.

Normal dosage amounts vary from 0.1 to 10⁵ μg, up to a total dose of about 1 g, depending upon the route of administration. Guidance as to particular dosages and methods of delivery is provided in the literature. Those skilled in the art employ different formulations for different LSTs. Administration to cells such as nerve cells necessitates delivery in a manner different from that to other cells such as vascular endothelial cells.

It is contemplated that abnormal signal transduction, trauma, or diseases which trigger EP3-11 OR EP3-12 activity are treatable with LSTs. These conditions or diseases are specifically diagnosed by the tests discussed above, and such testing should be performed in suspected cases of viral, bacterial or fungal infections, allergic responses, mechanical injury associated with trauma, hereditary diseases, lymphoma or carcinoma, or other conditions which activate the genes of lymphoid or neuronal tissues.

### REFERENCES

Adam, M.; Boie, Y.; Rushmore, T. H.; Muller, G.; Bastien, L.; McKee, K. T.; Metters, K. M.; Abramovitz, M, FEBS Lett. 338: 170-174, 1994.
Barnes, P. J. Mechanisms in COPD. Differences from asthma. Chest 2000, 117:10 S14S
Kobilka et al., Science 240:1310-1316, 1988
Kotani, M.; Tanaka, I.; Ogawa, Y.; Usui, T.; Mori, K.; Ichikawa, A.; Narumiya, S.; Yoshimi, T.; Nakao, K. Molec. Pharm. 48: 869-879, 1995
Kotani, M.; Tanaka, I.; Ogawa, Y.; Suganami, T.; Matsumoto, T.; Muro, S.; Yamamoto, Y.; Sugawara, A.; Yoshimasa, Y.; Sagawa, N.; Narumiya, S.; Nakao, K. J. Clin. Endocr. Metab. 85: 4315-4322, 2000
Gibson, U. E. M., Heid, C. A. and Williams, P. M., Genome Research. 6: 995-1001, 1996.
Heid, C. A., Stevens, J., Livak, K. J., and Williams, P. M., Genome Research. 6: 986-994, 1996.
Holland, P. M., Abramson, R. D., Watson, R. and Gelfand, D. H., PNAS. 88: 7276-7280, 1991.
Jeffreys A J, W. V., Thein S L, Nature. 316: 76-9, 1985.
Lefkowitz, Nature 351, 353-354, 1991
Maniatis et al., Molecular Cloning: A Laboratory Manual, 1982, Cold Spring Harbor Laboratory, Cold Spring Harbor Press
Piatak, M. J., et al., BioTechniques 14:70-81 (1993)
Weber et al., Genomics. 7: 524-30, 1990.
Ushikubi, F.; Segi, E.; Sugimoto, Y.; Murata, T.; Matsuoka, T.; Kobayashi, T.; Hizaki, H.; Tuboi, K.; Katsuyama, M.; Ichikawa, A.; Tanaka, T.; Yoshida, N.; Narumiya, S, Nature 395: 281-284, 1998
WO 03/064471
U.S. Pat. No. 4,683,195
U.S. Pat. No. 4,800,195
U.S. Pat. No. 4,965,188

1. A nucleic acid molecule selected from a group consisting of
i) nucleic acid molecules encoding a polypeptide comprising the amino acid sequence of SEQ ID NO:22 or 23,
ii) nucleic acid molecules comprising the sequence of SEQ ID NO: 16, 17, 20 or 21,
iii) nucleic acid molecules having the sequence of SEQ ID NO:20 or 21,
iv) nucleic acid molecules the complementary strand of which hybridizes under stringent conditions to a nucleic acid molecule of (i), (ii), or (iii); and
v) nucleic acid molecules the sequence of which differs from the sequence of a nucleic acid molecule of (iii) due to the degeneracy of the genetic code;
wherein the polypeptide encoded by said nucleic acid molecule has EP3-11 or EP3-12 activity.

2. A purified polypeptide selected from a group consisting of
i) polypeptides having the sequence of SEQ ID NO:22 or 23,
ii) polypeptides comprising the sequence of SEQ ID NO:22 or 23,
iii) polypeptides encoded by nucleic acid molecules of claim 1; and
iv) polypeptides which show at least 99%, 98%, 95%, 90%, or 80% identity with a polypeptide of (i), (ii), or (iii);
wherein said purified polypeptide has EP3-11 or EP3-12 activity.

3. A vector comprising the nucleic acid molecule of claim 1.

4. A host cell containing the vector of claim 3.

5. (canceled)

6. A method for the detection of a polynucleotide of claim 1 comprising the steps of
i) contacting a sample with a reagent which specifically interacts with a polynucleotide of claim 1; and
ii) detecting said interaction.

7. A method for screening for regulators of the activity of a EP3-11 or EP3-12 comprising the steps of
i) contacting a test compound with a polypeptide of claim 2,
ii) detect binding of said test compound to said polypeptide of claim 2,
wherein test compounds that bind under (ii) are identified as potential regulators of the EP3-11 or EP3-12 activity.

8. A method of screening for regulators of the activity of a EP3-11 or EP3-12 comprising the steps of
i) measuring the activity of a polypeptide of claim 2 at a certain concentration of a test compound or in the absence of said test compound,
ii) measuring the activity of said polypeptide at a different concentration of said test compound,
wherein said test compound is identified as a regulator of the activity of a EP3-11 or EP3-12 when there is a significant difference between the activities measured in (i) and (ii).

9. A method of screening for regulators of the activity of a EP3-11 or EP3-12 comprising the steps of
i) measuring the activity of a polypeptide of claim 2 at a certain concentration of a test compound,
ii) measuring the activity of a polypeptide of claim 2 at the presence of a compound known to be a regulator of EP3-11 or EP3-12.

10. A method of screening for regulators of EP3-11 or EP3-12 comprising the steps of
i) contacting a test compound with a nucleic acid molecule of claim 1,
ii) detect binding of said test compound to said nucleic acid molecule,
wherein said test compound is identified as a potential regulator of EP3-11 or EP3-12 when it binds to said nucleic acid molecule.

11. A method of regulating the activity of a EP3-11 or EP3-12 wherein EP3-11 or EP3-12 is contacted with a regulator of EP3-11 or EP3-12.

12. A method of diagnosing a EP3-11 or EP3-12 related disease in a diseased mammal comprising the steps of
i) measuring the amount of a nucleic acid molecule of claim 1 in a sample taken from said diseased mammal,
ii) comparing the result of (i) to the amount of said nucleic acid molecule in one or several healthy mammals,
wherein a EP3-11 or EP3-12 related disease is diagnosed in the diseased mammal when the amount of said nucleic acid molecule in the diseased mammal is significantly different from the amount of said nucleic acid molecule in the healthy mammal/mammals.

13. A pharmaceutical composition comprising a nucleic acid molecule of claim 1.

14. A pharmaceutical composition comprising a vector of claim 3.

15. A pharmaceutical composition comprising a polypeptide of claim 2.

16-22. (canceled)

23. A method for the detection of a polypeptide of claim 2 comprising the steps of
i) contacting a sample with a reagent which specifically interacts with a polypeptide of claim 2; and
ii) detecting said interaction.


SEQUENCE LISTING (65 sequences)

SEQ ID NO: 1 - DNA, 1137 bp, Homo Sapiens
gcgcctcgccaccagaggtttcccagagaggaaggcgtggctccctcccgggccagtgagccctggcgccgccgcggccgcggtcccagcagcggagtagggcggcggctgcgccccgcaccatggggggcagcccagccccagccgcggtaaacgccgacctccgccgccgcccgcgccgcgtctgccccctcccgctgcggctctctggacgccatcccctcctcacctcgaagccaacatgaaggagacccggggctacggaggggatgcccccttctgcacccgcctcaaccactcctacacaggcatgtgggcgcccgagcgttccgccgaggcgcggggcaacctcacgcgccctccagggtctggcgaggattgcggatcggtgtccgtggccttcccgatcaccatgctgctcactggtttcgtgggcacgcactggccatgctgctcgtgtcgcgcagctaccggcgccgggagagcaagcgcaagaagtccttcctgctgtgcatcggctggctggcgctcaccgacctggtcgggcagcttctcaccaccccggtcgtcatcgtcgtgtacctgtccaagcagcgttgggagcacatcgacccgtcggggcggctctgcacctttttcgggctgaccatgactgttttcgggctctcctcgttgttcatcgccagcgccatggccgtcgagcgggcgctggccatcagggcgccgcactggtatgcgagccacatgaagacgcgtgccacccgcgctgtgctgctcggcgtgtggctggccgtgctcgccttcgccctgctgccggtgctgggcgtgggccagtacaccgtccagtggcccgggacgtggtgcttcatcagcaccgggcgagggggcaacgggactagctcttcgcataactggggcaaccttttcttcgcctctgcctttgccttcctggggctcttggcgctgacagtcaccttttcctgcaacctggccaccattaaggccctggtgtcccgctgccgggccaaggccacggcatctcagtccagtgcccagtggggccgcatcacgaccgagacggccattcagcttatggggatcatgtgcgtgctgtcggtctgctggtctccgctcctg

SEQ ID NO: 2 - DNA, 180 bp, Homo Sapiens
ataatgatgttgaaaatgatcttcaatcagacatcagttgagcactgcaagacacacacggagaagcagaaagaatgcaacttcttcttaatagctgttcgcctggcttcactgaaccagatcttggatccttgggtttacctgctgttaagaaagatccttcttcgaaagttttgccag

SEQ ID NO: 3 - DNA, 92 bp, Homo Sapiens
atcaggtaccacacaaacaactatgcatccagctccacctccttaccctgccagtgttcctcaaccttgatgtggagcgaccatttggaaag

SEQ ID NO: 4 - DNA, 948 bp, Homo Sapiens
ataatgaaagaacggagttggacattttattgcaattcctgcttccctgaatttgcatatttcttcccacctgagaaggataattatatattttaatttggattatttcttcatttttatctttttattttaatgattgttttgtcagtaatacccatggagatcaactttattattataatccatgcctctgaatattagattggtttcttggatgggattttgaatatgcatttaagaagttgggaagaatttcacagatgatgattggaggaaaagtgatgaaaagaaagacctgtgttccaggagttttctccaacttcaaacctttacgtgaatcttaaccaaagtggacatctttacatttcatgatagcttgcttttgcaatatgagtttgaaaaatcagtataagcttatgatggtgaaaagtcaacatattgagagtgataattcaattaataggatatgaacttaacgacatataaaagcaaatgagggcaggagggaatcgtgacaaaaaatatttgtgcccaattatgattaatgttttagagatgttgtgtccctgtgggcttagcatggaactaaaagttcctaagtctcaattctagttatgtgtcatttagtaactcaggaatctgcttaattgttatttctaagcttttgatgacaaaggagtgatgcagctaagggcatccttggagtgtcataaaaaacaaatttgaggttgaatgattagctgcgcagttagagtgataaaaaaatccaggtaggccttctgattcaccatgaccaagtcaggatttcttaatatttcttttctggcagcatatacaaaggcaaaattaataaataacagttgttgaataacaaactttattacgtttttataaaataaaagaatctattttgtctgtattaaaataaaaaactttgtggacttctaa

SEQ ID NO: 5 - DNA, 27 bp, Homo Sapiens
atgagaaaaagaagactcagagagcaa

SEQ ID NO: 6 - DNA, 627 bp, Homo Sapiens
ttaatctgcagtttgcgaactctcagatacagagggcaactgcacattgtgggcaagtacaaacctatcgtgtgctgaacagagaagaaatggaggtactcgtgagcagcattaatgtgtacaccagaatctcaacagtaaagacagaataacatgaagaaaataactacaggagtgctgtgttttaaaaaagcaagctcctccagctgagtttgtgactctaatccctgcttattttgagcactttccatgtgttgattgactcggaaaggtgggagattaccagcaagccaggtcatttgggcaaacatgggcatatttttagcaaagctgaacaaactggttggtcactcgaactacccaatttagacttatagtaagaatagcacatggttcagaattgccaagactgctgctttagtcaacagaaattttttcatatgcatgctggtaatgcagtgcatctctggaagaaatgcaagagtattttaacatttggtccagaagaaatatgtaaagggtgtttgtagaatttgatgccctcttcagtgacataaggttatgggtttgtgaatggaaaatctgttatttgttcaggttattaaaacacattctaattcaacatca

SEQ ID NO: 7 - DNA, 51 bp, Homo Sapiens
ttctggaggccggcaagttcaggatcagggtgccagcagattcggtgtctg

SEQ ID NO: 8 - DNA, 366 bp, Homo Sapiens
atggagtcttgctctctcacccagactggagtgcagtggagtgatttccgctcactgcaaccttcacctcctccactcactgcaatcttcgcctcctgggttcaagtgattctcctgcctcagcctcccaagtagctggaattgcaggcatgcgccacaagcctggctaatttttgcatttttagtagagatgaggtttcaccatgtttgccaggctggtcttgaacacctgacctcaagtaacccacccaccttggcctcccaaagagctgggattacaggcatgagccaacgtgcctggccatgttctgatcgtttaatgatagcaacatttagtattatagagcatgaaaatgtcaaagtcgc

SEQ ID NO: 9 - DNA, 76 bp, Homo Sapiens
gagatggggcctgatggaaggtgtttttgtcatgcatggaggcaggtccccaggacttggtgcagttctcatgata

SEQ ID NO: 10 - DNA, 145 bp, Homo Sapiens
gctcctcttcttcccacctctactgtgattgatccttcaaggttctgtgctcagcccttccgttggttcttggatttgtcctttcccgccatgtcttcatcacatccacaacttccactaacacttgcgagcttcaaacttctta

SEQ ID NO: 11 - DNA, 93 bp, Homo Sapiens
gagaaccctgcagtgtccagctaagctgatgacttgaagataaatctgcctaaccctgggatgaagtatctgtgaactattttgacagcagat

SEQ ID NO: 12 - DNA, 507 bp, Homo Sapiens
gaggaattttggggaaattaaaacctgcctttctgccaggatcacatcactggaagctccatgactctctttttgtaaaagaaaaaaaaatcacagaaacacccacctcccaaactattctcttttacttcttcccccaagcccacccccaaatataactgttatccagaagctgttatgtcctgtttccatacatgtttttgtacttttactatatctacatacatcaattaaacttatgtcctattgttttgtgaatttatatttgcgtatacattatcatatgtaaaatttgcatttttttattgaaaattatgtttcttgagatttatccacattgaaacatggagctctaaatcgttaattttaaccgctatagagtattccataatttgaataaagcataatttgtttgtacaatctcccgccaagggaaaattatttccacactcatcatgacaaggagcactgcaaaaataaaaataaaaattacattcatacatgttt

SEQ ID NO: 13 - DNA, 6567 bp, Homo Sapiens
ataatgatgttgaaaatgatcttcaatcagacatcagttgagcactgcaagacacacacggagaagcagaaagaatgcaacttcttcttaatagctgttcgcctggcttcactgaaccagatcttggatccttgggtttacctgctgttaagaaagatccttcttcgaaagttttgccaggtagcaaatgctgtctccagctgctctaatgatggacagaaagggcagcctatctcattatctaatgaaataatacagacagaagcatgaaagaaaacacttaacttgcatgtgcacagcttttggtaacaaatatcgctaaaccttactgtgaatttaggcatctctggcatgccactgtttatgcattgaagtggaatttttggtataaagctaaatggtcttagaagcatagaaaatccctatgtgccaaaagtagtgaaacacaaacaaaggaaaatatattaataacagtctagtgtttttgttgagtctgccattcgtagctgaatatgtgattaattatgtgatgaaaacattttttataaatgatcttggtctattggggagcggggatagttaatattccagtacactgaatacatgaggaatttaaccacatacatcattgaagacaagggatagcagtttgtttttattcaaagacattgctgtgttctctttcattgcctctctcgctttctgtcacttttttcctccttacattaaagaaaagtttaattacagttaaaaatgtataatgtatttataatattcatcgataccattattcaaatattgctcaatacagcaaattagctcctaacctaacaaagtttaagtttacttggattgataattaggtttactctttatctgaataagaaccaattccatttgtttgaaatatggagtttgtgactacccaaattgctaattattctttcttttgaatatattttacatttctatgagcctaaggaagattcatgaaactgacctatgagagtcgtgaagtggtttttcagaatgctatgtaaggaccgatttgagcactaactataggtactctgaatatatatttcccttgattattcaccaaaagtgttccccagtctttgactctttaaattccaatactgattccaaaacaaataaatattttgaagactcaatgaatactttccatattttggcctatttatataagaaagttaataacattgacccttcacagctcttctgcctgctcctcaaagtggctctatctaaatatttattactaaaatgtttttcctacagtctacatgaatacaaacctcaatagctaagcttgacgtatttgtgcacaagtagatcactacattaagttttgggaattgcacttcttaaaaatgtctccccaccaaacatagtaatcctgtagttatgcctacacaaagcttgccatattctttggtgcattcattttgtaaacccattaactttttattgtgaagattttcatttgcagtttcttgcactgcttttctagttttttaaaagcttgagatttatttatacttcttgtagtaactgcatatttctgtgtgtgtttagtggtaaagaattaattttgataggtacaatatgtctatcagattgatatatacaccagcctatgtcaattggggctaattattttaaatgaccatgtcaaattgaatttggagacaaaatctgttgagagtgcttatgtaattaatgatggttctactaatctaaattttggaaaaggtgataaatagactatactaaaatctctctatgccatagaattggattatcctgtaggtcatctcattgggtataagacaaaactacctactttttttcaaaagtgcactgaaatcacataataaagaggctttacctcttggttggtcctgtgaccctaagttctagtcagatagacacagaggcaatgtgaatttgagtggcatgagcatgattaggttattccttccagcatctagtatagcacctggaatatagaaactgtctaatacatatttattcagtgaatcaatgagcagaagtttgccaggacagtacacattggcaaggcacataccatatgattgaagtgcttcatgccattacagtccatcaggctgataaagtgaattatttctgattatttaattacagaaatatgaatttatcttcaagggtgtagtgtcatactgctgtacaacacagtgctttatttatactaataatttaggagactgatacttccaaatgatagtggacattactatcacaagaatatcacttttcatcaaactgcaaaaatacagaaaggcaaaaaacctgacacttattcttaactgcaaattaaattcctgcccaggggatatattttaggaggggatgaatggcagcttttgtgttttttttaacaagcttgaaagggaggtggaaaacaaagaaattatgtaaatggcatatgagttttattatctaggcattcgttagtatggggaaacctgataagaaatgaaaatcccaaatgatttcagccttttcatgatggttgaggttagatttcagagatgtacagagactagagcggtggttagaaagaggatatatgtagtcacagcagaaagacgtgtctaagtttaattttattggctttcaagttcactcatgtatacttagtttgtccatacatatgtctaatcaggaaaaatgcatgtatagattatgacaattcctgaattttgaagtattggttaaaagacaattaaaggccaagaaaaccatggtggaagaagtaagcgaatgaaatgtagaaatatatgtaaaattagcaagtgtcaattttaccaagtagtgttgattttccaaacaatgaatttatatactatgctgagtcacagagaagaatgatcacatgttacttaatgagagcagtttacttttcaaataaaataggtatgatgaatgtcttaaaaatatcttgaagttgaagaaacaaaaatgagttatctcaatatttaccaagttaacctagtgctgtatatatcccaagatattttaggtaaatgtaagtgtttaatcatgccagatttaaactagtctgaaatatagggtatacatatatttctacttacatttctttattttatgaaatatccgaccatgttgcagaaaataatgcaaaacctcatgtaagttaactatgaaagatcctgtgagcacattggcattgagtgacagacaaactaaaaactggcaaacagtattttaataagggggtcactctgtggcagtattctaatattggattttcaagtagattaggctttttatttattcaacgctttttataattttgttctttttgactccaaattattggtcagctttcaaccttctccacatcagcaatcactaatagttcttttggttgagatcaactcagaaaaagaaaatagaaaacttttgttctttgaaattttagacatgcataatatctatttattttcataatttaaccccaaaagcttctcctgcaatacacaggattctaggagctgaatgacacagggagactacagagtatttattattacaaacacataaaaagcctaacttgaagaattaaaatttctattttttatctgtataacaagtacaaaccatcaacaatgacaaattttcacagctgcttgtttattgcttgttttatatgtttacatatctcaaaatctgttaaaactgaggtctaaaaaatgtgcagaattgtgcaactgtggcctagtccataagacttttctgagttgcaacaaagtgctgacaaagtgacagatgtctgtgatgtttcatgacataggttatgatcgagccaatttacaaaatttaataaccaacctaaactcacatacatatttgttaagaagctgacatcagttcagtattgtaaggaaactaactaggtggtgatgatgataaaagaattagggaaaatattttatttgatatattcccttttataattcctaaaatgaagattctatttaagggttataatttatataagtttagtcatataccattacattatgataccataagcagagtgcattatgattctctagaaatataattcaatcagatatgtattatatttatttatgtcacacattttctttactgagaataaaaattatcttattttcagaagctttgtatcaatcagtttcatgtaataagcaacccagatatctactagattatgtatttcttcatttgaaactacactgttttctcctagtccccttgactctactgtgcttatccattctttcacagaaagaaagtaacagacataattcctgttgatgaggctgggattgtttttaagaggagagataataacttcatatttttaaagtgccagtagcctaatatgtgaaacagatcagaatctgttgtgtagtaagtctgctttgttgaagaatttattatgggagtaaagataagaaggaaagagatcaccatcagaaacaagtcagccttttcatgcttttttgagcatttttggagatgattccacttctcaagttattatcatttgtgcatctcttcaatgctattgttaaatgctttagaattagaatattttgatcctttaattaaagtaagccaaacgtctaggcaaaaacagccaatcattaaactttaatagtaattcaaatatagatttctcatacagttttccatgtctgtagaaatcaaagttgtaatgttaagcagagggaaatgcgtgtgatttactaatacacttcaacgttctacttttgaaaggatactcatgtgggtggggcagagaacatagaaaaagatatgatggaaaacctgtccattttctacctgttaaccttcatcattttgtgccaggccctggaagcaaagagaggaagggaccgactgcatttatctttgaacacttgagcatcagtagtactactgagtggccaggggtcttgtctgtcaaagcaaatgataagttcactcaggccattattgactgctgaactctcttccttcccaactcttccttgaaagagaaaaaaatactttgccttcttgctctccttatcaaatgtttttgtacaaatagtgtaagcctgtttaagcaaaccaattaaaataggcactgattattttgatctgtttgtaacaaatgaatgtaagtactatttacatggtgtgcctaggaggagctgaaatcattggcactttaatccatattgtaaagatcagtatcaaaagcatagtgttcttcacctctcctcctcagcatccatctctatatacttgattaaatggaaaagtctcttttatcacctctatgtaaagttttatgggtagttatcgtcagtgtatttaaatatatcttctagtatgttttaaaggctggtcttcaatactgtggagacaaaaaataaaagagcgtatgaaaagtacgttagacttttgctggcattcaagtcatggctagtctgtgtatttaataaatgtgtgttatttatgtcgtgtttgtcaatggaaaataaagttgaatattctgaaatgtcgctgtgttttcttcctgatgtcaactcacgtcaggaatactttacctataactatgttaagtattttgctgaaatcccatttgatgtgctttgtcaaataatagcacaatgtaatgcaacagccccatgctatctggaaaaattagatcattttgttttatattccataatcatgtgcataacaaattattgttgatttaatataaatatgagagttgctcttttccgattttcaacatgatgtttctttgttttattatctcaagattattgttcttaacacagagtgcatttataaattattgtaagcttagttatttccctttcttgtggattttggtgtaatttacaggcagccaaaataagaattgtccttgctcatagtacaaccctttaattgacttgtgcctgagttttggtgtgagctgagtattggtggttatgaatactgggatccccagctatttgttcatgtacttattgaggtcactattggtcttccttcttcaatgaagggagaagctctgtcaaatgtagactgcaaagtcaagctcattaaaagtatccctctttcttcgtgagaaaagatatccaaggagaattctgtccctggttcctcccatgtgggggttctaggcctaggaccagatcttattgtctctacaatcttgattctttagtatttcaggataggcagagttttgttaaagtatacaaaaatcatagctagataagagagataacagaaataagttccagtgttctatactcctgtaggatgactacagttaatgctaatatacagttacaaatagctagaaggaggctattgaatgttcccaacacaaagaaatgataaatgtttgagatgatgatgtgctaattaccctcatctgaccagtatacattatatgtatcgaaacatcgctctgtgccc

SEQ ID NO: 14 - DNA, 147 bp, Homo Sapiens
gatactcatgtgggtggggcagagaacatagaaaaagatatgatggaaaacctgtccattttctacctgttaaccttcatcattttgtgccaggccctggaagcaaagagaggaagggaccgactgcatttatctttgaacacttga

SEQ ID NO: 15 - DNA, 225 bp, Homo Sapiens
aagtgttccccagtctttgactctttaaattccaatactgattccaaaacaaataaatattttgaagactcaatgaatactttccatattttggcctatttatataagaaagttaataacattgacccttcacagctcttctgcctgctcctcaaagtggctctatctaaatatttattactaaaatgtttttcctacagtctacatgaatacaaacctcaatag

SEQ ID NO: 16 - DNA, 470 bp, Homo Sapiens
ttctggaggccggcaagttcaggatcagggtgccagcagattcggtgtctggaggaattttggggaaattaaaacctgcctttctgccaggatcacatcactggaagctccatgactctctttttgtaaaagaaaaaaaatcacagaaacacccacctcccaaactattctcttttacttcttcccccaagcccacccccaaatataactgttatccagaagctgttatgtcctgtttccatacatgtttttgtacttttactatatctacatacatcaattaaacttatgtcctattgttttgtgaatttatatttgcgtatacattatcatatgtaaaatttgcatttttttattgaaaattatgtttcttgagatttatccacattgaaacatggagctctaaatcgttaattttaaccgctatagagtattccataatttgaataaagcataatttgtttgtacaa

SEQ ID NO: 17 - DNA, 478 bp, Homo Sapiens
gggcctgatggtatgtgtttttgtcatgcatggaggcaggtccccaggacttggtgcagtcctcatgatagaggaattttagggaaattaaaacctgcctttctgccaggatcacatcactggaagctccatgactctctttttgtaaaagaaaaaaaaaacacagaaacacccacctcccaaactattctcttttacttcttcccccaagcccacccccaaatataactgttatccagaagctgttatgtcctgtttccatacatgtttttgtactcttactatatctacatacatcaattaaacttatgtcctattgttttgtgaatttatatttgcgtatacattatcatatgtaaaatttgcatttttttattgaaaattatgtttcttgagatttatccacattgaaacatggagctctaaatcgttaattttaaccgctatagagtattccataattcgaataaagcataat

SEQ ID NO: 18 - DNA, 502 bp, Homo Sapiens
atgatgagaactagaagactcaaagagcaattctggaggccggcaagttcaggatcagggtgccagcagattcggtgtctggaggaattttggggaaattaaaacctgcctttctgccaggatcacatcactggaagctccatgactctctttttgtaaaagaaaaaaaatcacagaaacacccacctcccaaactattctcttttacttcttcccccaagcccacccccaaatataactgttatccagaagctgttatgtcctgtttccatacatgtttttgtacttttactatatctacatacatcaattaaacttatgtcctattgttttgtgaatttatatttgcgtatacattatcatatgtaaaatttgcatttttttattgaaaattatgtttcttgagatttatccacattgaaacatggagctctaaatcgttaattttaaccgctatagagtattccataatttgaataaagcataatttgtttgtacaaaa

SEQ ID NO: 19 - DNA, 777 bp, Homo Sapiens
cctaggagatgggcctgatggtatgtgtttttgtcatgcatggaggcaggtccccaggacttggtgcagtcctcatgatagaggaattttagggaaattaaaacctgcctttctgccaggatcacatcactggaagctccatgactctctttttgtaaaagaaaaaaaaaacacagaaacacccacctcccaaactattctcttttacttcttcccccaagcccacccccaaatataactgttatccagaagctgttatgtcctgtttccatacatgtttttgtactcttactatatctacatacatcaattaaacttatgtcctattgttttgtgaatttatatttgcgtatacattatcatatgtaaaatttgcatttttttattgaaaattatgtttcttgagatttatccacattgaaacatggagctctaaatcgttaattttaaccgctatagagtattccataattcgaataaagcataatcttgttcgtgcagccaaacagcgattaaccggggcgaatccgaccttacatttcccctgaaatcccacaaaaaatgctttttgctacacccgccataaaatgtcagcgctggcccatcaccacttcatcgagtgttagtgatcaaaatggcgaacgaccacggcgcacttttcgtgggggccatataaccatttctccatgaacaaacgaagccatgcagtcgcatgcagcacatacaacgtacacggtctcaattgtgtacatcatcgttacaacaatcggcacccgg

SEQ ID NO: 20 - DNA, 1638 bp, Homo Sapiens
gcgtctgccccctcccgctgcggctctctggacgccatcccctcctcacctcgaagccaacatgaaggagacccggggctacggaggggatgcccccttctgcacccgcctcaaccactcctacacaggcatgtgggcgcccgagcgttccgccgaggcgcggggcaacctcacgcgccctccagggtctggcgaggattgcggatcggtgtccgtggccttcccgatcaccatgctgctcactggtttcgtgggcaacgcactggccatgctgctcgtgtcgcgcagctaccggcgccgggagagcaagcgcaagaagtccttcctgctgtgcatcggctggctggcgctcaccgacctggtcgggcagcttctcaccaccccggtcgtcatcgtcgtgtacctgtccaagcagcgttgggagcacatcgacccgtcggggcggctctgcacctttttcgggctgaccatgactgttttcgggctctcctcgttgttcatcgccagcgccatggccgtcgagcgggcgctggccatcagggcgccgcactggtatgcgagccacatgaagacgcgtgccacccgcgctgtgctgctcggcgtgtggctggccgtgctcgccttcgccctgctgccggtgctgggcgtgggccagtacaccgtccagtggcccgggacgtggtgcttcatcagcaccgggcgagggggcaacgggactagctcttcgcataactggggcaaccttttcttcgcctctgcctttgccttcctggggctcttggcgctgacagtcaccttttcctgcaacctggccaccattaaggccctggtgtcccgctgccgggccaaggccacggcatctcagtccagtgcccagtggggccgcatcacgaccgagacggccattcagcttatggggatcatgtgcgtgctgtcggtctgctggtctccgctcctgataatgatgttgaaaatgatcttcaatcagacatcagttgagcactgcaagacacacacggagaagcagaaagaatgcaacttcttcttaatagctgttcgcctggcttcactgaaccagatcttggatccttgggtttacctgctgttaagaaagatccttcttcgaaagttttgccagttctggaggccggcaagttcaggatcagggtgccagcagattcggtgtctggaggaattttggggaaattaaaacctgcctttctgccaggatcacatcactggaagctccatgactctctttttgtaaaagaaaaaaaaatcacagaaacacccacctcccaaactattctcttttacttcttcccccaagcccacccccaaatataactgttatccagaagctgttatgtcctgtttccatacatgtttttgtacttttactatatctacatacatcaattaaacttatgtcctattgttttgtgaatttatatttgcgtatacattatcatatgtaaaatttgcatttttttattgaaaattatgtttcttgagatttatccacattgaaacatggagctctaaatcgttaattttaaccgctatagagtattccataatttgaataaagcataatttgtttgtacaatctcccgccaagggaaaattatttccaca

SEQ ID NO: 21 - DNA, 1663 bp, Homo Sapiens
gcgtctgccccctcccgctgcggctctctggacgccatcccctcctcacctcgaagccaacatgaaggagacccggggctacggaggggatgcccccttctgcacccgcctcaaccactcctacacaggcatgtgggcgcccgagcgttccgccgaggcgcggggcaacctcacgcgccctccagggtctggcgaggattgcggatcggtgtccgtggccttcccgatcaccatgctgctcactggtttcgtgggcaacgcactggccatgctgctcgtgtcgcgcagctaccggcgccgggagagcaagcgcaagaagtccttcctgctgtgcatcggctggctggcgctcaccgacctggtcgggcagcttctcaccaccccggtcgtcatcgtcgtgtacctgtccaagcagcgttgggagcacatcgacccgtcggggcggctctgcacctttttcgggctgaccatgactgttttcgggctctcctcgttgttcatcgccagcgccatggccgtcgagcgggcgctggccatcagggcgccgcactggtatgcgagccacatgaagacgcgtgccacccgcgctgtgctgctcggcgtgtggctggccgtgctcgccttcgccctgctgccggtgctgggcgtgggccagtacaccgtccagtggcccgggacgtggtgcttcatcagcaccgggcgagggggcaacgggactagctcttcgcataactggggcaaccttttcttcgcctctgcctttgccttcctggggctcttggcgctgacagtcaccttttcctgcaacctggccaccattaaggccctggtgtcccgctgccgggccaaggccacggcatctcagtccagtgcccagtggggccgcatcacgaccgagacggccattcagcttatggggatcatgtgcgtgctgtcggtctgctggtctccgctcctgataatgatgttgaaaatgatcttcaatcagacatcagttgagcactgcaagacacacacggagaagcagaaagaatgcaacttcttcttaatagctgttcgcctggcttcactgaaccagatcttggatccttgggtttacctgctgttaagaaagatccttcttcgaaagttttgccaggagatggggcctgatggaaggtgtttttgtcatgcatggaggcaggtccccaggacttggtgcagttctcatgatagaggaattttggggaaattaaaacctgcctttctgccaggatcacatcactggaagctccatgactctctttttgtaaaagaaaaaaaaatcacagaaacacccacctcccaaactattctcttttacttcttcccccaagcccacccccaaatataactgttatccagaagctgttatgtcctgtttccatacatgtttttgtacttttactatatctacatacatcaattaaacttatgtcctattgttttgtgaatttatatttgcgtatacattatcatatgtaaaatttgcatttttttattgaaaattatgtttcttgagatttatccacattgaaacatggagctctaaatcgttaattttaaccgctatagagtattccataatttgaataaagcataatttgtttgtacaatctcccgccaagggaaaattatttccaca

SEQ ID NO: 22 - protein, 382 aa, Homo Sapiens
MKETRGYGGDAPFCTRLNHSYTGMWAPERSAEARGNLTRPPGSGEDCGSVSVAFPITMLLTGFVGNALAMLLVSRSYRRRESKRKKSFLLCIGWLALTDLVGQLLTTPVVIVVYLSKQRWEHIDPSGRLCTFFGLTMTVFGLSSLFIASAMAVERALAIRAPHWYASHMKTRATRAVLLGVWLAVLAFALLPVLGVGQYTVQWPGTWCFISTGRGGNGTSSSHNWGNLFFASAFAFLGLLALTVTFSCNLATIKALVSRCRAKATASQSSAQWGRITTETAIQLMGIMCVLSVCWSPLLIMMLKMIFNQTSVEHCKTHTEKQKECNFFLIAVRLASLNQILDPWVYLLLRKILLRKFCQFWRPASSGSGCQQIRCLEEFWGN

SEQ ID NO: 23 - protein, 436 aa, Homo Sapiens
MKETRGYGGDAPFCTRLNHSYTGMWAPERSAEARGNLTRPPGSGEDCGSVSVAFPITMLLTGFVGNALAMLLVSRSYRRRESKRKKSFLLCIGWLALTDLVGQLLTTPVVIVVYLSKQRWEHIDPSGRLCTFFGLTMTVFGLSSLFIASAMAVERALAIRAPHWYASHMKTRATRAVLLGVWLAVLAFALLPVLGVGQYTVQWPGTWCFISTGRGGNGTSSSHNWGNLFFASAFAFLGLLALTVTFSCNLATIKALVSRCRAKATASQSSAQWGRITTETAIQLMGIMCVLSVCWSPLLIMMLKMIFNQTSVEHCKTHTEKQKECNFFLIAVRLASLNQILDPWVYLLLRKILLRKFCQEMGPDGRCFCHAWRQVPRTWCSSHDRGILGKLKPAFLPGSHHWKLHDSLFVKEKKITETPTSQTILFYFFPQAHPQI

SEQ ID NO: 24 - DNA, 20 bp, Artificial
  primer
tcgaaagttttgccagatga

SEQ ID NO: 25 - DNA, 23 bp, Artificial
  primer
tgaaggatcaatcacagtagagg

SEQ ID NO: 26 - DNA, 26 bp, Artificial
  probe
cagagagcaagctcctcttcttccca

SEQ ID NO: 27 - DNA, 20 bp, Artificial
  primer
tcgaaagttttgccagatga

SEQ ID NO: 28 - DNA, 20 bp, Artificial
  primer
ccatgcatgacaaaaacacc

SEQ ID NO: 29 - DNA, 25 bp, Artificial
  probe
agagagcaagagatggggcctgatg

SEQ ID NO: 30 - DNA, 20 bp, Artificial
  primer
tccttgggtttacctgctgt

SEQ ID NO: 31 - DNA, 21 bp, Artificial
  primer
tgacaaaaacaccttccatca

SEQ ID NO: 32 - DNA, 25 bp, Artificial
  probe
ttcgaaagttttgccaggagatggg

SEQ ID NO: 33 - DNA, 20 bp, Artificial
  primer
agcgaccatttggaaagatg

SEQ ID NO: 34 - DNA, 20 bp, Artificial
  primer
tggcagaaaggcaggtttta

SEQ ID NO: 35 - DNA, 26 bp, Artificial
  probe
tcagagagcaagaggaattttgggga

SEQ ID NO: 36 - DNA, 20 bp, Artificial
  primer
tccttgggtttacctgctgt

SEQ ID NO: 37 - DNA, 21 bp, Artificial
  primer
ccatcattagagcagctggag

SEQ ID NO: 38 - DNA, 26 bp, Artificial
  probe
cgaaagttttgccaggtagcaaatgc

SEQ ID NO: 39 - DNA, 21 bp, Artificial
  primer
gaaagttttgccagatgagaa

SEQ ID NO: 40 - DNA, 21 bp, Artificial
  primer
ggcagaaaggcaggttttaat

SEQ ID NO: 41 - DNA, 26 bp, Artificial
  probe
tcagagagcaagaggaattttgggga

SEQ ID NO: 42 - DNA, 20 bp, Artificial
  primer
tccttgggtttacctgctgt

SEQ ID NO: 43 - DNA, 21 bp, Artificial
  primer
ggcagaaaggcaggttttaat

SEQ ID NO: 44 - DNA, 25 bp, Artificial
  probe
agttttgccaggaggaattttgggg

SEQ ID NO: 45 - DNA, 20 bp, Artificial
  primer
ccttgggtttacctgctgtt

SEQ ID NO: 46 - DNA, 22 bp, Artificial
  primer
ctgcagattaattgctctctga

SEQ ID NO: 47 - DNA, 27 bp, Artificial
  probe
tccttcttcgaaagttttgccagatga

SEQ ID NO: 48 - DNA, 20 bp, Artificial
  primer
gttcctcaaccttgatgtgg

SEQ ID NO: 49 - DNA, 20 bp, Artificial
  primer
attcagggaagcaggaattg

SEQ ID NO: 50 - DNA, 29 bp, Artificial
  probe
ccatttggaaagataatgaaagaacggag

SEQ ID NO: 51 - DNA, 20 bp, Artificial
  primer
tcgaaagttttgccagatga

SEQ ID NO: 52 - DNA, 20 bp, Artificial
  primer
caccctgatcctgaacttgc

SEQ ID NO: 53 - DNA, 25 bp, Artificial
  probe
actcagagagcaattctggaggccg

SEQ ID NO: 54 - DNA, 19 bp, Artificial
  primer
gatcagggtgccagcagat

SEQ ID NO: 55 - DNA, 20 bp, Artificial
  primer
atgtgatcctggcagaaagg

SEQ ID NO: 56 - DNA, 25 bp, Artificial
  probe
cggtgtctggaggaattttggggaa

SEQ ID NO: 57 - DNA, 18 bp, Artificial
  primer
aggtccccaggacttggt

SEQ ID NO: 58 - DNA, 20 bp, Artificial
  primer
atgtgatcctggcagaaagg

SEQ ID NO: 59 - DNA, 28 bp, Artificial
  probe
cagttctcatgatagaggaattttgggg

SEQ ID NO: 60 - DNA, 24 bp, Artificial
  primer
tttacctgctgttaagaaagatcc

SEQ ID NO: 61 - DNA, 27 bp, Artificial
  primer
catcatatctttttctatgttctctgc

SEQ ID NO: 62 - DNA, 24 bp, Artificial
  probe
ttgccaggatactcatgtgggtgg

SEQ ID NO: 63 - DNA, 22 bp, Artificial
  primer
tgggtttacctgctgttaagaa

SEQ ID NO: 64 - DNA, 27 bp, Artificial
  primer
tcagtattggaatttaaagagtcaaag

SEQ ID NO: 65 - DNA, 26 bp, Artificial
  probe
cgaaagttttgccagaagtgttcccc